(19) United States
(12) Patent Application Publication
(10) Pub. No.: US 2012/0088664 A1
(43) Pub. Date: Apr. 12, 2012

(54) Antifungal 1,2,4-triazolyl derivatives having a 5-sulfur subtituent

(75) Inventors: Jens Renner, Dirmstein (DE); Jochen Dietz, Karlsruhe (DE); Alice Glaettli, Frankfurt (DE); Thomas Grote, Wachenheim (DE); Wassilios Grammenos, Ludwigshafen (DE); Bernd Mueller, Frankenthal (DE); Jan Klaas Lohmann, Mannheim (DE); Sarah Ulmschneider, Dirmstein (DE); Marianna Vrettou-Schultes, Mannheim (DE)

(73) Assignee: BASF SE, Ludwigshafen (DE)

(21) Appl. No.: 13/378,165

(22) PCT Filed: Jun. 17, 2010
(86) PCT No.: PCT/EP2010/058538

§ 371 Date: Dec. 14, 2011

(30) Foreign Application Priority Data
Jun. 18, 2009 (EP) 09163168.9

(51) Int. Cl. C07D 249/12; A01N 43/653; A01N 25/26; A61P 35/00; A61P 31/12; A61P 31/10; A61K 31/4196; A01P 3/00

(52) U.S. Cl. 504/100; 548/263.2; 514/384

(57) ABSTRACT

The present invention relates to novel triazole compounds of the formulae I and II as defined below which carry a sulfur substituent, to agricultural compositions containing them, to their use as fungicides and to intermediate compounds used in the method of producing them.

The present invention relates to novel triazole compounds of the formulae I and II as defined below which carry a sulfur substituent, to agricultural compositions containing them, to their use as fungicides and to intermediate compounds used in the method of producing them.

The control of plant diseases caused by phythopathogenic fungi is extremely important for achieving high crop efficiency. Plant disease damage to ornamental, vegetable, field, cereal, and fruit crops can cause significant reduction in productivity and thereby result in increased costs to the consumer.

WO96/16048, WO97/41107, WO97/42178, WO97/43269, WO97/44331, WO97/44332 and WO99/05149 describe sulfurized triazolyl derivatives. The compounds are used for combating harmful fungi.

There is a continuous need for new compounds which are more effective, less costly, less toxic, environmentally safer and/or have different modes of action.

Accordingly, it is an object of the present invention to provide compounds having a better fungicidal activity and/or a better crop plant compatibility.

Surprisingly, these objects are achieved by triazole compounds of the general formulae I and II, defined below, and by the agriculturally acceptable salts of the compounds I and II.

Accordingly, the present invention relates to triazole compounds of the formulae I and II and to agriculturally useful salts thereof

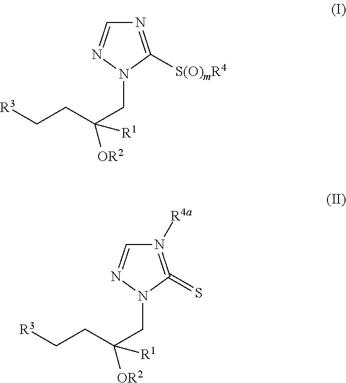

wherein
R¹ is selected from C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₃-C₁₀-cycloalkyl-C₁-C₄-alkyl, C₃-C₁₀-halocycloalkyl-C₁-C₄-alkyl, where the cycloalkyl moiety in the 4 last-mentioned radicals may carry 1, 2, 3 or 4 substituents R⁶, phenyl which may carry 1, 2, 3, 4 or 5 substituents R⁵, and a saturated, partially unsaturated or maximum unsaturated 3-, 4-, 5-, 6- or 7-membered heterocyclic ring containing 1, 2 or 3 heteroatoms or heteroatom-containing groups selected from N, O, S, SO and SO₂ as ring members, where the heterocyclic ring may carry 1, 2 or 3 substituents R⁵;
R² is selected from hydrogen and a protective group;
R³ is selected from C₃-C₁₀-cycloalkyl, C₃-C₁₀-halocycloalkyl, where the cycloalkyl moiety in the 2 last-mentioned radicals may carry 1, 2, 3 or 4 substituents R⁶, C₃-C₁₀-cycloalkenyl, C₃-C₁₀-halocycloalkenyl, where the cycloalkenyl moiety in the 2 last-mentioned radicals may carry 1, 2, 3 or 4 substituents R⁶, aryl which may carry 1, 2 or 3 substituents R⁷, and a saturated, partially unsaturated or maximum unsaturated 3-, 4-, 5-, 6- or 7-membered heterocyclic ring containing 1, 2 or 3 heteroatoms or heteroatom-containing groups selected from N, O, S, SO and SO₂ as ring members, where the heterocyclic ring may carry 1, 2 or 3 substituents R⁸;
R⁴ is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R⁹, and a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R⁹; or, in case m is 0, may also be selected from —C(═O)R¹⁰, —C(═S)R¹⁰, —S(O)₂R¹⁰, —CN, —P(═O)R¹¹R¹², M and a group of the formula III

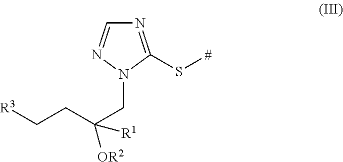

wherein
R¹, R² and R³ are as defined for formulae I and II; and
# is the attachment point to the remainder of the molecule;
R^{4a} is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R⁹, a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R⁹, —C(═O)R¹⁰, —C(═S)R¹⁰, —S(O)₂R¹⁰, —CN, —P(=Q)R¹¹R¹² and M;
each R⁵ is independently selected from halogen, nitro, CN, C₁-C₄-alkyl, C₁-C₄-haloalkyl, C₁-C₄-alkoxy, C₁-C₄-haloalkoxy and NR¹³R¹⁴;
each R⁶ is independently selected from nitro, CN, C₁-C₄-alkyl, C₁-C₄-haloalkyl, C₁-C₄-alkoxy, C₁-C₄-haloalkoxy and NR¹³R¹⁴;
each R⁷ is independently selected from halogen, nitro, CN, C₁-C₄-alkyl, C₁-C₄-haloalkyl, C₂-C₄-alkenyl, C₂-C₄-haloalkenyl, C₁-C₄-alkoxy, C₁-C₄-haloalkoxy, phenyl, phenoxy, where the phenyl moiety in the two last-mentioned phenyl radicals may carry 1, 2 or 3 substituents R⁵, and NR¹³R¹⁴;
each R⁸ is independently selected from halogen, nitro, CN, C₁-C₄-alkyl, C₁-C₄-haloalkyl, C₂-C₄-alkenyl, C₂-C₄-haloalkenyl, C₁-C₄-alkoxy, C₁-C₄-haloalkoxy, phenyl, phenoxy, where the phenyl moiety in the two last-mentioned phenyl radicals may carry 1, 2 or 3 substituents R⁵, and NR¹³R¹⁴;
each R⁹ is independently selected from halogen, nitro, CN, C₁-C₄-alkyl, C₁-C₄-haloalkyl, C₁-C₄-alkoxy, C₁-C₄-haloalkoxy and NR¹³R¹⁴;
R¹⁰ is selected from hydrogen, C₁-C₁₀-alkyl, C₁-C₁₀-haloalkyl, C₁-C₁₀-alkoxy, C₁-C₁₀-haloalkoxy, C₁-C₁₀-aminoalkyl, C₃-C₁₀-cycloalkyl, C₃-C₁₀-halocycloalkyl, phenyl, phenyl-C₁-C₄-alkyl, where the phenyl moiety in the 2 last-mentioned radicals may carry 1, 2, 3, 4 or 5 substituents R⁹, a 5- or 6-membered saturated, partially unsaturated or aromatic heterocyclic ring containing 1, 2 or 3 heteroatoms selected from N, O and S as ring members, where the heterocyclic ring may carry 1, 2 or 3 substituents R⁹, and NR¹³R¹⁴;
R¹¹ and R¹², independently of each other, are selected from C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₁-C₁₀-alkoxy, C₁-C₁₀-haloalkoxy, C₁-C₄-alkoxy-C₁-C₁₀-alkyl, C₁-C₄-alkoxy-C₁-C₁₀-alkoxy, C₁-C₁₀-alkylthio, C₁-C₁₀-haloalkylthio, C₂-C₁₀-alkenyloxy, C₂-C₁₀-alkenylthio, C₂-C₁₀-alkynyloxy, C₂-C₁₀-alkynylthio, C₃-C₁₀-cycloalkoxy, C₃-C₁₀-cycloalkylthio, phenyl, phenyl-C₁-C₄-alkyl, phenylthio, phenyl-C₁-C₄-alkoxy, and NR¹³R¹⁴;
each R¹³ is independently selected from hydrogen and C₁-C₈-alkyl;
each R¹⁴ is independently selected from hydrogen, C₁-C₈-alkyl, phenyl, and phenyl-C₁-C₄-alkyl;
   or R¹³ and R¹⁴ together form a linear C₄- or C₅-alkylene bridge or a group —CH₂CH₂OCH₂CH₂— or —CH₂CH₂NR¹⁵CH₂CH₂—;
each R¹⁵ is independently selected from hydrogen and C₁-C₄-alkyl;
Q is O or S;
M is a metal cation equivalent or an ammonium cation of formula (NR^{a}R^{b}R^{c}R^{d})⁺, wherein R^{a}, R^{b}, R^{c} and R^{d}, independently of each other, are selected from hydrogen, C₁-C₁₀-alkyl, phenyl and benzyl, where the phenyl moiety in the 2 last-mentioned radicals may carry 1, 2 or 3 substituents independently selected from halogen, CN, nitro, C₁-C₄-alkyl, C₁-C₄-haloalkyl, C₁-C₄-alkoxy, C₁-C₄-haloalkoxy and NR¹³R¹⁴; and
m is 0, 1, 2 or 3;
   with the proviso that R³ is not 4-chlorophenyl, 2-pyridyl or 2-furyl if R¹ is tert-butyl or optionally substituted cyclopropyl and preferably with the proviso that R⁷ is not Cl and that R³ is not a 5- or 6-membered heteroaromatic ring containing 1, 2 or 3 heteroatoms selected from O, S and N as ring members if R¹ is tert-butyl or optionally substituted cyclopropyl.

The present invention also provides the use of triazole compounds of the formulae I and II and/or their agriculturally useful salts for controlling harmful fungi.

The invention further provides fungicidal compositions comprising these triazole compounds of the formulae I and/or II (and/or also of the formula IV; see below) and/or their agriculturally acceptable salts and suitable carriers. Suitable agriculturally acceptable carriers are described below.

The compounds I and II can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds I and II can be understood as positional/double bond isomers of each other, at least in case the radicals R⁴/R^{4a} are identical. In case R⁴ (and of course also R^{4a}) is hydrogen, the respective compounds I and II are tautomers.

Suitable agriculturally useful salts are especially the salts of those cations or the acid addition salts of those acids whose cations and anions, respectively, have no adverse effect on the fungicidal action of the compounds I and II. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are primarily chloride, bromide, fluoride, hydrogen-sulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I or II with an acid of the corresponding anion, preferably hydrochloric acid, hydrobromic acid, sulfuric acid, phosphoric acid or nitric acid.

In the definitions of the variables given in the formulae above, collective terms are used which are generally representative for the substituents in question. The term Cₙ-Cₘ indicates the number of carbon atoms possible in each case in the substituent or substitutent moiety in question:

Halogen: fluorine, chlorine, bromine and iodine;

Alkyl and the alkyl moieties in alkoxy, alkoxyalkyl, alkoxyalkoxy, alkylcarbonyl, alkyl-thiocarbonyl, aminoalkyl, alkylamino, dialkylamino, alkylaminocarbonyl, dialkylamino-carbonyl, alkylthio, alkylsulfonyl and the like: saturated straight-chain or branched hydrocarbon radicals having 1 to 2 (C₁-C₂-alkyl), 2 or 3 (C₂-C₃-alkyl), 1 to 4 (C₁-C₄-alkyl), 1 to 6 (C₁-C₆-alkyl), 1 to 8 (C₁-C₈-alkyl) or 1 to 10 (C₁-C₁₀-alkyl) carbon atoms. C₂-C₃-Alkyl is ethyl, n-propyl or isopropyl. C₁-C₂-Alkyl is methyl or ethyl. C₁-C₄-Alkyl is methyl, ethyl, propyl, isopropyl, butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl, 2-propylheptyl, 3-propylheptyl and positional isomers thereof.

Haloalkyl: straight-chain or branched alkyl groups having 1 to 2 (C₁-C₂-haloalkyl), 1 to 3 (C₁-C₃-haloalkyl), 1 to 4 (C₁-C₄-haloalkyl), 1 to 6 (C₁-C₆-haloalkyl), 1 to 8 (C₁-C₈-haloalkyl), 1 to 10 (C₁-C₁₀-haloalkyl) or 2 to 10 (C₂-C₁₀-haloalkyl) carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoro-methyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, or pentafluoroethyl. C₁-C₃-Haloalkyl is additionally, for example, 1,1,1-trifluoroprop-2-yl, 3,3,3-trifluoropropyl or heptafluoropropyl. C₁-C₄-Haloalkyl is additionally, for example, 1-chlorobuty, 2-chlorobutyl, 3-chlorobutyl or 4-chlorobutyl.

C₁-C₁₀-Hydroxyalkyl: straight-chain or branched alkyl groups having 1 to 2 (C₁-C₂-hydroxyalkyl), 1 to 4 (C₁-C₄-hydroxyalkyl), 2 to 4 (C₂-C₄-hydroxyalkyl), 1 to 6 (C₁-C₆-hydroxyalkyl), 2 to 6 (C₂-C₆-hydroxyalkyl), 1 to 8 (C₁-C₈-hydroxyalkyl), 2 to 8 (C₂-C₈-hydroxyalkyl), 1 to 10 (C₁-C₁₀-hydroxyalkyl) or 2 to 10 (C₂-C₁₀-hydroxyalkyl) carbon atoms (as mentioned above), where at least one of the hydrogen atoms is replaced by a hydroxyl group, such as in 2-hydroxyethyl or 3-hydroxypropyl.

Alkenyl and the alkenyl moieties in alkenyloxy, alkenylthio, alkenylcarbonyl and the like: monounsaturated straight-chain or branched hydrocarbon radicals having 2 to 4 (C₂-C₄-alkenyl), 2 to 6 (C₂-C₆-alkenyl), 2 to 8 (C₂-C₈-alkenyl), 3 to 8 (C₃-C₈-alkenyl), 2 to 10 (C₂-C₁₀-alkenyl) or 3 to 10 (C₃-C₁₀-alkenyl) carbon atoms and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl, or, for example, C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;

Haloalkenyl and the haloalkenyl moieties in haloalkenyloxy, haloalkenylcarbonyl and the like: unsaturated straight-chain or branched hydrocarbon radicals having 2 to 4 (C₂-C₄-haloalkenyl), 2 to 6 (C₂-C₆-haloalkenyl), 2 to 8 (C₂-C₈-haloalkenyl) or 2 to 10 (C₂-C₁₀-haloalkenyl) carbon atoms and a double bond in any position (as mentioned above), where some or all of the hydrogen atoms in these groups may be replaced by halogen atoms as mentioned above, in particular fluorine, chlorine and bromine, for example chlorovinyl, chloroallyl and the like;

Alkynyl and the alkynyl moieties in alkynyloxy, alkynylthio, alkynylcarbonyl and the like: straight-chain or branched hydrocarbon groups having 2 to 4 (C₂-C₄-alkynyl), 2 to 6 (C₂-C₆-alkynyl), 2 to 8 (C₂-C₈-alkynyl), 3 to 8 (C₃-C₈-alkynyl), 2 to 10 (C₂-C₁₀-alkynyl) or 3 to 10 (C₃-C₁₀-alkynyl) carbon atoms and one or two triple bonds in any position, for example C₂-C₄-alkynyl, such as ethynyl, 1-propynyl, 2-propynyl, 1-butynyl, 2-butynyl, 3-butynyl, or 1-methyl-2-propynyl, or, for example, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

Haloalkynyl and the haloalkynyl moieties in haloalkynyloxy, haloalkynylcarbonyl and the like: unsaturated straight-chain or branched hydrocarbon radicals having 2 to 4 (C₂-C₄-haloalkynyl), 2 to 6 (C₂-C₆-haloalkynyl), 2 to 8 (C₂-C₈-haloalkynyl) or 2 to 10 (C₂-C₁₀-haloalkynyl) carbon atoms and one or two triple bonds in any position (as mentioned above), where some or all of the hydrogen atoms in these groups may be replaced by halogen atoms as mentioned above, in particular fluorine, chlorine and bromine;

Cycloalkyl and the cycloalkyl moieties in cycloalkoxy, cycloalkylcarbonyl and the like; monocyclic saturated hydrocarbon groups having 3 to 6 (C₃-C₆-cycloalkyl), 3 to 8 (C₃-C₈-cycloalkyl) or 3 to 10 (C₃-C₁₀-cycloalkyl) carbon ring members, such as cyclopropyl, cyclobutyl, cyclopentyl, cyclohexyl, cycloheptyl, cyclooctyl, cyclononyl and cyclodecyl;

Halocycloalkyl and the halocycloalkyl moieties in halocycloalkoxy, halocycloalkylcarbonyl and the like: monocyclic saturated hydrocarbon groups having 3 to 6 (C₃-C₆-halocycloalkyl), 3 to 8 (C₃-C₈-halocycloalkyl) or 3 to 10 (C₃-C₁₀-halocycloalkyl) carbon ring members (as mentioned above) in which some or all of the hydrogen atoms may be replaced by halogen atoms as mentioned above, in particular fluorine, chlorine and bromine;

Cycloalkenyl and the cycloalkenyl moieties in cycloalkenyloxy, cycloalkenylcarbonyl and the like; monocyclic monounsaturated hydrocarbon groups having 3 to 6 (C₃-C₆-cycloalkenyl), 3 to 8 (C₃-C₈-cycloalkenyl) or 3 to 10 (C₃-C₁₀-cycloalkenyl) carbon ring members, such as cyclopropenyl, cyclobutenyl, cyclopentenyl, cyclohexenyl, cycloheptenyl, cyclooctenyl, cyclononenyl and cyclodecenyl;

Halocycloalkenyl and the halocycloalkenyl moieties in halocycloalkenyloxy, halocycloalkenylcarbonyl and the like: monocyclic monounsaturated hydrocarbon groups having 3 to 6 (C₃-C₆-halocycloalkenyl), 3 to 8 (C₃-C₈-halocycloalkenyl) or 3 to 10 (C₃-C₁₀-halocycloalkenyl) carbon ring members (as mentioned above) in which some or all of the hydrogen atoms may be replaced by halogen atoms as mentioned above, in particular fluorine, chlorine and bromine;

C₃-C₆-cycloalkyl-C₁-C₂-alkyl: a C₁-C₂-alkyl residue, as described above, wherein one of the hydrogen atoms is replaced by a C₃-C₆-cycloalkyl group. Examples are cyclopropylmethyl, cyclobutylmethyl, cyclopentylmethyl, cyclohexylmethyl, cyclopropyl-1-ethyl, cyclobutyl-1-ethyl, cyclopentyl-1-ethyl, cyclohexyl-1-ethyl, cyclopropyl-2-ethyl, cyclobutyl-2-ethyl, cyclopentyl-2-ethyl, cyclohexyl-2-ethyl and the like. C₃-C₁₀-cycloalkyl-C₁-C₄-alkyl is a C₁-C₄-alkyl residue, as described above, wherein one of the hydrogen atoms is replaced by a C₃-C₁₀-cycloalkyl group. Examples are, apart those mentioned above for C₃-C₆-cycloalkyl-C₁-C₄-alkyl, cycloheptylmethyl, cyclooctylmethyl, cyclononylmethyl, cyclodecylmethyl, cycloheptyl-1-ethyl, cyclooctyl-1-ethyl, cyclononyl-1-ethyl, cyclodecyl-1-ethyl, cycloheptyl-2-ethyl, cyclooctyl-2-ethyl, cyclononyl-2-ethyl, cyclodecyl-2-ethyl, cyclopropyl-1-propyl, cyclopropyl-2-propyl, cyclopropyl-3-propyl, cyclobutyl-1-propyl, cyclobutyl-2-propyl, cyclobutyl-3-propyl, cyclopentyl-1-propyl, cyclopentyl-2-propyl, cyclopentyl-3-propyl, cyclohexyl-1-propyl, cyclohexyl-2-propyl, cyclohexyl-3-propyl, cycloheptyl-1-propyl, cycloheptyl-2-propyl, cycloheptyl-3-propyl, cyclooctyl-1-propyl, cyclooctyl-2-propyl, cyclooctyl-3-propyl, cyclononyl-1-propyl, cyclononyl-2-propyl, cyclononyl-3-propyl, cyclodecyl-1-propyl, cyclodecyl-2-propyl, cyclodecyl-3-propy, cyclopropyl-1-butyl, cyclopropyl-2-butyl, cyclopropyl-3-butyl, cyclopropyl-4-butyl, cyclobutyl-1-butyl, cyclobutyl-2-butyl, cyclobutyl-3-butyl, cyclobutyl-4-butyl, cyclopentyl-1-butyl, cyclopentyl-2-butyl, cyclopentyl-3-butyl, cyclopentyl-4-butyl, cyclohexyl-1-butyl, cyclohexyl-2-butyl, cyclohexyl-3-butyl, cyclohexyl-4-butyl, cycloheptyl-1-butyl, cycloheptyl-2-butyl, cycloheptyl-3-butyl, cycloheptyl-4-butyl, cyclooctyl-1-butyl, cyclooctyl-2-butyl, cyclooctyl-3-butyl, cyclooctyl-4-butyl, cyclononyl-1-butyl, cyclononyl-2-butyl, cyclononyl-3-butyl, cyclononyl-4-butyl, cyclodecyl-1-butyl, cyclodecyl-2-butyl, cyclodecyl-3-butyl, cyclodecyl-4-butyl, and the like.

C₃-C₆-halocycloalkyl-C₁-C₂-alkyl: a C₁-C₂-alkyl residue, as described above, wherein one of the hydrogen atoms is replaced by a C₃-C₆-halocycloalkyl group. Examples are 1-chlorocyclopropylmethyl, 1-chlorocyclobutylmethyl, 1-chlorocyclopentylmethyl, 1-chlorocyclohexylmethyl, 1-chlorocyclopropyl-1-ethyl, 1-chlorocyclobutyl-1-ethyl, 1-chlorocyclopentyl-1-ethyl, 1-chlorocyclohexyl-1-ethyl, 1-chlorocyclopropyl-2-ethyl, 1-chlorocyclobutyl-2-ethyl, 1-chlorocyclopentyl-2-ethyl, 1-chlorocyclohexyl-2-ethyl, 2-chlorocyclopropylmethyl, 2-chlorocyclobutylmethyl, 2-chlorocyclopentylmethyl, 2-chlorocyclohexylmethyl, 2-chlorocyclopropyl-1-ethyl, 2-chlorocyclobutyl-1-ethyl, 2-chlorocyclopentyl-1-ethyl, 2-chlorocyclohexyl-1-ethyl, 2-chlorocyclopropyl-2-ethyl, 2-chlorocyclobutyl-2-ethyl, 2-chlorocyclopentyl-2-ethyl, 2-chlorocyclohexyl-2-ethyl, 1-fluorocyclopropylmethyl, 1-fluorocyclobutylmethyl, 1-fluorocyclopentylmethyl, 1-fluorocyclohexylmethyl, 1-fluorocyclopropyl-1-ethyl, 1-fluorocyclobutyl-1-ethyl, 1-fluorocyclopentyl-1-ethyl, 1-fluorocyclohexyl-1-ethyl, 1-fluorocyclopropyl-2-ethyl, 1-fluorocyclobutyl-2-ethyl, 1-fluorocyclopentyl-2-ethyl, 1-fluorocyclohexyl-2-ethyl, 2-fluorocyclopropylmethyl, 2-fluorocyclobutylmethyl, 2-fluorocyclopentyl methyl, 2-fluorocyclohexylmethyl, 2-fluorocyclopropyl-1-ethyl, 2-fluorocyclobutyl-1-ethyl, 2-fluorocyclopentyl-1-ethyl, 2-fluorocyclohexyl-1-ethyl, 2-fluorocyclopropyl-2-ethyl, 2-fluorocyclobutyl-2-ethyl, 2-fluorocyclopentyl-2-ethyl, 2-fluorocyclohexyl-2-ethyl, and the like. C₃-C₁₀-halocycloalkyl-C₁-C₄-alkyl is a C₁-C₄-alkyl residue, as described above, wherein one of the hydrogen atoms is replaced by a C₃-C₁₀-halocycloalkyl group.

Alkoxy: an alkyl group attached via oxygen. C₁-C₂-Alkoxy is methoxy or ethoxy. C₁-C₃-Alkoxy is additionally, for example, n-propoxy or 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof. C₂-C₁₀-Alkoxy is like C₁-C₁₀-alkoxy with the exception of methoxy.

Haloalkoxy: an alkoxy radical as mentioned above which is partially or fully substituted by fluorine, chlorine, bromine and/or iodine, preferably by fluorine. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(OH201)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

Alkenyloxy: alkenyl as mentioned above which is attached via an oxygen atom, for example C₂-C₁₀-alkenyloxy, such as 1-ethenyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy and the like;

Haloalkenyloxy: an alkenyloxy radical as mentioned above which is partially or fully substituted by fluorine, chlorine, bromine and/or iodine, preferably by fluorine.

Alkynyloxy: alkynyl as mentioned above which is attached via an oxygen atom, for example C₂-C₁₀-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

Haloalkynyloxy: an alkynyloxy radical as mentioned above which is partially or fully substituted by fluorine, chlorine, bromine and/or iodine, preferably by fluorine.

Cycloalkoxy: cycloalkyl as mentioned above which is attached via an oxygen atom, for example C₃-C₁₀-cycloalkoxy or C₃-C₈-cycloalkoxy, such as cyclopropoxy, cyclopentoxy, cyclohexoxy, cycloheptoxy, cyclooctoxy, cyclononyloxy, cyclodecyloxy and the like;

Cycloalkenyloxy: cycloalkenyl as mentioned above which is attached via an oxygen atom, for example C₃-C₁₀-cycloalkenyloxy, C₃-C₈-cycloalkenyloxy or, preferably, C₅-C₆-cycloalkenyloxy, such as cyclopent-1-enoxy, cyclopent-2-enoxy, cyclohex-1-enoxy and cyclohex-2-enoxy;

Alkoxyalkyl: alkyl as defined above having 1 to 10, 1 to 8, 1 to 6 or 1 to 4, in particular 1 to 3, carbon atoms, in which one hydrogen atom is replaced by an alkoxy group having 1 to 8, 1 to 6, 1 to 4 or 1 to 3 carbon atoms, for example methoxymethyl, 2-methoxyethyl, ethoxymethyl, 3-methoxypropyl, 3-ethoxypropyl and the like.

Alkoxyalkoxy: alkoxy as defined above having 1 to 10, 1 to 8, 1 to 6 or 1 to 4, in particular 1 to 3, carbon atoms, in which one hydrogen atom is replaced by an alkoxy group having 1 to 8, 1 to 6 or in particular 1 to 4 carbon atoms, for example 2-methoxyethoxy, 2-ethoxyethoxy, 3-methoxypropoxy, 3-ethoxypropoxy and the like.

Alkylcarbonyl: group of the formula R—CO— in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples are acetyl, propionyl and the like. Examples for C₃-C₄-alkylcarbonyl are propylcarbonyl, isopropylcarbonyl, n-butylcarbonyl, sec-butylcarbonyl, isobutylcarbonyl and tert-butylcarbonyl.

Haloalkylcarbonyl: group of the formula R—CO— in which R is a haloalkyl group as defined above, for example C₁-C₁₀-haloalkyl, C₁-C₈-haloalkyl, C₁-C₆-haloalkyl, C₁-C₄-haloalkyl, C₁-C₂-haloalkyl or C₃-C₄-haloalkyl. Examples are difluoromethylcarbonyl, trifluoromethylcarbonyl, 2,2-difluoroethylcarbony, 2,2,3-trifluoroethylcarbonyl and the like.

Alkoxycarbonyl: group of the formula R—CO— in which R is an alkoxy group as defined above, for example C₁-C₁₀-alkoxy, C₁-C₈-alkoxy, C₁-C₆-alkoxy, C₁-C₄-alkoxy or C₁-C₂-alkoxy. Examples for C₁-C₄-alkoxycarbonyl are methoxycarbonyl, ethoxycarbonyl, propoxycarbonyl, isopropoxycarbonyl, n-butoxycarbonyl, sec-butoxycarbonyl, isobutoxy-carbonyl and tert-butoxycarbonyl.

Haloalkoxycarbonyl: group of the formula R—CO— in which R is a haloalkoxy group as defined above, for example C₁-C₁₀-haloalkoxy, C₁-C₈-haloalkoxy, C₁-C₆-haloalkoxy, C₁-C₄-haloalkoxy or C₁-C₂-haloalkoxy. Examples for C₁-C₄-haloalkoxycarbonyl are difluoromethoxycarbonyl, trifluoromethoxycarbonyl, 2,2-difluoroethoxycarbony, 2,2,3-trifluoroethoxycarbonyl and the like.

Alkylaminocarbonyl: group of the formula R—NH—CO— in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples for C₁-C₄-alkylaminocarbonyl are methylaminocarbonyl, ethylaminocarbonyl, propylaminocarbonyl, isopropylaminocarbonyl, butylaminocarbonyl, sec-butylaminocarbonyl, isobutylaminocarbonyl and tert-butylaminocarbonyl.

Dialkylaminocarbonyl: group of the formula RR′N—CO— in which R and R′, independently of each other, are an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples for di-(C₁-C₄-alkyl)-aminocarbonyl are dimethylaminocarbonyl, diethylaminocarbonyl, dipropylaminocarbonyl, diisopropylaminocarbonyl and dibutylaminocarbonyl.

Aminoalkyl: group of the formula R—NH₂ in which R is an alkyl group as defined above, for example C₁-C₁₀-alkyl, C₁-C₈-alkyl, C₁-C₆-alkyl, C₁-C₄-alkyl, C₁-C₂-alkyl or C₃-C₄-alkyl. Examples are aminomethyl, 1- and 2-aminoethyl, 1-, 2- and 3-aminopropyl, 1- and 2-amino1-methylethyl, 1-, 2-, 3- and 4-aminobutyl and the like.

Alkylsulfonyl: group of the formula R—S(O)₂— in which R is an alkyl group as defined above, for example C₁-C₁₀-alkyl, C₁-C₈-alkyl, C₁-C₆-alkyl, C₁-C₄-alkyl or C₁-C₂-alkyl. Examples for C₁-C₄-alkylsulfonyl are methylsulfonyl, ethylsulfonyl, propylsulfonyl, iso-propylsulfonyl, n-butylsulfonyl, sec-butylsulfonyl, isobutylsulfonyl and tert-butylsulfonyl.

Alkylthio: alkyl as defined above which is attached via a sulfur atom.

Haloalkylthio: haloalkyl as defined above which is attached via a sulfur atom.

Alkenylthio: alkenyl as defined above which is attached via a sulfur atom.

Haloalkenylthio: haloalkenyl as defined above which is attached via a sulfur atom.

Alkynylthio: alkynyl as defined above which is attached via a sulfur atom.

Haloalkynylthio: haloalkynyl as defined above which is attached via a sulfur atom.

Cycloalkylthio: cycloalkyl as defined above which is attached via a sulfur atom.

Aryl is a carbocyclic aromatic monocyclic or polycyclic ring containing 6 to 16 carbon atoms as ring members. Examples are phenyl, naphthyl, anthracenyl, phenanthrenyl, fluorenyl and azulenyl. Preferably, aryl is phenyl or naphthyl, and especially phenyl.

Phenyl-C₁-C₄-alkyl: C₁-C₄-alkyl (as defined above), where a hydrogen atom is replaced by a phenyl group, such as benzyl, phenethyl and the like.

Phenyl-C₁-C₄-alkoxy: C₁-C₄-alkoxy (as defined above), where one hydrogen atom is replaced by a phenyl group, such as benzyloxy, phenethyloxy and the like.

3-, 4-, 5-, 6- or 7-membered saturated, partially unsaturated or maximum unsaturated carbocyclic radical: cyclopropyl, cyclobutyl, cyclopentyl, cyclohexyl, cycloheptyl, cyclo-propenyl, cyclobutenyl, cyclopentenyl, cyclohexenyl, cycloheptenyl, cyclobutadienyl, cyclopentadienyl, cyclohexadienyl, cycloheptadienyl or cycloheptatrienyl. Formally, phenyl is also included in this definition, but as it is also encompassed in the term aryl, it is not listed here.

3-, 4-, 5-, 6- or 7-membered saturated, partially unsaturated or maximum unsaturated heterocycle which contains 1, 2 or 3 heteroatoms or heteroatom containing groups selected from oxygen, nitrogen (as N or NR) and sulfur (as S, SO or SO₂) and optionally 1 or 2 groups selected from C(═O) and C(═S) as ring members:
three- or four-membered saturated or partially unsaturated heterocycle (hereinbelow also referred to as heterocyclyl) which contains 1, 2 or 3 heteroatoms from the group consisting of oxygen, nitrogen (as N or NR) and sulfur (as S, SO or SO₂) and optionally 1 or 2 groups selected from C(═O) and C(═S) as ring members: for example monocyclic saturated or partially unsaturated heterocycles which, in addition to carbon ring members, contain one to three nitrogen atoms and/or one oxygen or sulfur atom or one or two oxygen and/or sulfur atoms and optionally 1 or 2 groups selected from C(═O) and C(═S), for example 2-oxiranyl, 2-thiiranyl, 1- or 2-aziridinyl, 1-, 2- or 3-azetidinyl, five- or six-membered saturated or partially unsaturated heterocycle (hereinbelow also referred to as heterocyclyl) which contains 1, 2 or 3 heteroatoms from the group consisting of oxygen, nitrogen (as N or NR) and sulfur (as S, SO or SO₂) and optionally 1 or 2 groups selected from C(═O) and C(═S) as ring members: for example monocyclic saturated or partially unsaturated heterocycles which, in addition to carbon ring members, contain one to three nitrogen atoms and/or one oxygen or sulfur atom or one or two oxygen and/or sulfur atoms and optionally 1 or 2 groups selected from C(═O) and C(═S), for example 2-tetrahydrofuranyl, 3-tetrahydrofuranyl, 3-tetrahydrofuran-2-onyl, 4-tetrahydrofuran-2-onyl, 5-tetrahydrofuran-2-onyl, 2-tetrahydrofuran-3-onyl, 4-tetrahydrofuran-3-onyl, 5-tetrahydrofuran-3-onyl, 2-tetrahydrothienyl, 3-tetrahydrothienyl, 3-tetrahydrothien-2-onyl, 4-tetrahydrothien-2-onyl, 5-tetrahydrothien-2-onyl, 2-tetrahydrothien-3-onyl, 4-tetrahydrothien-3-onyl, 5-tetrahydrothien-3-onyl, 2-pyrrolidinyl, 3-pyrrolidinyl, 1-pyrrolidin-2-onyl, 3-pyrrolidin-2-onyl, 4-pyrrolidin-2-onyl, 5-pyrrolidin-2-onyl, 1-pyrrolidin-3-onyl, 2-pyrrolidin-3-onyl, 4-pyrrolidin-3-onyl, 5-pyrrolidin-3-onyl, 1-pyrrolidin-2,5-dionyl, 3-pyrrolidin-2,5-dionyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals;
a seven-membered saturated or partially unsaturated heterocycle which contains 1, 2 or 3 heteroatoms from the group consisting of oxygen, nitrogen and sulfur as ring members: for example mono- and bicyclic heterocycles having 7 ring members which, in addition to carbon ring members, contain one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.
a five- or six-membered aromatic (=maximum unsaturated) heterocycle (=heteroaromatic radical) which contains 1, 2 or 3 heteroatoms from the group consisting of oxygen, nitrogen and sulfur, for example 5-membered heteroaryl which is attached via carbon and contains one to three nitrogen atoms or one or two nitrogen atoms and one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl, which contains one, two or three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;

C₂-C₅-Alkylene: divalent branched or preferably unbranched chains having 2 to 5 carbon atoms, for example CH₂CH₂, —CH(CH₃)—, CH₂CH₂CH₂, CH(CH₃)CH₂, CH₂CH(CH₃), CH₂CH₂CH₂CH₂, CH₂CH₂CH₂CH₂CH₂.

C₄-C₅-Alkylene: divalent branched or preferably unbranched chains having 4 to 5 carbon atoms, for example CH₂CH₂CH₂CH₂ or CH₂CH₂CH₂CH₂CH₂.

The group —SM is more correctly spoken a group —S-M⁺, where M+ is a metal cation equivalent or an ammonium cation as defined above. A metal cation equivalent is more correctly spoken 1/a M^{a+}, where a is the valence of the metal and is in general 1, 2 or 3.

The protective group in the definition of R² may be any oxygen-protective group (to be more precise: an OH protective group) known in the art. OH groups, for instance, can be protected by means of a benzyl group, introduced by reaction with benzyl chloride for example; by a silyl protective group, for example trimethylsilyl (TMS), tert-butyldimethylsilyl (TBDMS) or tert-butyldiphenylsilyl (TBDPS), which is introduced by reaction with the corresponding chloride; by the tetrahydropyranyl protective group; by an alkyl group, such as C₁-C₆-alkyl; by a haloalkyl group, such as C₁-C₄-haloalkyl; by an alkenyl group, such as C₂-C₆-alkenyl; by a haloalkenyl group, such as C₂-C₄-haloalkenyl; by an alkylcarbonyl group, such as C₁-C₄-alkylcarbonyl; by a haloalkylcarbonyl group, such as C₁-C₄-haloalkylcarbonyl; by an alkoxycarbonyl protective group, such as C₁-C₄-alkoxycarbonyl; by a haloalkoxycarbonyl protective group, such as 2,2,2-trichloroethoxycarbonyl (TROC); or by an alkyl- or dialkylaminocarbonyl protective group, such as C₁-C₄-alkylaminocarbonyl or di-(C₁-C₄-alkyl)-aminocarbonyl.

The statements made below with respect to suitable and preferred features of the compounds according to the invention, especially with respect to their substituents R¹, R², R³, R⁴, R^{4a}, R⁵, R⁶, R⁷, R⁸, R⁹, R¹⁰, R¹¹, R¹², R¹³, R¹⁴, R¹⁵, R^{a}, R^{b}, R^{c}, R^{d}, Q, M and the index m, and to their use, are valid both per se and, in particular, in every possible combination with one another.

R¹ is preferably selected from C₁-C₆-alkyl, C₁-C₄-haloalkyl, C₃-C₆-cycloalkyl, C₃-C₆-halocycloalkyl, C₃-C₆-cycloalkyl-C₁-C₂-alkyl, C₃-C₆-halocycloalkyl-C₁-C₂-alkyl, where the cycloalkyl moiety in the 4 last-mentioned radicals may carry 1 or 2 substituents R⁶, where R⁶ is preferably selected from methyl, difluoromethyl and trifluoromethyl; phenyl which may carry 1, 2, 3, 4 or 5, preferably 1, 2 or 3 and in particular 1 or 2, substituents R⁵, and a 5- or 6-membered heteroaromatic ring containing 1, 2 or 3 heteroatoms selected from N, O and S as ring members, where the heteroaromatc ring may carry 1, 2 or 3 substituents R⁵. More preferably, R¹ is selected from C₁-C₆-alkyl, C₁-C₄-haloalkyl, C₃-C₆-cycloalkyl, C₃-C₆-halocycloalkyl, C₃-C₆-cycloalkyl-C₁-C₂-alkyl, C₃-C₆-halocycloalkyl-C₁-C₂-alkyl, where the cycloalkyl moiety in the 4 last-mentioned radicals may carry 1 substituent R⁶ selected from methyl, difluoromethyl and trifluoromethyl, and phenyl which may carry 1, 2, 3, 4 or 5, preferably 1, 2 or 3 and in particular 1 or 2, substituents R⁵.

Even more preferably, R¹ is selected from C₁-C₆-alkyl (preferably C₁-C₄-alkyl), cyclo-propyl, 1-methylcyclopropyl, 1-chlorocyclopropyl, 1-cyclopropylethyl and phenyl which may carry 1, 2, 3, 4 or 5, preferably 1, 2 or 3 and in particular 1 or 2, substituents R⁵ and particularly preferably from tert-butyl, cyclopropyl, 1-methylcyclopropyl, 1-chlorocyclopropyl, 1-cyclopropylethyl and phenyl. Specifically, R¹ is tert-butyl.

The protective group in the definition of R² is preferably selected from benzyl, a silyl protective group, such as trimethylsilyl (TMS), tert-butyldimethylsilyl (TBDMS) or tert-butyldiphenylsilyl (TBDPS), C₁-C₆-alkyl, C₁-C₄-haloalkyl, C₂-C₆-alkenyl, C₂-C₄-haloalkenyl, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylaminocarbonyl, and di-(C₁-C₄-alkyl)-aminocarbonyl. More preferably, the protective group in the definition of R² is selected from C₁-C₆-alkyl, C₁-C₄-haloalkyl, C₂-C₆-alkenyl, C₂-C₄-haloalkenyl, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylaminocarbonyl, and di-(C₁-C₄-alkyl)-aminocarbonyl.

R² is preferably selected from hydrogen and the above listed preferred and more preferred protective groups. More preferably, R² is hydrogen.

R³ is preferably selected from phenyl which may carry 1, 2 or 3 substituents R⁷, and a 5- or 6-membered heteroaromatic ring containing 1, 2 or 3 heteroatoms selected from N, O and S as ring members, where the heteroaromatic ring may carry 1, 2 or 3 substituents R⁸.

More preferably, R³ is phenyl which may carry 1, 2 or 3, preferably 1 or 2 substituents R⁷.

In a preferred embodiment, R⁷ is selected from C₁-C₄-alkyl, C₁-C₂-haloalkyl, C₂-C₄-alkenyl, C₁-C₄-alkoxy and C₁-C₂-haloalkoxy, and more preferably from C₁-C₃-alkyl, C₁-C₂-haloalkyl, C₂-C₃-alkenyl, C₁-C₃-alkoxy and C₁-C₂-haloalkoxy. Even more preferably, R⁷ is selected from methyl, trifluoromethyl, methoxy and trifluoromethoxy, particularly preferably from methyl, trifluoromethyl and methoxy and in particular from methyl and trifluoromethyl. Specifically, R⁷ is 4-methyl or 4-trifluoromethyl, relative to the 1-position of attachment point of the phenyl ring R³ to the remainder of the molecule.

In an alternatively preferred embodiment, R⁷ is selected from 2-chloro, 3-chloro, 2,4-dichloro, 3,4-dichloro, 2-chloro-4-fluoro, 2-fluoro-4-chloro, 3-chloro-4-fluoro, 3-fluoro-4-chloro, relative to the 1-position of attachment point of the phenyl ring R³ to the remainder of the molecule, and fluorine. Thus, R³, in an alternatively preferred embodiment of the invention is selected from 2-chlorophenyl, 3-chlorophenyl, 2,4-dichlorophenyl, 3,4-dichlorophenyl, 2-chloro-4-fluorophenyl, 2-fluoro-4-chlorophenyl, 3-chloro-4-fluorophenyl, 3-fluoro-4-chlorophenyl and phenyl carrying 1, 2 or 3 fluorine substituents. More preferably, R⁷ is selected from 2-chloro, 2,4-dichloro, 3,4-dichloro, 2-fluoro, 3-fluoro, 4-fluoro, 2,4-difluoro and 3,4-difluoro, relative to the 1-position of attachment point of the phenyl ring R³ to the remainder of the molecule. Thus, R³, in an alternatively more preferred embodiment of the invention is selected from 2-chlorophenyl, 2,4-dichlorophenyl, 3,4-dichlorophenyl, 2-fluorophenyl, 3-fluorophenyl, 4-fluorophenyl, 2,4-difluorophenyl and 3,4-difluorophenyl. Moreover, R⁷ is more preferably selected from 2-chloro-4-fluoro, 2-fluoro-4-chloro, 3-chloro-4-fluoro and 3-fluoro-4-chloro, relative to the 1-position of attachment point of the phenyl ring R³ to the remainder of the molecule. Thus, R³ is moreover preferably selected from 2-chloro-4-fluorophenyl, 2-fluoro-4-chlorophenyl, 3-chloro-4-fluorophenyl and 3-fluoro-4-chlorophenyl.

In yet another preferred embodiment of the invention, R⁷ is selected from 2-methyl, 3-methyl, 4-methyl, 2-trifluoromethyl, 3-trifluoromethyl, 4-trifluoromethyl, 2-methoxy, 3-methoxy, 4-methoxy, 2-trifluoromethoxy, 3-trifluoromethoxy, 4-trifluoromethoxy, 2-chloro, 3-chloro, 2,4-dichloro, 3,4-dichloro, 2-fluoro, 3-fluoro, 4-fluoro, 2,4-difluoro and 3,4-difluoro, and more preferably from 2-methyl, 3-methyl, 4-methyl, 2-trifluoromethyl, 3-trifluoromethyl, 4-trifluoromethyl, 2-methoxy, 3-methoxy, 4-methoxy, 2-trifluoromethoxy, 3-trifluoromethoxy, 4-trifluoromethoxy, 2-fluoro, 3-fluoro, 4-fluoro, 2,4-difluoro and 3,4-difluoro, relative to the 1-position of attachment point of the phenyl ring R³ to the remainder of the molecule. Thus, R³, in yet another preferred embodiment of the invention is selected from 2-methylphenyl, 3-methylphenyl, 4-methylphenyl, 2-trifluoromethylphenyl, 3-trifluoromethylphenyl, 4-trifluoromethylphenyl, 2-methoxyphenyl, 3-methoxyphenyl, 4-methoxyphenyl, 2-trifluoromethoxyphenyl, 3-trifluoromethoxyphenyl, 4-trifluoromethoxyphenyl, 2-chlorophenyl, 3-chlorophenyl, 2,4-dichlorophenyl, 3,4-dichlorophenyl, 2-fluorophenyl, 3-fluorophenyl, 4-fluorophenyl, 2,4-difluorophenyl and 3,4-difluorophenyl, and more preferably from 2-methylphenyl, 3-methylphenyl, 4-methylphenyl, 2-trifluoromethylphenyl, 3-trifluoromethylphenyl, 4-trifluoromethylphenyl, 2-methoxyphenyl, 3-methoxyphenyl, 4-methoxyphenyl, 2-trifluoromethoxyphenyl, 3-trifluoromethoxyphenyl, 4-trifluoromethoxyphenyl, 2-fluorophenyl, 3-fluorophenyl, 4-fluorophenyl, 2,4-difluorophenyl and 3,4-difluorophenyl, relative to the 1-position of attachment point of the phenyl ring R³ to the remainder of the molecule.

Preferably, R⁵, R⁸ and R⁹ are independently of each other and independently of each occurrence selected from halogen, C₁-C₄-alkyl, C₁-C₄-haloalkyl, C₁-C₄-alkoxy and C₁-C₄-haloalkoxy and more preferably from F, Cl, methyl, difluoromethyl, trifluoromethyl, methoxy, difluoromethoxy and trifluoromethoxy.

Preferably, R⁶ is independently of each occurrence selected from C₁-C₄-alkyl, C₁-C₄-haloalkyl, C₁-C₄-alkoxy and C₁-C₄-haloalkoxy and more preferably from methyl, di-fluoromethyl, trifluoromethyl, methoxy, difluoromethoxy and trifluoromethoxy.

R¹⁰ in the groups —C(═O)R¹⁰ and —S(O)₂R¹⁰ is preferably selected from C₁-C₄-alkyl, C₁-C₂-haloalkyl, C₁-C₄-alkoxy, C₁-C₂-haloalkoxy, phenyl, phenoxy and NR¹³R¹⁴, more preferably from C₁-C₄-alkyl, C₁-C₂-haloalkyl, C₁-C₄-alkoxy, C₁-C₂-haloalkoxy and NR¹³R¹⁴ and even more preferably from C₁-C₄-alkyl, C₁-C₄-alkoxy and NR¹³R¹⁴. In the group —C(═O)R¹⁰, R¹⁰ is specifically C₁-C₄-alkyl, such as methyl, ethyl, propyl, isopropyl, n-butyl, sec-butyl, isobutyl or tert-butyl, preferably methyl, or is C₁-C₄-alkoxy, such as methoxy, ethoxy, propoxy, isopropoxy, n-butoxy, sec-butoxy, isobutoxy or tert-butoxy, preferably methoxy, and is more specifically methyl, and in the group —S(O)₂R¹⁰, R¹⁰ is specifically methyl. Preferably, R¹³ is hydrogen and R¹⁴ is selected from hydrogen, C₁-C₄-alkyl and phenyl, preferably from hydrogen and C₁-C₄-alkyl, or the two of R¹³ and R¹⁴ are C₁-C₄-alkyl.

R⁴ is preferably selected from hydrogen, C₁-C₄-alkyl, phenyl, 4-methylphenyl, —C(═O)R¹⁰, —S(O)₂R¹⁰, —CN, M and a group of the formula III, where R¹⁰ has one of the above general meanings or, in particular, one of the above preferred meanings and M has one of the above general meanings or, in particular, one of the below-given preferred meanings.

R⁴ is more preferably selected from hydrogen, C₁-C₄-alkyl, —C(═O)R¹⁰, —S(O)₂R¹⁰, —CN, M and a group of the formula III, where R¹⁰ has one of the above general meanings or, in particular, one of the above preferred meanings and M has one of the above general meanings or, in particular, one of the below-given preferred meanings.

R⁴ is even more preferably selected from hydrogen, C₁-C₄-alkyl, C₃-C₄-alkylcarbonyl, C₁-C₄-alkoxycarbonyl, —C(═O)N(H)C₁-C₄-alkyl, —C(═O)N(C₁-C₄-alkyl)₂, C₁-C₄-alkylsulfonyl, CN and a group of the formula III. In particular, R⁴ is selected from hydrogen, CN, methylcarbonyl, methoxycarbonyl and methyl. Specifically, R⁴ is hydrogen.

M is preferably selected from an alkali metal cation, an earth alkaline metal cation equivalent, a cation equivalent of Cu, Zn, Fe or Ni or an ammonium cation of formula (NR^{a}R^{b}R^{c}R^{d})⁺, wherein one of R^{a}, R^{b}, R^{c} and R^{d} is hydrogen and three of R^{a}, R^{b}, R^{c} and R^{d}, independently of each other, are selected from C₁-C₁₀-alkyl. More preferably, M is selected from Li⁺, Na⁺, K⁺, ½Mg²⁺, a cation equivalent of Cu, Zn, Fe or Ni and an ammonium cation of formula (NR^{a}R^{b}R^{c}R^{d})⁺, wherein one of R^{a}, R^{b}, R^{c} and R^{d} is hydrogen and three of R^{a}, R^{b}, R^{c} and R^{d}, independently of each other, are selected from C₁-C₁₀-alkyl. Even more preferably, M is selected from Na⁺, K⁺, ½Mg²⁺, ½Cu²⁺, ½Zn²⁺, ½Fe²⁺, ½Ni²⁺, triethylammonium and trimethylammonium.

In the group of formula III, the variables preferably have the same meanings as in the remainder of the molecule I. Thus, the remarks made above as to preferred meanings of the radicals apply to this moiety, too.

R^{4a} is preferably selected from hydrogen, C₁-C₁₀-alkyl, C₁-C₄-haloalkyl, phenyl, 4-methylphenyl, phenyl-C₁-C₄-alkyl, —C(═O)R¹⁰ and —S(O)₂R¹⁰, where R¹⁰ has one of the above given general or, in particular, one of the above-given preferred meanings. More preferably, R^{4a} is selected from hydrogen, C₁-C₄-alkyl, C₁-C₄-haloalkyl, phenyl, benzyl, —C(═O)R¹⁰ and —S(O)₂R¹⁰, where R¹⁰ has one of the above given general or, in particular, one of the above-given preferred meanings, and more preferably from hydrogen, C₁-C₄-alkyl, C₁-C₄-haloalkyl, —C(═O)R¹⁰ and —S(O)₂R¹⁰, where R¹⁰ has one of the above given general or, in particular, one of the above-given preferred meanings. In particular, R^{4a} is hydrogen, C₁-C₄-alkyl, preferably methyl, or —C(═O)R¹⁰, more particularly hydrogen, C₁-C₄-alkyl, preferably methyl, methylcarbonyl or methoxycarbonyl, even more particularly hydrogen or C₁-C₄-alkyl, preferably methyl, and is specifically hydrogen.

If m is 1, the oxygen atom is preferably bound via a double bond to the sulfur atom, the radical —S(O)ₘ—R⁴ thus resulting in a group —S(═O)—R⁴. If m is 2, the two oxygen atoms are preferably both bound via a double bond to the sulfur atom, the radical —S(O)ₘ—R⁴ thus resulting in a group —S(═O)₂—R⁴. If m is 3, the radical —S(O)ₘ—R⁴ is a group —S(═O)₂—O—R⁴.

m is preferably 0.

In a particularly preferred embodiment, in compounds I, m is 0 and R⁴ is H (or, alternatively, in compounds II, R^{4a} is H).

Particularly preferred compounds I are compounds of formula I.A

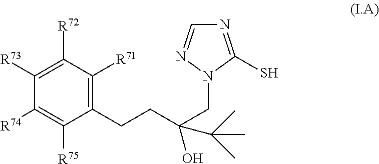

wherein R⁷¹, R⁷², R⁷³, R⁷⁴ and R⁷⁵ are hydrogen or have one of the general or, in particular, one of the preferred meanings given for R⁷.

Preferably, in compounds I.A the combination of R⁷¹, R⁷², R⁷³, R⁷⁴ and R⁷⁵ is as given in following table:

| Compound | R⁷¹ | R⁷² | R⁷³ | R⁷⁴ | R⁷⁵ |
|---|---|---|---|---|---|
| I.A.1 | H | H | H | H | H |
| I.A.2 | F | H | H | H | H |
| I.A.3 | H | F | H | H | H |
| I.A.4 | H | H | F | H | H |
| I.A.5 | F | F | H | H | H |
| I.A.6 | F | H | F | H | H |
| I.A.7 | F | H | H | F | H |
| I.A.8 | F | H | H | H | F |
| I.A.9 | H | F | F | H | H |
| I.A.10 | H | F | H | F | H |
| I.A.11 | F | H | F | H | F |
| I.A.12 | Cl | H | H | H | H |
| I.A.13 | H | Cl | H | H | H |
| I.A.14 | Cl | Cl | H | H | H |
| I.A.15 | Cl | H | Cl | H | H |
| I.A.16 | Cl | H | H | Cl | H |
| I.A.17 | Cl | H | H | H | Cl |
| I.A.18 | H | Cl | Cl | H | H |
| I.A.19 | H | Cl | H | Cl | H |
| I.A.20 | Cl | H | Cl | H | Cl |
| I.A.21 | F | Cl | H | H | H |
| I.A.22 | F | H | Cl | H | H |
| I.A.23 | F | H | H | Cl | H |
| I.A.24 | F | H | H | H | Cl |
| I.A.25 | H | F | Cl | H | H |
| I.A.26 | H | F | H | Cl | H |
| I.A.27 | Cl | F | H | H | H |
| I.A.28 | Cl | H | F | H | H |
| I.A.29 | Cl | H | H | F | H |
| I.A.30 | H | Cl | F | H | H |
| I.A.31 | CH₃ | H | H | H | H |
| I.A.32 | H | CH₃ | H | H | H |
| I.A.33 | H | H | CH₃ | H | H |
| I.A.34 | CH₃ | CH₃ | H | H | H |
| I.A.35 | CH₃ | H | CH₃ | H | H |
| I.A.36 | CH₃ | H | H | CH₃ | H |
| I.A.37 | CH₃ | H | H | H | CH₃ |
| I.A.38 | CH₃ | H | CH₃ | H | CH₃ |
| I.A.39 | CF₃ | H | H | H | H |
| I.A.40 | H | CF₃ | H | H | H |
| I.A.41 | H | H | CF₃ | H | H |
| I.A.42 | CF₃ | CF₃ | H | H | H |
| I.A.43 | CF₃ | H | CF₃ | H | H |
| I.A.44 | CF₃ | H | H | CF₃ | H |
| I.A.45 | CF₃ | H | H | H | CF₃ |
| I.A.46 | CF₃ | H | CF₃ | H | CF₃ |

Particular compounds I/II/I.A are the following:
3-(5-mercapto-[1,2,4]-triazol-1-ylmethyl)-2,2-dimethyl-6-phenyl-pentan-3-ol (compound I.A.1),
3-(5-mercapto-[1,2,4]-triazol-1-ylmethyl)-2,2-dimethyl-6-(2-fluorophenyl)-pentan-3-ol (compound I.A.2);
3-(5-mercapto-[1,2,4]-triazol-1-ylmethyl)-2,2-dimethyl-6-(4-fluorophenyl)-pentan-3-ol (compound I.A.4);
3-(5-mercapto-[1,2,4]-triazol-1-ylmethyl)-2,2-dimethyl-6-(2,4-difluorophenyl)-pentan-3-ol (compound I.A.6);
3-(5-mercapto-[1,2,4]-triazol-1-ylmethyl)-2,2-dimethyl-6-(3,4-difluorophenyl)-pentan-3-ol (compound I.A.9);
3-(5-mercapto-[1,2,4]-triazol-1-ylmethyl)-2,2-dimethyl-6-(2-chlorophenyl)-pentan-3-ol (compound I.A.12);
3-(5-mercapto-[1,2,4]-triazol-1-ylmethyl)-2,2-dimethyl-6-(3-chlorophenyl)-pentan-3-ol (compound I.A.13);
3-(5-mercapto-[1,2,4]-triazol-1-ylmethyl)-2,2-dimethyl-6-(2,4-dichlorophenyl)-pentan-3-ol (compound I.A.15);
3-(5-mercapto-[1,2,4]-triazol-1-ylmethyl)-2,2-dimethyl-6-(3,4-dichlorophenyl)-pentan-3-ol (compound I.A.18);
3-(5-mercapto-[1,2,4]-triazol-1-ylmethyl)-2,2-dimethyl-6-(4-(trifluoromethyl)-phenyl)-pentan-3-ol (compound I.A.41);
3-(5-mercapto-[1,2,4]-triazol-1-ylmethyl)-2,2-dimethyl-6-(4-methylphenyl)-pentan-3-ol (compound I.A.33).

Examples for preferred compounds I and II are compounds of formulae I.1, I.2 and II.1, where the variables have one of the general or, in particular, one of the preferred meanings given above. Examples of preferred compounds are the individual compounds compiled in the tables 1 to 522 below. Moreover, the meanings mentioned below for the individual variables in the tables are per se, independently of the combination in which they are mentioned, a particularly preferred embodiment of the substituents in question.

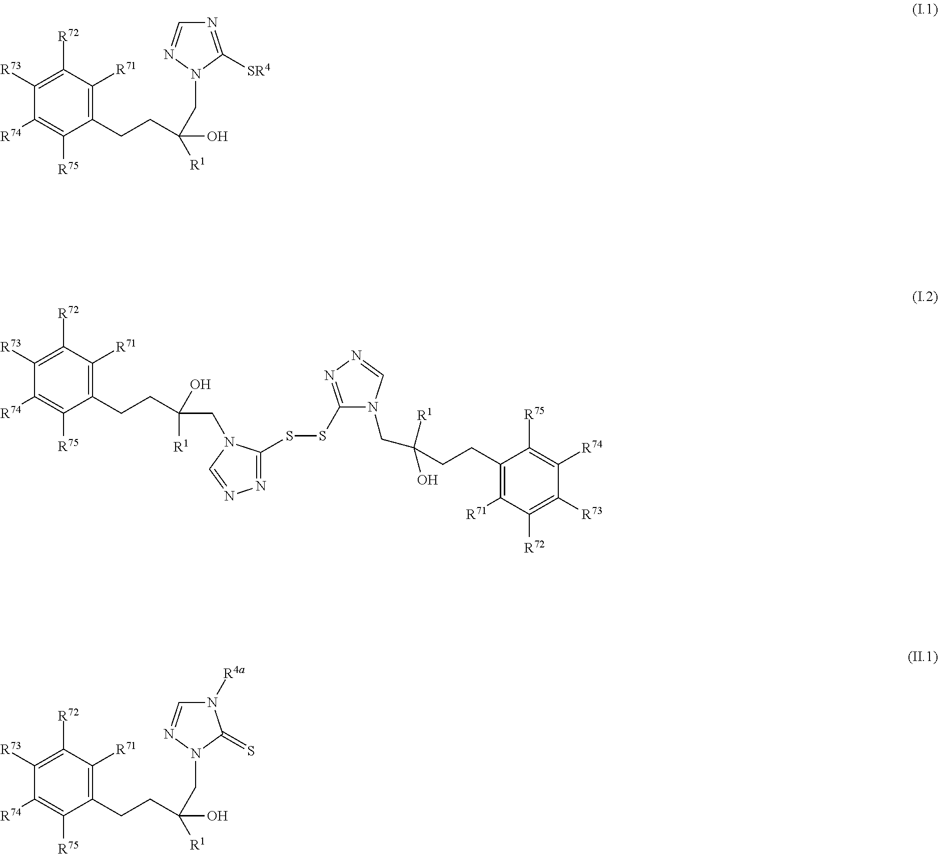

### Table 1

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is H

### Table 2

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methyl

### Table 3

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethyl

### Table 4

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propyl

### Table 5

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isopropyl

### Table 6

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is n-butyl

### Table 7

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is sec-butyl

### Table 8

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isobutyl

### Table 9

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is tert-butyl

### Table 10

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is phenyl

### Table 11

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is 4-methyl phenyl

### Table 12

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is Li⁺

### Table 13

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is Na⁺

### Table 14

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is K⁺

### Table 15

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ½Mg²⁺

### Table 16

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ½Cu²⁺

### Table 17

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ½Zn²⁺

### Table 18

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ½Fe²⁺

### Table 19

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ½Ni²⁺

### Table 20

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is NH(CH₃)₃⁺

### Table 21

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is NH(C₂H₅)₃⁺

### Table 22

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is NH(CH₂CH₂CH₂)₃⁺

### Table 23

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is NH(CH(CH₃)₂)₃⁺

### Table 24

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is NH(CH₂CH₂CH₂CH₂)₃⁺

### Table 25

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methylcarbonyl

### Table 26

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethylcarbonyl

### Table 27

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propylcarbonyl

### Table 28

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isopropylcarbonyl

### Table 29

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methoxycarbonyl

### Table 30

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethoxycarbonyl

### Table 31

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propoxycarbonyl

### Table 32

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isopropoxycarbonyl

### Table 33

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is phenoxycarbonyl

### Table 34

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methylaminocarbonyl

### Table 35

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethylaminocarbonyl

### Table 36

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propylaminocarbonyl

### Table 37

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isopropylaminocarbonyl

### Table 38

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is phenylaminocarbonyl

### Table 39

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methylsulfonyl

### Table 40

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethylsulfonyl

### Table 41

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propylsulfonyl

### Table 42

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is isopropylsulfonyl

### Table 43

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is phenylsulfonyl

### Table 44

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is methoxysulfonyl

### Table 45

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is ethoxysulfonyl

### Table 46

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is propoxysulfonyl

### Table 47

Compounds of the formula I.1 in which the combination of R⁹¹, R⁹², R⁹³, R⁹⁴ and R⁹⁵ for a compound corresponds in each case to one row of Table A, R¹ is tert-butyl and R⁴ is isopropoxysulfonyl

### Table 48

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is phenoxysulfonyl

### Table 49

Compounds of the formula I.1 in which the combination of R⁷¹, R⁷², R⁷³, R⁷⁴ and R⁷⁵ for a compound corresponds in each case to one row of Table A, R¹ is tert-butyl and R⁴ is CN

### Tables 50 to 98

Compounds of the formula I.1 in which the combination of R⁷¹, R⁷², R⁷³, R⁷⁴ and R⁷⁵ for a compound corresponds in each case to one row of Table A, R⁴ is as defined in any of tables 1 to 49 and R¹ is cyclopropyl

### Tables 99 to 147

Compounds of the formula I.1 in which the combination of R⁷¹, R⁷², R⁷³, R⁷⁴ and R⁷⁵ for a compound corresponds in each case to one row of Table A, R⁴ is as defined in any of tables 1 to 49 and R¹ is 1-methylcyclopropyl

### Tables 148 to 196

Compounds of the formula I.1 in which the combination of R⁷¹, R⁷², R⁷³, R⁷⁴ and R⁷⁵ for a compound corresponds in each case to one row of Table A, R⁴ is as defined in any of tables 1 to 49 and R¹ is 1-chlorocyclopropyl

### Tables 197 to 245

Compounds of the formula I.1 in which the combination of R⁷¹, R⁷², R⁷³, R⁷⁴ and R⁷⁵ for a compound corresponds in each case to one row of Table A, R⁴ is as defined in any of tables 1 to 49 and R¹ is cyclopropylmethyl

### Tables 246 to 294

Compounds of the formula I.1 in which the combination of R⁷¹, R⁷², R⁷³, R⁷⁴ and R⁷⁵ for a compound corresponds in each case to one row of Table A, R⁴ is as defined in any of tables 1 to 49 and R¹ is 1-cyclopropylethyl

### Table 295

Compounds of the formula I.2 in which the combination of R⁷¹, R⁷², R⁷³, R⁷⁴ and R⁷⁵ for a compound corresponds in each case to one row of Table A and R¹ is tert-butyl

### Table 296

Compounds of the formula I.2 in which the combination of R⁷¹, R⁷², R⁷³, R⁷⁴ and R⁷⁵ for a compound corresponds in each case to one row of Table A and R¹ is cyclopropyl

### Table 297

Compounds of the formula I.2 in which the combination of R⁷¹, R⁷², R⁷³, R⁷⁴ and R⁷⁵ for a compound corresponds in each case to one row of Table A and R¹ is 1-methylcyclopropyl

### Table 298

Compounds of the formula I.2 in which the combination of R⁷¹, R⁷², R⁷³, R⁷⁴ and R⁷⁵ for a compound corresponds in each case to one row of Table A and R¹ is 1-chlorocyclopropyl

### Table 299

Compounds of the formula I.2 in which the combination of R⁷¹, R⁷², R⁷³, R⁷⁴ and R⁷⁵ for a compound corresponds in each case to one row of Table A and R¹ is cyclopropyl-methyl

### Table 300

Compounds of the formula I.2 in which the combination of R⁷¹, R⁷², R⁷³, R⁷⁴ and R⁷⁵ for a compound corresponds in each case to one row of Table A and R¹ is 1-cyclopropylethyl

### Table 301

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is H

### Table 302

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methyl

### Table 303

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethyl

### Table 304

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is n-propyl

### Table 305

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropyl

### Table 306

Compounds of the formula II.1 in which the combination of R⁹¹, R⁹², R⁹³, R⁹⁴ and R⁹⁵ for a compound corresponds in each case to one row of Table A, R¹ is tert-butyl and R^{4a} is n-butyl

### Table 307

Compounds of the formula II.1 in which the combination of R⁹¹, R⁹², R⁹³, R⁹⁴ and R⁹⁵ for a compound corresponds in each case to one row of Table A, R¹ is tert-butyl and R^{4a} is sec-butyl

### Table 308

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isobutyl

### Table 309

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is tert-butyl

### Table 310

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenyl

### Table 311

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is 4-methyl phenyl

### Table 312

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methylcarbonyl

### Table 313

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethylcarbonyl

### Table 314

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is propylcarbonyl

### Table 315

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropylcarbonyl

### Table 316

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenylcarbonyl

### Table 317

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methoxycarbonyl

### Table 318

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethoxycarbonyl

### Table 319

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is propoxycarbonyl

### Table 320

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropoxycarbonyl

### Table 321

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenoxycarbonyl

### Table 322

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methylaminocarbonyl

### Table 323

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethylaminocarbonyl

### Table 324

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is propylaminocarbonyl

### Table 325

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropylaminocarbonyl

### Table 326

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenylaminocarbonyl

### Table 327

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methylsulfonyl

### Table 328

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethylsulfonyl

### Table 329

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is propylsulfonyl

### Table 330

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropylsulfonyl

### Table 331

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenylsulfonyl

### Table 332

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is methoxysulfonyl

### Table 333

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is ethoxysulfonyl

### Table 334

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is propoxysulfonyl

### Table 335

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is isopropoxysulfonyl

### Table 336

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is phenoxysulfonyl

### Table 337

Compounds of the formula II.1 in which the combination of R⁷¹, R⁷², R⁷³, R⁷⁴ and R⁷⁵ for a compound corresponds in each case to one row of Table A, R¹ is tert-butyl and R^{4a} is CN

### Tables 338 to 374

Compounds of the formula II.1 in which the combination of R⁷¹, R⁷², R⁷³, R⁷⁴ and R⁷⁵ for a compound corresponds in each case to one row of Table A, R^{4a} is as defined in any of tables 301 to 337 and R¹ is cyclopropyl

### Tables 375 to 411

Compounds of the formula II.1 in which the combination of R⁷¹, R⁷², R⁷³, R⁷⁴ and R⁷⁵ for a compound corresponds in each case to one row of Table A, R^{4a} is as defined in any of tables 301 to 337 and R¹ is 1-methylcyclopropyl

### Tables 412 to 448

Compounds of the formula II.1 in which the combination of R⁷¹, R⁷², R⁷³, R⁷⁴ and R⁷⁵ for a compound corresponds in each case to one row of Table A, R^{4a} is as defined in any of tables 301 to 337 and R¹ is 1-chlorocyclopropyl

### Tables 449 to 485

Compounds of the formula II.1 in which the combination of R⁷¹, R⁷², R⁷³, R⁷⁴ and R⁷⁵ for a compound corresponds in each case to one row of Table A, R^{4a} is as defined in any of tables 301 to 337 and R¹ is cyclopropylmethyl

### Tables 486 to 522

Compounds of the formula II.1 in which the combination of R⁷¹, R⁷², R⁷³, R⁷⁴ and R⁷⁵ for a compound corresponds in each case to one row of Table A, R^{4a} is as defined in any of tables 301 to 337 and R¹ is 1-cyclopropylethyl

TABLE A

| No. | R⁷¹ | R⁷² | R⁷³ | R⁷⁴ | R⁷⁵ |
|---|---|---|---|---|---|
| A-1 | H | H | H | H | H |
| A-2 | F | H | H | H | H |
| A-3 | H | F | H | H | H |
| A-4 | H | H | F | H | H |
| A-5 | Cl | H | H | H | H |
| A-6 | H | Cl | H | H | H |
| A-7 | Br | H | H | H | H |
| A-8 | H | Br | H | H | H |
| A-9 | H | H | Br | H | H |
| A-10 | CH₃ | H | H | H | H |
| A-11 | H | CH₃ | H | H | H |
| A-12 | H | H | CH₃ | H | H |
| A-13 | CHF₂ | H | H | H | H |
| A-14 | H | CHF₂ | H | H | H |
| A-15 | H | H | CHF₂ | H | H |
| A-16 | CF₃ | H | H | H | H |
| A-17 | H | CF₃ | H | H | H |
| A-18 | H | H | CF₃ | H | H |
| A-19 | OCH₃ | H | H | H | H |
| A-20 | H | OCH₃ | H | H | H |
| A-21 | H | H | OCH₃ | H | H |
| A-22 | OCHF₂ | H | H | H | H |
| A-23 | H | OCHF₂ | H | H | H |
| A-24 | H | H | OCHF₂ | H | H |
| A-25 | OCF₃ | H | H | H | H |
| A-26 | H | OCF₃ | H | H | H |
| A-27 | H | H | OCF₃ | H | H |
| A-28 | F | F | H | H | H |
| A-29 | F | H | F | H | H |
| A-30 | F | H | H | F | H |
| A-31 | F | H | H | H | F |
| A-32 | H | F | F | H | H |
| A-33 | H | F | H | F | H |
| A-34 | Cl | Cl | H | H | H |
| A-35 | Cl | H | Cl | H | H |
| A-36 | Cl | H | H | Cl | H |
| A-37 | Cl | H | H | H | Cl |
| A-38 | H | Cl | Cl | H | H |
| A-39 | H | Cl | H | Cl | H |
| A-40 | Br | Br | H | H | H |
| A-41 | Br | H | Br | H | H |
| A-42 | Br | H | H | Br | H |
| A-43 | Br | H | H | H | Br |
| A-44 | H | Br | Br | H | H |
| A-45 | H | Br | H | Br | H |
| A-46 | CH₃ | CH₃ | H | H | H |
| A-47 | CH₃ | H | CH₃ | H | H |
| A-48 | CH₃ | H | H | CH₃ | H |
| A-49 | CH₃ | H | H | H | CH₃ |
| A-50 | H | CH₃ | CH₃ | H | H |
| A-51 | H | CH₃ | H | CH₃ | H |
| A-52 | CHF₂ | CHF₂ | H | H | H |
| A-53 | CHF₂ | H | CHF₂ | H | H |
| A-54 | CHF₂ | H | H | CHF₂ | H |
| A-55 | CHF₂ | H | H | H | CHF₂ |
| A-56 | H | CHF₂ | CHF₂ | H | H |
| A-57 | H | CHF₂ | H | CHF₂ | H |
| A-58 | CF₃ | CF₃ | H | H | H |
| A-59 | CF₃ | H | CF₃ | H | H |
| A-60 | CF₃ | H | H | CF₃ | H |
| A-61 | CF₃ | H | H | H | CF₃ |
| A-62 | H | CF₃ | CF₃ | H | H |
| A-63 | H | CF₃ | H | CF₃ | H |
| A-64 | OCH₃ | OCH₃ | H | H | H |
| A-65 | OCH₃ | H | OCH₃ | H | H |
| A-66 | OCH₃ | H | H | OCH₃ | H |
| A-67 | OCH₃ | H | H | H | OCH₃ |
| A-68 | H | OCH₃ | OCH₃ | H | H |
| A-69 | H | OCH₃ | H | OCH₃ | H |
| A-70 | OCHF₂ | OCHF₂ | H | H | H |
| A-71 | OCHF₂ | H | OCHF₂ | H | H |
| A-72 | OCHF₂ | H | H | OCHF₂ | H |
| A-73 | OCHF₂ | H | H | H | OCHF₂ |
| A-74 | H | OCHF₂ | OCHF₂ | H | H |
| A-75 | H | OCHF₂ | H | OCHF₂ | H |
| A-76 | OCF₃ | OCF₃ | H | H | H |
| A-77 | OCF₃ | H | OCF₃ | H | H |
| A-78 | OCF₃ | H | H | OCF₃ | H |
| A-79 | OCF₃ | H | H | H | OCF₃ |
| A-80 | H | OCF₃ | OCF₃ | H | H |
| A-81 | H | OCF₃ | H | OCF₃ | H |
| A-82 | F | Cl | H | H | H |
| A-83 | F | H | Cl | H | H |
| A-84 | F | H | H | Cl | H |
| A-85 | F | H | H | H | Cl |
| A-86 | H | F | Cl | H | H |
| A-87 | H | F | H | Cl | H |
| A-88 | Cl | F | H | H | H |
| A-89 | Cl | H | F | H | H |
| A-90 | Cl | H | H | F | H |
| A-91 | H | Cl | F | H | H |
| A-92 | F | Br | H | H | H |
| A-93 | F | H | Br | H | H |
| A-94 | F | H | H | Br | H |
| A-95 | F | H | H | H | Br |
| A-96 | H | F | Br | H | H |
| A-97 | H | F | H | Br | H |
| A-98 | Br | F | H | H | H |
| A-99 | Br | H | F | H | H |
| A-100 | Br | H | H | F | H |
| A-101 | H | Br | F | H | H |
| A-102 | F | CH₃ | H | H | H |
| A-103 | F | H | CH₃ | H | H |
| A-104 | F | H | H | CH₃ | H |
| A-105 | F | H | H | H | CH₃ |
| A-106 | H | F | CH₃ | H | H |
| A-107 | H | F | H | CH₃ | H |
| A-108 | CH₃ | F | H | H | H |
| A-109 | CH₃ | H | F | H | H |
| A-110 | CH₃ | H | H | F | H |
| A-111 | H | CH₃ | F | H | H |
| A-112 | F | CHF₂ | H | H | H |
| A-113 | F | H | CHF₂ | H | H |
| A-114 | F | H | H | CHF₂ | H |
| A-115 | F | H | H | H | CHF₂ |
| A-116 | H | F | CHF₂ | H | H |
| A-117 | H | F | H | CHF₂ | H |
| A-118 | CHF₂ | F | H | H | H |
| A-119 | CHF₂ | H | F | H | H |
| A-120 | CHF₂ | H | H | F | H |
| A-121 | H | CHF₂ | F | H | H |
| A-122 | F | CF₃ | H | H | H |
| A-123 | F | H | CF₃ | H | H |
| A-124 | F | H | H | CF₃ | H |
| A-125 | F | H | H | H | CF₃ |
| A-126 | H | F | CF₃ | H | H |
| A-127 | H | F | H | CF₃ | H |
| A-128 | CF₃ | F | H | H | H |
| A-129 | CF₃ | H | F | H | H |
| A-130 | CF₃ | H | H | F | H |
| A-131 | H | CF₃ | F | H | H |
| A-132 | F | OCH₃ | H | H | H |
| A-133 | F | H | OCH₃ | H | H |
| A-134 | F | H | H | OCH₃ | H |
| A-135 | F | H | H | H | OCH₃ |
| A-136 | H | F | OCH₃ | H | H |
| A-137 | H | F | H | OCH₃ | H |
| A-138 | OCH₃ | F | H | H | H |
| A-139 | OCH₃ | H | F | H | H |
| A-140 | OCH₃ | H | H | F | H |
| A-141 | H | OCH₃ | F | H | H |
| A-142 | F | OCHF₂ | H | H | H |
| A-143 | F | H | OCHF₂ | H | H |
| A-144 | F | H | H | OCHF₂ | H |
| A-145 | F | H | H | H | OCHF₂ |
| A-146 | H | F | OCHF₂ | H | H |
| A-147 | H | F | H | OCHF₂ | H |
| A-148 | OCHF₂ | F | H | H | H |
| A-149 | OCHF₂ | H | F | H | H |
| A-150 | OCHF₂ | H | H | F | H |
| A-151 | H | OCHF₂ | F | H | H |
| A-152 | F | OCF₃ | H | H | H |
| A-153 | F | H | OCF₃ | H | H |
| A-154 | F | H | H | OCF₃ | H |
| A-155 | F | H | H | H | OCF₃ |
| A-156 | H | F | OCF₃ | H | H |
| A-157 | H | F | H | OCF₃ | H |
| A-158 | OCF₃ | F | H | H | H |
| A-159 | OCF₃ | H | F | H | H |
| A-160 | OCF₃ | H | H | F | H |
| A-161 | H | OCF₃ | F | H | H |
| A-162 | Cl | Br | H | H | H |
| A-163 | Cl | H | Br | H | H |
| A-164 | Cl | H | H | Br | H |
| A-165 | Cl | H | H | H | Br |
| A-166 | H | Cl | Br | H | H |
| A-167 | H | Cl | H | Br | H |
| A-168 | Br | Cl | H | H | H |
| A-169 | Br | H | Cl | H | H |
| A-170 | Br | H | H | Cl | H |
| A-171 | H | Br | Cl | H | H |
| A-172 | Cl | CH₃ | H | H | H |
| A-173 | Cl | H | CH₃ | H | H |
| A-174 | Cl | H | H | CH₃ | H |
| A-175 | Cl | H | H | H | CH₃ |
| A-176 | H | Cl | CH₃ | H | H |
| A-177 | H | Cl | H | CH₃ | H |
| A-178 | CH₃ | Cl | H | H | H |
| A-179 | CH₃ | H | Cl | H | H |
| A-180 | CH₃ | H | H | Cl | H |
| A-181 | H | CH₃ | Cl | H | H |
| A-182 | Cl | CHF₂ | H | H | H |
| A-183 | Cl | H | CHF₂ | H | H |
| A-184 | Cl | H | H | CHF₂ | H |
| A-185 | Cl | H | H | H | CHF₂ |
| A-186 | H | Cl | CHF₂ | H | H |
| A-187 | H | Cl | H | CHF₂ | H |
| A-188 | CHF₂ | Cl | H | H | H |
| A-189 | CHF₂ | H | Cl | H | H |
| A-190 | CHF₂ | H | H | Cl | H |
| A-191 | H | CHF₂ | Cl | H | H |
| A-192 | Cl | CF₃ | H | H | H |
| A-193 | Cl | H | CF₃ | H | H |
| A-194 | Cl | H | H | CF₃ | H |
| A-195 | Cl | H | H | H | CF₃ |
| A-196 | H | Cl | CF₃ | H | H |
| A-197 | H | Cl | H | CF₃ | H |
| A-198 | CF₃ | Cl | H | H | H |
| A-199 | CF₃ | H | Cl | H | H |
| A-200 | CF₃ | H | H | Cl | H |
| A-201 | H | CF₃ | Cl | H | H |
| A-202 | Cl | OCH₃ | H | H | H |
| A-203 | Cl | H | OCH₃ | H | H |
| A-204 | Cl | H | H | OCH₃ | H |
| A-205 | Cl | H | H | H | OCH₃ |
| A-206 | H | Cl | OCH₃ | H | H |
| A-207 | H | Cl | H | OCH₃ | H |
| A-208 | OCH₃ | Cl | H | H | H |
| A-209 | OCH₃ | H | Cl | H | H |
| A-210 | OCH₃ | H | H | Cl | H |
| A-211 | H | OCH₃ | Cl | H | H |
| A-212 | Cl | OCHF₂ | H | H | H |
| A-213 | Cl | H | OCHF₂ | H | H |
| A-214 | Cl | H | H | OCHF₂ | H |
| A-215 | Cl | H | H | H | OCHF₂ |
| A-216 | H | Cl | OCHF₂ | H | H |
| A-217 | H | Cl | H | OCHF₂ | H |
| A-218 | OCHF₂ | Cl | H | H | H |
| A-219 | OCHF₂ | H | Cl | H | H |
| A-220 | OCHF₂ | H | H | Cl | H |
| A-221 | H | OCHF₂ | Cl | H | H |
| A-222 | Cl | OCF₃ | H | H | H |
| A-223 | Cl | H | OCF₃ | H | H |
| A-224 | Cl | H | H | OCF₃ | H |
| A-225 | Cl | H | H | H | OCF₃ |
| A-226 | H | Cl | OCF₃ | H | H |
| A-227 | H | Cl | H | OCF₃ | H |
| A-228 | OCF₃ | Cl | H | H | H |
| A-229 | OCF₃ | H | Cl | H | H |
| A-230 | OCF₃ | H | H | Cl | H |
| A-231 | H | OCF₃ | Cl | H | H |
| A-232 | Br | CH₃ | H | H | H |
| A-233 | Br | H | CH₃ | H | H |
| A-234 | Br | H | H | CH₃ | H |
| A-235 | Br | H | H | H | CH₃ |
| A-236 | H | Br | CH₃ | H | H |
| A-237 | H | Br | H | CH₃ | H |
| A-238 | CH₃ | Br | H | H | H |
| A-239 | CH₃ | H | Br | H | H |
| A-240 | CH₃ | H | H | Br | H |
| A-241 | H | CH₃ | Br | H | H |
| A-242 | Br | CHF₂ | H | H | H |
| A-243 | Br | H | CHF₂ | H | H |
| A-244 | Br | H | H | CHF₂ | H |
| A-245 | Br | H | H | H | CHF₂ |
| A-246 | H | Br | CHF₂ | H | H |
| A-247 | H | Br | H | CHF₂ | H |
| A-248 | CHF₂ | Br | H | H | H |
| A-249 | CHF₂ | H | Br | H | H |
| A-250 | CHF₂ | H | H | Br | H |
| A-251 | H | CHF₂ | Br | H | H |
| A-252 | Br | CF₃ | H | H | H |
| A-253 | Br | H | CF₃ | H | H |
| A-254 | Br | H | H | CF₃ | H |
| A-255 | Br | H | H | H | CF₃ |
| A-256 | H | Br | CF₃ | H | H |
| A-257 | H | Br | H | CF₃ | H |
| A-258 | CF₃ | Br | H | H | H |
| A-259 | CF₃ | H | Br | H | H |
| A-260 | CF₃ | H | H | Br | H |
| A-261 | H | CF₃ | Br | H | H |
| A-262 | Br | OCH₃ | H | H | H |
| A-263 | Br | H | OCH₃ | H | H |
| A-264 | Br | H | H | OCH₃ | H |
| A-265 | Br | H | H | H | OCH₃ |
| A-266 | H | Br | OCH₃ | H | H |
| A-267 | H | Br | H | OCH₃ | H |
| A-268 | OCH₃ | Br | H | H | H |
| A-269 | OCH₃ | H | Br | H | H |
| A-270 | OCH₃ | H | H | Br | H |
| A-271 | H | OCH₃ | Br | H | H |
| A-272 | Br | OCHF₂ | H | H | H |
| A-273 | Br | H | OCHF₂ | H | H |
| A-274 | Br | H | H | OCHF₂ | H |
| A-275 | Br | H | H | H | OCHF₂ |
| A-276 | H | Br | OCHF₂ | H | H |
| A-277 | H | Br | H | OCHF₂ | H |
| A-278 | OCHF₂ | Br | H | H | H |
| A-279 | OCHF₂ | H | Br | H | H |
| A-280 | OCHF₂ | H | H | Br | H |
| A-281 | H | OCHF₂ | Br | H | H |
| A-282 | Br | OCF₃ | H | H | H |
| A-283 | Br | H | OCF₃ | H | H |
| A-284 | Br | H | H | OCF₃ | H |
| A-285 | Br | H | H | H | OCF₃ |
| A-286 | H | Br | OCF₃ | H | H |
| A-287 | H | Br | H | OCF₃ | H |
| A-288 | OCF₃ | Br | H | H | H |
| A-289 | OCF₃ | H | Br | H | H |
| A-290 | OCF₃ | H | H | Br | H |
| A-291 | H | OCF₃ | Br | H | H |
| A-292 | CH₃ | CHF₂ | H | H | H |
| A-293 | CH₃ | H | CHF₂ | H | H |
| A-294 | CH₃ | H | H | CHF₂ | H |
| A-295 | CH₃ | H | H | H | CHF₂ |
| A-296 | H | CH₃ | CHF₂ | H | H |
| A-297 | H | CH₃ | H | CHF₂ | H |
| A-298 | CHF₂ | CH₃ | H | H | H |
| A-299 | CHF₂ | H | CH₃ | H | H |
| A-300 | CHF₂ | H | H | CH₃ | H |
| A-301 | H | CHF₂ | CH₃ | H | H |
| A-302 | CH₃ | CF₃ | H | H | H |
| A-303 | CH₃ | H | CF₃ | H | H |
| A-304 | CH₃ | H | H | CF₃ | H |
| A-305 | CH₃ | H | H | H | CF₃ |
| A-306 | H | CH₃ | CF₃ | H | H |
| A-307 | H | CH₃ | H | CF₃ | H |
| A-308 | CF₃ | CH₃ | H | H | H |
| A-309 | CF₃ | H | CH₃ | H | H |
| A-310 | CF₃ | H | H | CH₃ | H |
| A-311 | H | CF₃ | CH₃ | H | H |
| A-312 | CH₃ | OCH₃ | H | H | H |
| A-313 | CH₃ | H | OCH₃ | H | H |
| A-314 | CH₃ | H | H | OCH₃ | H |
| A-315 | CH₃ | H | H | H | OCH₃ |
| A-316 | H | CH₃ | OCH₃ | H | H |
| A-317 | H | CH₃ | H | OCH₃ | H |
| A-318 | OCH₃ | CH₃ | H | H | H |
| A-319 | OCH₃ | H | CH₃ | H | H |
| A-320 | OCH₃ | H | H | CH₃ | H |
| A-321 | H | OCH₃ | CH₃ | H | H |
| A-322 | CH₃ | OCHF₂ | H | H | H |
| A-323 | CH₃ | H | OCHF₂ | H | H |
| A-324 | CH₃ | H | H | OCHF₂ | H |
| A-325 | CH₃ | H | H | H | OCHF₂ |
| A-326 | H | CH₃ | OCHF₂ | H | H |
| A-327 | H | CH₃ | H | OCHF₂ | H |
| A-328 | OCHF₂ | CH₃ | H | H | H |
| A-329 | OCHF₂ | H | CH₃ | H | H |
| A-330 | OCHF₂ | H | H | CH₃ | H |
| A-331 | H | OCHF₂ | CH₃ | H | H |
| A-332 | CH₃ | OCF₃ | H | H | H |
| A-333 | CH₃ | H | OCF₃ | H | H |
| A-334 | CH₃ | H | H | OCF₃ | H |
| A-335 | CH₃ | H | H | H | OCF₃ |
| A-336 | H | CH₃ | OCF₃ | H | H |
| A-337 | H | CH₃ | H | OCF₃ | H |
| A-338 | OCF₃ | CH₃ | H | H | H |
| A-339 | OCF₃ | H | CH₃ | H | H |
| A-340 | OCF₃ | H | H | CH₃ | H |
| A-341 | H | OCF₃ | CH₃ | H | H |
| A-342 | CHF₂ | CF₃ | H | H | H |
| A-343 | CHF₂ | H | CF₃ | H | H |
| A-344 | CHF₂ | H | H | CF₃ | H |
| A-345 | CHF₂ | H | H | H | CF₃ |
| A-346 | H | CHF₂ | CF₃ | H | H |
| A-347 | H | CHF₂ | H | CF₃ | H |
| A-348 | CF₃ | CHF₂ | H | H | H |
| A-349 | CF₃ | H | CHF₂ | H | H |
| A-350 | CF₃ | H | H | CHF₂ | H |
| A-351 | H | CF₃ | CHF₂ | H | H |
| A-352 | CHF₂ | OCH₃ | H | H | H |
| A-353 | CHF₂ | H | OCH₃ | H | H |
| A-354 | CHF₂ | H | H | OCH₃ | H |
| A-355 | CHF₂ | H | H | H | OCH₃ |
| A-356 | H | CHF₂ | OCH₃ | H | H |
| A-357 | H | CHF₂ | H | OCH₃ | H |
| A-358 | OCH₃ | CHF₂ | H | H | H |
| A-359 | OCH₃ | H | CHF₂ | H | H |
| A-360 | OCH₃ | H | H | CHF₂ | H |
| A-361 | H | OCH₃ | CHF₂ | H | H |
| A-362 | CHF₂ | OCHF₂ | H | H | H |
| A-363 | CHF₂ | H | OCHF₂ | H | H |
| A-364 | CHF₂ | H | H | OCHF₂ | H |
| A-365 | CHF₂ | H | H | H | OCHF₂ |
| A-366 | H | CHF₂ | OCHF₂ | H | H |
| A-367 | H | CHF₂ | H | OCHF₂ | H |
| A-368 | OCHF₂ | CHF₂ | H | H | H |
| A-369 | OCHF₂ | H | CHF₂ | H | H |
| A-370 | OCHF₂ | H | H | CHF₂ | H |
| A-371 | H | OCHF₂ | CHF₂ | H | H |
| A-372 | CHF₂ | OCF₃ | H | H | H |
| A-373 | CHF₂ | H | OCF₃ | H | H |
| A-374 | CHF₂ | H | H | OCF₃ | H |
| A-375 | CHF₂ | H | H | H | OCF₃ |
| A-376 | H | CHF₂ | OCF₃ | H | H |
| A-377 | H | CHF₂ | H | OCF₃ | H |
| A-378 | OCF₃ | CHF₂ | H | H | H |
| A-379 | OCF₃ | H | CHF₂ | H | H |
| A-380 | OCF₃ | H | H | CHF₂ | H |
| A-381 | H | OCF₃ | CHF₂ | H | H |
| A-382 | CF₃ | OCH₃ | H | H | H |
| A-383 | CF₃ | H | OCH₃ | H | H |
| A-384 | CF₃ | H | H | OCH₃ | H |
| A-385 | CF₃ | H | H | H | OCH₃ |
| A-386 | H | CF₃ | OCH₃ | H | H |
| A-387 | H | CF₃ | H | OCH₃ | H |
| A-388 | OCH₃ | CF₃ | H | H | H |
| A-389 | OCH₃ | H | CF₃ | H | H |
| A-390 | OCH₃ | H | H | CF₃ | H |
| A-391 | H | OCH₃ | CF₃ | H | H |
| A-392 | CF₃ | OCHF₂ | H | H | H |
| A-393 | CF₃ | H | OCHF₂ | H | H |
| A-394 | CF₃ | H | H | OCHF₂ | H |
| A-395 | CF₃ | H | H | H | OCHF₂ |
| A-396 | H | CF₃ | OCHF₂ | H | H |
| A-397 | H | CF₃ | H | OCHF₂ | H |
| A-398 | OCHF₂ | CF₃ | H | H | H |
| A-399 | OCHF₂ | H | CF₃ | H | H |
| A-400 | OCHF₂ | H | H | CF₃ | H |
| A-401 | H | OCHF₂ | CF₃ | H | H |
| A-402 | CF₃ | OCF₃ | H | H | H |
| A-403 | CF₃ | H | OCF₃ | H | H |
| A-404 | CF₃ | H | H | OCF₃ | H |
| A-405 | CF₃ | H | H | H | OCF₃ |
| A-406 | H | CF₃ | OCF₃ | H | H |
| A-407 | H | CF₃ | H | OCF₃ | H |
| A-408 | OCF₃ | CF₃ | H | H | H |
| A-409 | OCF₃ | H | CF₃ | H | H |
| A-410 | OCF₃ | H | H | CF₃ | H |
| A-411 | H | OCF₃ | CF₃ | H | H |
| A-412 | OCH₃ | OCHF₂ | H | H | H |
| A-413 | OCH₃ | H | OCHF₂ | H | H |
| A-414 | OCH₃ | H | H | OCHF₂ | H |
| A-415 | OCH₃ | H | H | H | OCHF₂ |
| A-416 | H | OCH₃ | OCHF₂ | H | H |
| A-417 | H | OCH₃ | H | OCHF₂ | H |
| A-418 | OCHF₂ | OCH₃ | H | H | H |
| A-419 | OCHF₂ | H | OCH₃ | H | H |
| A-420 | OCHF₂ | H | H | OCH₃ | H |
| A-421 | H | OCHF₂ | OCH₃ | H | H |
| A-422 | OCH₃ | OCF₃ | H | H | H |
| A-423 | OCH₃ | H | OCF₃ | H | H |
| A-424 | OCH₃ | H | H | OCF₃ | H |
| A-425 | OCH₃ | H | H | H | OCF₃ |
| A-426 | H | OCH₃ | OCF₃ | H | H |
| A-427 | H | OCH₃ | H | OCF₃ | H |
| A-428 | OCF₃ | OCH₃ | H | H | H |
| A-429 | OCF₃ | H | OCH₃ | H | H |
| A-430 | OCF₃ | H | H | OCH₃ | H |
| A-431 | H | OCF₃ | OCH₃ | H | H |
| A-432 | OCHF₂ | OCF₃ | H | H | H |
| A-433 | OCHF₂ | H | OCF₃ | H | H |
| A-434 | OCHF₂ | H | H | OCF₃ | H |
| A-435 | OCHF₂ | H | H | H | OCF₃ |
| A-436 | H | OCHF₂ | OCF₃ | H | H |
| A-437 | H | OCHF₂ | H | OCF₃ | H |
| A-438 | OCF₃ | OCHF₂ | H | H | H |
| A-439 | OCF₃ | H | OCHF₂ | H | H |
| A-440 | OCF₃ | H | H | OCHF₂ | H |
| A-441 | H | OCF₃ | OCHF₂ | H | H |
| A-442 | F | F | F | H | H |
| A-443 | F | F | H | F | H |
| A-444 | F | F | H | H | F |
| A-445 | F | H | F | F | H |
| A-446 | F | H | F | H | F |
| A-447 | H | F | F | F | H |
| A-448 | Cl | Cl | Cl | H | H |
| A-449 | Cl | Cl | H | Cl | H |
| A-450 | Cl | Cl | H | H | Cl |
| A-451 | Cl | H | Cl | Cl | H |
| A-452 | Cl | H | Cl | H | Cl |
| A-453 | H | Cl | Cl | Cl | H |
| A-454 | Br | Br | Br | H | H |
| A-455 | Br | Br | H | Br | H |
| A-456 | Br | Br | H | H | Br |
| A-457 | Br | H | Br | Br | H |
| A-458 | Br | H | Br | H | Br |
| A-459 | H | Br | Br | Br | H |
| A-460 | CH₃ | CH₃ | CH₃ | H | H |
| A-461 | CH₃ | CH₃ | H | CH₃ | H |
| A-462 | CH₃ | CH₃ | H | H | CH₃ |
| A-463 | CH₃ | H | CH₃ | CH₃ | H |
| A-464 | CH₃ | H | CH₃ | H | CH₃ |
| A-465 | H | CH₃ | CH₃ | CH₃ | H |
| A-466 | CF3 | CF3 | CF3 | H | H |
| A-467 | CF3 | CF3 | H | CF3 | H |
| A-468 | CF3 | CF3 | H | H | CF3 |
| A-469 | CF3 | H | CF3 | CF3 | H |
| A-470 | CF3 | H | CF3 | H | CF3 |
| A-471 | H | CF3 | CF3 | CF3 | H |
| A-472 | F | H | Cl | H | F |
| A-473 | F | H | F | H | Cl |
| A-474 | F | H | Cl | F | H |
| A-475 | F | H | Cl | H | Cl |
| A-476 | Cl | H | F | H | Cl |
| A-477 | Cl | H | Cl | F | H |
| A-478 | Cl | H | Cl | H | F |
| A-479 | Cl | H | F | F | H |
| A-480 | Cl | F | H | F | H |
| A-481 | F | H | H | F | Cl |
| A-482 | F | Cl | H | Cl | H |
| A-483 | F | Cl | H | H | Cl |
| A-484 | H | F | F | H | Cl |
| A-485 | Cl | F | H | H | F |
| A-486 | F | H | Cl | Cl | H |
| A-487 | F | H | CH3 | H | F |
| A-488 | F | H | F | H | CH3 |
| A-489 | F | H | CH3 | F | H |
| A-490 | F | H | CH3 | H | CH3 |
| A-491 | CH3 | H | F | H | CH3 |
| A-492 | CH3 | H | CH3 | F | H |
| A-493 | CH3 | H | CH3 | H | F |
| A-494 | CH3 | H | F | F | H |
| A-495 | CH3 | F | H | F | H |
| A-496 | F | H | H | F | CH3 |
| A-497 | F | CH3 | H | CH3 | H |
| A-498 | F | CH3 | H | H | CH3 |
| A-499 | H | F | F | H | CH3 |
| A-500 | CH3 | F | H | H | F |
| A-501 | F | H | CH3 | CH3 | H |
| A-502 | F | H | CF3 | H | F |
| A-503 | F | H | F | H | CF3 |
| A-504 | F | H | CF3 | F | H |
| A-505 | F | H | CF3 | H | CF3 |
| A-506 | CF3 | H | F | H | CF3 |
| A-507 | CF3 | H | CF3 | F | H |
| A-508 | CF3 | H | CF3 | H | F |
| A-509 | CF3 | H | F | F | H |
| A-510 | CF3 | F | H | F | H |
| A-511 | F | H | H | F | CF3 |
| A-512 | F | CF3 | H | CF3 | H |
| A-513 | F | CF3 | H | H | CF3 |
| A-514 | H | F | F | H | CF3 |
| A-515 | CF3 | F | H | H | F |
| A-516 | F | H | CF3 | CF3 | H |
| A-517 | Cl | H | CH3 | H | Cl |
| A-518 | Cl | H | Cl | H | CH3 |
| A-519 | Cl | H | CH3 | Cl | H |
| A-520 | Cl | H | CH3 | H | CH3 |
| A-521 | CH3 | H | Cl | H | CH3 |
| A-522 | CH3 | H | CH3 | Cl | H |
| A-523 | CH3 | H | CH3 | H | Cl |
| A-524 | CH3 | H | Cl | Cl | H |
| A-525 | CH3 | Cl | H | Cl | H |
| A-526 | Cl | H | H | Cl | CH3 |
| A-527 | Cl | CH3 | H | CH3 | H |
| A-528 | Cl | CH3 | H | H | CH3 |
| A-529 | H | Cl | Cl | H | CH3 |
| A-530 | CH3 | Cl | H | H | Cl |
| A-531 | Cl | H | CH3 | CH3 | H |
| A-532 | Cl | H | CF3 | H | Cl |
| A-533 | Cl | H | Cl | H | CF3 |
| A-534 | Cl | H | CF3 | Cl | H |
| A-535 | Cl | H | CF3 | H | CF3 |
| A-536 | CF3 | H | Cl | H | CF3 |
| A-537 | CF3 | H | CF3 | Cl | H |
| A-538 | CF3 | H | CF3 | H | Cl |
| A-539 | CF3 | H | Cl | Cl | H |
| A-540 | CF3 | Cl | H | Cl | H |
| A-541 | Cl | H | H | Cl | CF3 |
| A-542 | Cl | CF3 | H | CF3 | H |
| A-543 | Cl | CF3 | H | H | CF3 |
| A-544 | H | Cl | Cl | H | CF3 |
| A-545 | CF3 | Cl | H | H | Cl |
| A-546 | Cl | H | CF3 | CF3 | H |
| A-547 | CF3 | H | Cl | H | F |
| A-548 | Cl | H | CF3 | F | H |
| A-549 | CF3 | H | Cl | F | H |
| A-550 | CF3 | F | H | Cl | H |
| A-551 | Cl | H | H | F | CF3 |
| A-552 | Cl | H | CF3 | F | H |
| A-553 | Cl | H | CH3 | F | H |
| A-554 | CH3 | F | H | Cl | H |
| A-555 | CH3 | H | Cl | F | H |
| A-556 | Cl | H | H | F | CH3 |
| A-557 | Cl | H | CH3 | F | H |

Among the above compounds, preference is given to compounds of formulae I.1, I.2 and II.1, wherein R¹ is tert-butyl, and even more preference is given to compounds of formulae I.1, I.2 and II.1, wherein R¹ is tert-butyl and R⁴ or R^{4a} are hydrogen.

Compounds of formulae I and II can be prepared by one or more of the following methods and variations as described in schemes 1 to 7 and in the syntheses descriptions below. The variables are as defined above for formulae I and II.

Compounds of formula I, wherein R⁴ is H and m is 0 (or compounds II, wherein R^{4a} is H), can be prepared by sulfurizing the corresponding triazole derivative IV as outlined in scheme 1. Sulfurization can be carried out in analogy to known processes, for example as described in WO 96/16048. For instance, the triazolyl ring can be first deprotonated with a strong base, e.g. an organolithium base, such as n-butyllithium, tert-butyllithium or sec-butyllithium, lithium diisopropyl amide, sodium hydride, sodium amide or potassium tert-butylate mixed with tetramethylethylene diamine (TMEDA), and then the resulting anion is reacted with elemental sulfur. Sulfur is generally used in powdered form. The reaction is generally carried out in an inert solvent, such as ethers, e.g. diethylether, methyl-tert-butylether, tetrahydrofuran or dioxane, dimethoxyethane, liquid ammonia, dimethylsulfoxide or dimethylformamide. The reaction temperature is not very critical and can range, for example, from −70 to +50° C., preferably from −70 to 0° C. Alternatively, sulfurization can be carried out in the absence of a base by reacting 7 with elemental sulfur in a high-boiling solvent, such as N-methylpyrrolidinone, dioxane or N,N-dimethylformamide, while heating, e.g. to 160 to 250° C. After completion of the reaction, the resulting mixture is hydrolyzed, e.g. by the addition of water or an aqueous acid, such as a mineral acid (e.g. dilute sulfuric acid or hydrochloric acid), acetic acid or ammoniumchloride, to give compound I.

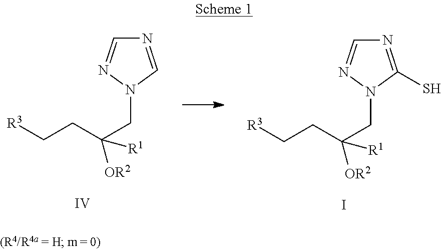

The triazole compound IV, wherein R² is H, can be prepared in analogy to known methods, such as described, for example, in DE-A-3702301, EP-A-40345 or EP-A-52424, as outlined in scheme 2. For instance, the oxirane compound I and [1,2,4]-1H-triazole can be reacted in the presence of a base, such as an alkali metal hydride (e.g. sodium hydride, potassium hydride), an alkali metal hydroxide (e.g. sodium hydroxide, potassium hydroxide), or an alkali metal carbonate (e.g. sodium carbonate, potassium carbonate, caesium carbonate). The reaction is suitably carried out in a solvent. Suitable solvents are, for example, toluene, N-methypyrrolidinone, ethers (e.g. diethyl ether, tetrahydrofuran), alcohols (e.g. methanol, ethanol, isopropanol or tert-butanol), acetonitrile, or N,N-dimethylformamide.

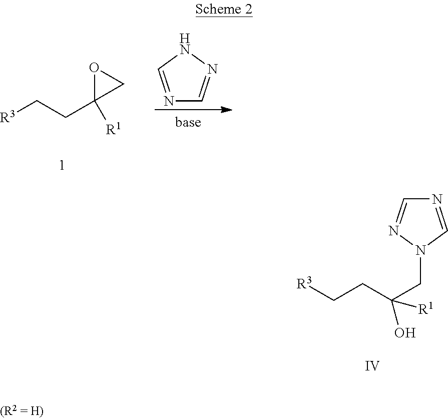

The oxirane 1 in turn can be prepared in analogy to known methods, such as described, for example, in EP-A-0267778, EP-A-40345, EP-A-52424, Org. Syn. 49, 78 (1968) or J. Am. Chem. Soc. 1975, 1353, as outlined in scheme 3 below. For instance, the ketone 2 may be reacted with a sulfonium ylide or an oxosulfonium ylide, such as dimethyloxosulfonium methylide or dimethylsulfonium methylide in a solvent. Alternatively, the oxirane 1 can be prepared in an epoxidation reaction in analogy to the method described in Tetrahedron Lett. 23, 5283 (1982 or in EP-A-0655443 by subjecting 2 to the reaction with a trimethylsulfonium salt, such as trimethylsulfonium bromide, trimethylsulfonium iodide or methyltrimethylsulfonium sulfate, in the presence of a metal oxide, such as alkaline metal oxides (e.g. sodium oxide, potassium oxide), alkaline earth metal oxides (e.g. magnesium oxide, calcium oxide, barium oxide) or zinc oxide, and optionally a base, such as alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate) in a two-phase solid/liquid system comprising an organic solvent, such as toluene, N-methypyrrolidinone, ethers (e.g. diethyl ether, tetrahydrofuran), acetonitrile or N,N-dimethylformamide. Alternatively, the oxirane 1 can be prepared in analogy to the method described in Tetrahedron 1985, 1259 by epoxidation of 2 with a trimethylsulfonium salt, such as trimethylsulfonium bromide, trimethylsulfonium iodide or methyl-trimethylsulfonium sulfate, or a trimethylsulfoxonium salt, such as trimethylsulfoxonium bromide, trimethylsulfoxonium iodide or methyltrimethylsulfoxonium sulfate and potassium sulfate/aluminium oxide.

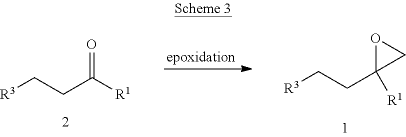

The ketone 2 can be obtained from the halide 4 by a Grignard reaction with the aldehyde 5, as outlined in scheme 4 below. Oxidation of the obtained alcohol 3 via known methods, such as oxidation with the Swern reagent, hypervalent iodine compounds (IBX, Martin's reagent), chromine compounds (e.g. pyridinium dichromate, pyridinium chlorochromate, dipyridinium chromine trioxide), sodium hypochlorite and the like, yields the ketone 2.

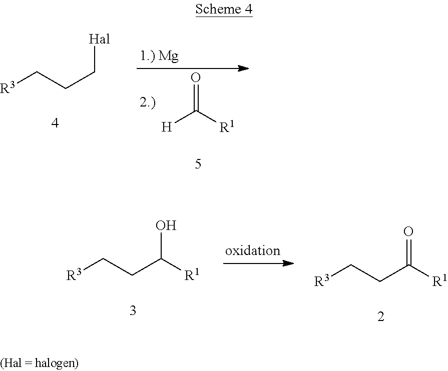

As an alternative to the process described in scheme 3, the oxirane 1 can be prepared in analogy to the method described in Org. Syn. 40, 66, 1966, J. Org. Chem. 28, 1128, 1963 and Org. Syn. Coll. Vol. 4, 552, 1963 as outlined in scheme 5 below by first subjecting the ketone 2 to a Wittig reaction, thus yielding the corresponding olefinic compound 6, and then subjecting this to an epoxidation reaction. The Wittig reaction can be carried out under standard conditions, such as the use of methyltriphenylphosphonium bromide or iodide in the presence of an alkali metal base, such as n-butyllithium, sec-butyllithium or tert-butyllithium. Epoxidation can also be carried out with standard reagents, such as peracetic acid, perbenzoic acid meta-chloroperbenzoic acid, perphthalic acid and the like. Olefination (i.e. transformation of the C═O into a C═CH₂ group) of 5 can alternatively be achieved by the use of Tebbe's reagent ((C₅H₅)₂TiCH₂ClAl(CH₃)₂).

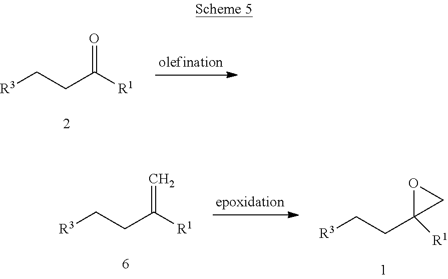

As an alternative to the process described in schemes 2 and 3, the oxirane 1 and the triazole IV can be prepared in analogy to the methods described in U.S. Pat. No. 4,243,405, U.S. Pat. No. 4,929,735, U.S. Pat. No. 5,691,363 and WO 97/43269, by subjecting the aldehyde 7 and the ketone 8 to an aldol condensation to give the enone 9. This can either be first hydrogenated to the ketone 2, which is the epoxidized as described above to the oxirane 1, or it can be first epoxidized to the oxirane 10, then ring-opened with [1,2,4]-triazole to 11 and finally hydrogenated to IV, as shown in scheme 6 below.

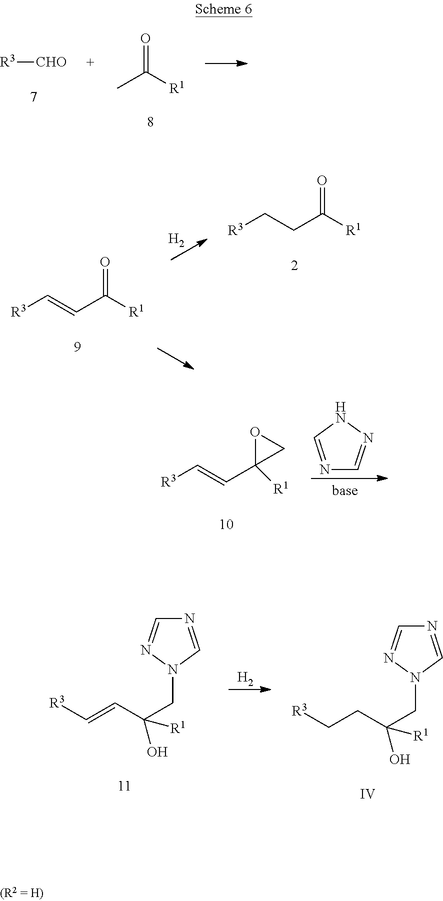

As an alternative to the process described in scheme 1, compounds I, wherein R⁴ is H and m is 0 (or compounds II, wherein R^{4a} is H), can also be prepared in analogy to the method described in WO 99/18088 as outlined in scheme 7 below. Epoxide opening of 1 with hydrazine, optionally in the presence of an acid (e.g. hydrochloric acid, hydrobromic acid, acetic acid, sulfuric acid or p-toluenesulfonic acid) or a base (e.g. triethyl-amine, diisopropylethylamine, sodium carbonate or potassium carbonate) in a suitable solvent, such as an alcohol (e.g. methanol, ethanol, isopropanol, tert-butanol), N-methylpyrrolidinone, an ether (e.g. diethyl ether, tetrahydrofuran, dioxane, 1,2-dimethoxyethane), acetonitrile, N,N-dimethylformamide or dimethylsulfoxide, yields 12. This is then converted into the semicarbazide 13 by reaction with a thiocyanate, such as sodium thiocyanate, potassium thiocyanate or ammonium thiocyanate (i.e. M⁺=e.g. Na⁺, K⁺, NH₄⁺), in a suitable solvent, such as an alcohol (e.g. methanol, ethanol, iso-propanol, tert-butanol), N-methylpyrrolidinone, an ether (e.g. diethyl ether, tetrahydrofuran, dioxane, 1,2-dimethoxyethane), acetonitrile, N,N-dimethylformamide, dimethylsulfoxide, toluene or xylene. The semicarbazide is then converted into I/II via reaction with a formic acid alkyl ester (e.g. formic acid methyl ester, formic acid ethyl ester) in a solvent. Suitable solvents are, for example, alcohols (e.g. methanol, ethanol, isopropanol, tert-butanol), N-methylpyrrolidinone, ethers (e.g. diethyl ether, tetrahydrofuran, dioxane, 1,2-dimethoxyethane), acetonitrile, N,N-dimethylformamide, dimethylsulfoxide, toluene or xylene. Alternatively, 12 can be reacted with hydrogen thiocyanate and formaldehyde in a solvent. Suitable solvents are, for example, alcohols (e.g. methanol, ethanol, isopropanol, tert-butanol), N-methylpyrrolidinone, ethers (e.g. diethyl ether, tetrahydrofuran, dioxane, 1,2-dimethoxyethane), acetonitrile, N,N-dimethylformamide, dimethylsulfoxide, toluene or xylene. The resulting triazolidinthione 14 is then oxidized using, for example, FeCl₃ in an aqueous acid (e.g. hydrochloric acid) or oxygen in the presence of an alkali metal hydroxide (e.g. sodium hydroxide, potassium hydroxide) and elemental sulfur to I/II. In a yet further alternative, 12 is reacted with a dialkyl ketone (e.g. acetone, diethylketone, methyl ethyl ketone) and a thiocyanate (e.g. sodium thiocyanate, potassium thiocyanate, ammonium thiocyanate) in a solvent to give the triazolidinthione 15. Suitable solvents are, for example, alcohols (e.g. methanol, ethanol, isopropanol, tert-butanol), N-methylpyrrolidinone, ethers (e.g. diethyl ether, tetra-hydrofuran, dioxane, 1,2-dimethoxyethane), acetonitrile, N,N-dimethylformamide, di-methylsulfoxide, toluene or xylene. The triazolidinthione 15 is then converted into I/II by reaction with formic acid in the presence of an acid (e.g. hydrochloric acid, hydrobromic acid, acetic acid, sulfuric acid, p-toluenesulfonic acid) or a metal oxide (e.g. amorphous TiO₂).

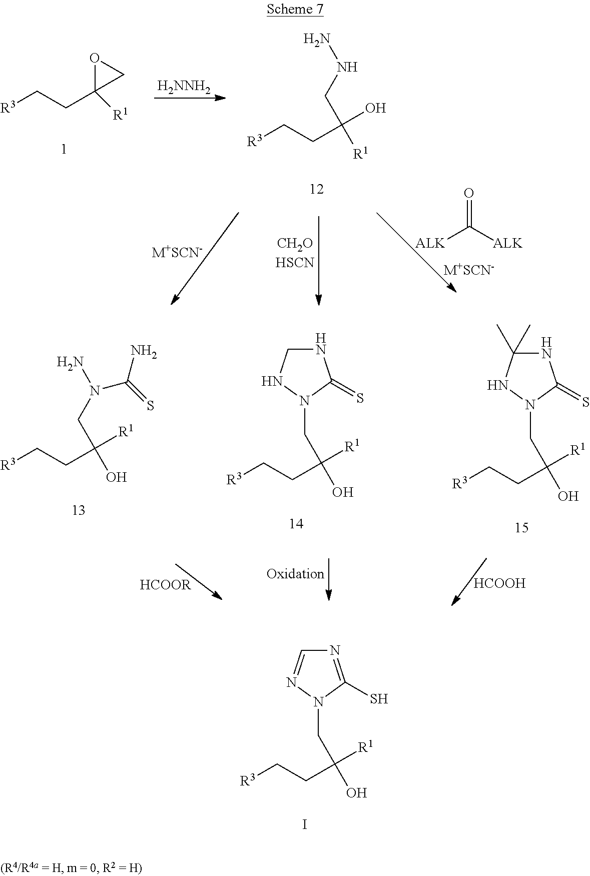

The halide 4, the aldehydes 5 and 7 and the ketone 8 used in the above reactions are either commercially available or can be produced by standard methods known to the skilled person.

Compounds of formula I, wherein R⁴ is different from hydrogen and m is 0, can be prepared from compounds I (wherein R⁴═H and m=0).

Compounds of formula I, wherein m is 0 and R⁴ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as described above, and a 5- or 6-membered saturated, partially unsaturated or aromatic heterocyclic ring containing 1, 2 or 3 heteroatoms selected from N, O and S as ring members, where the heterocyclic ring may be substituted as described above, may be prepared in analogy to the method described in DE-A-19520098 by reacting a compound I, wherein m is 0 and R⁴ is H, with a compound R⁴-LG, where R⁴ has one of the above meanings and LG is a leaving group, such as a halide (e.g. Cl, Br, I), a tosylate or a mesylate, in the presence of a base. Suitable bases are, for example, alkali metal hydrides (e.g. sodium hydride, potassium hydride), alkali metal hydroxides (e.g. sodium hydroxide, potassium hydroxyide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium and lithium diisopropylamine.). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Alternatively, compounds of formula I, wherein m is 0 and R⁴ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as described above, and a 5- or 6-membered saturated, partially unsaturated or aromatic heterocyclic ring containing 1, 2 or 3 heteroatoms selected from N, O and S as ring members, where the heterocyclic ring may be substituted as described above, may be prepared in analogy to the method described in Heterocycles, 23(7), 1645-1649, 1985 by reacting compound IV with a disulfide R⁴—S—S—R⁴ in the presence of a strong base under conditions similar to those described for scheme 1.

Compounds of formulae I, wherein m is 0 and R⁴ is —C(═O)R¹⁰ or —C(═S)R¹⁰, may be prepared in analogy to the method described in DE-A-19617461 by reacting a compound I, wherein m is 0 and R⁴ is H, with a compound R¹⁰—C(═O)—W, R¹⁰—C(═S)—W, R^{10′}—N═C═O or R^{10′}—N═C═S, wherein R¹⁰ has one of the above meanings, R^{10′} _{is C}₁-C₁₀-alkyl or C₁-C₁₀-haloalkyl and W is a good leaving group, such as a halide (e.g. Cl, Br, I), an alkoxide (e.g. methoxide, ethoxide) or pentafluorophenoxide, in the presence of a base. Suitable bases are, for example, alkali metal hydrides (e.g. sodium hydride, potassium hydride), alkali metal hydroxides (e.g. sodium hydroxide, potassium hydroxyide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁴ is —SO₂R¹⁰, may be prepared in analogy to the method described in DE-A-19620590 by reacting a compound I, wherein m is 0 and R⁴ is H, with a compound R¹⁰—SO₂—W, wherein R¹⁰ has one of the above meanings and W is a good leaving group, such as a halide (e.g. Cl, Br, I), an alkoxide (e.g. methoxide, ethoxide) or pentafluorophenoxide, in the presence of a base. Suitable bases are, for example, alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁴ is —CN, may be prepared in analogy to the method described in DE-A-19620407 by reacting a compound I, wherein m is 0 and R⁴ is H, with a compound CN—W, wherein W is a good leaving group, such as a halide (e.g. Cl, Br, I), in the presence of a base. Suitable bases are, for example, alkali metal hydrides (e.g. sodium hydride, potassium hydride), alkali metal hydroxides (e.g. sodium hydroxide, potassium hydroxide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁴ is M, may be prepared in analogy to the method described in DE-A-19617282 by reacting a compound I with an amine NR^{a}R^{b}R^{c}, wherein R^{a}, R^{b} and R^{c} are as defined above, or with a metal salt, such as sodium hydroxide, potassium hydroxide or copper acetate.

Compounds of formula I, wherein m is 0 and R⁴ is a group of formula III, may be prepared in analogy to the method described in WO 97/43269 by reacting a compound I, wherein m is 0 and R⁴ is H, with a halogen, especially iodine, in the presence of a base. Suitable bases are, for example, alkali metal hydrides (e.g. sodium hydride, potassium hydride), alkali metal hydroxides (e.g. sodium hydroxide, potassium hydroxyide), alkali metal carbonates (e.g. sodium carbonate, potassium carbonate, caesium carbonate), alkali metal alkoxides (e.g. sodium methoxide, potassium methoxide, sodium ethoxide, potassium ethoxide, potassium tert-butoxide) or organolithium bases (e.g. n-butyl lithium, sec-butyl lithium, tert-butyl lithium, lithium diisopropylamine). The reaction is generally carried out in a suitable solvent. Suitable solvents are, for example, toluene, N-methylpyrrolidinone, ethers (e.g. diethyl ether, tetrahydrofuran, dioxane, 1,2-dimethoxyethane), acetonitrile, N,N-dimethylformamide or dimethylsulfoxide.

Compounds of formula I, wherein m is 0 and R⁴ is —P(=Q)R¹¹R¹², may be prepared in analogy to the method described in WO 99/05149.

Compounds of formula II, wherein R^{4a} is hydrogen (or compounds of formula I, wherein m is 0 and R⁴ is hydrogen), can be prepared in analogy to the method described in WO 99/18087 by reacting a triazolidinthione 14 with an oxidizing agent, optionally in the presence of a catalyst. Suitable oxidizing agents are, for example, oxygen, sulfur and potassium superoxide. Especially in case oxygen is used as oxidizing agent, it is advantageous to carry out the oxidation reaction in the presence of a catalyst. A suitable catalyst is, for example, a mixture of powdery sulfur and KOH. The reaction is generally carried out in a suitable solvent. Suitable solvents are, for example, aliphatic hydrocarbons (e.g. pentane, hexane), cycloaliphatic hydrocarbons (e.g. cyclohexane), aromatic hydrocarbons (e.g. bemzene, toluene, the xylenes), ethers (e.g. diethylether, methyl-tert-butylether), and esters (e.g. ethylecetate, propylacetate, n-butylacetate).

The oxidation of the triazolidinthione 14 may also be carried out with ferric chloride (FeCl₃) in an acidic aqueous solution in analogy to the method described in WO 01/46158. The reaction is generally carried out in a suitable solvent. Suitable solvents are, for example, ethanol, ethylacetate and mixtures of ethanol with toluene.

The oxidation of the triazolidinthione 14 may also be carried out with formic acid, optionally in the presence of a catalyst, in analogy to the method described in WO 99/18086 or WO 99/18088. Suitable catalysts are, for example, acids, like hydrochloric acid, sulfuric acid or p-toluenesulfonic acid, and metal oxides, like amorphous titanium dioxide. The reaction is generally carried out in a suitable solvent. Suitable solvents are weakly polar solvents like, for example, alcohols such as propanol, butanol and pentanol, esters, like ethyl acetate, butyl acetate and isobutyl formate, ethers, like 1,2-dimethoxyethane, methyl-tert-butyl ether and methyl-tert-amylether, and formic acid used in excess.

Compounds of formula II, wherein R^{4a} is different from hydrogen, can be prepared by reacting the NR^{4a} group, wherein R^{4a} is H, in analogy to the above-described conversion of compounds I, wherein R⁴ is H, into compounds, wherein R⁴ is different from H.

Compounds I, wherein m is 1 or 2, can be prepared from respective compounds I, wherein m is 0, by oxidation. Alternatively, compounds I, wherein m is 2, can be prepared from compounds IV by first deprotonating the triazolyl ring and then reacting with a sulfonyl chloride R⁴SO₂Cl. Compounds I, wherein m is 3, can be prepared from compounds IV by first deprotonating the triazolyl ring and then reacting with sulfuric acid chloride or a sulfuric ester chloride of formula R⁴⁰SO₂Cl, wherein R⁴ is selected from hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, where the phenyl moiety in the 2 last-mentioned radicals may be substituted as mentioned above, and a 5- or 6-membered saturated, partially unsaturated or aromatic heterocyclic ring containing 1, 2 or 3 heteroatoms selected from N, O and S, wherein the heterocyclic ring may be substituted as mentioned above.

Compounds of formula I or II, wherein R² is different from hydrogen, can be prepared by introducing a protective group R² by known means in any reaction step in which the protection of the OH group is required or advantageous. Deprotection to R²═H can be carried out by any known means.

If individual compounds cannot be prepared via the above-described routes, they can be prepared by derivatization of other compounds I and II or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

A further aspect of the invention relates to compounds of formula IV

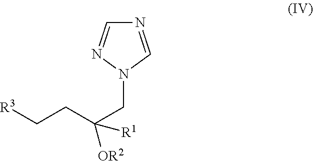

wherein R¹, R² and R³ have one of the general or, in particular, one of the preferred meanings given above for compounds I and II, with the proviso that R⁷ is not 4-F, 4-Cl, 2,4-Cl₂,3,4-Cl₂,2-CH₃,4-CH₃,4-C(CH₃)₃,4-CF₃,4-OCF₃,4-phenyl which may carry 1, 2 or 3 substituents R⁵ or 4-phenoxy which may carry 1, 2 or 3 substituents R⁵ if R¹ is isopropyl, tert-butyl, 1-methylcyclopropyl, 1-chlorocyclopropyl, cyclohexyl or 4-(4-chlorophenyl), and preferably with the proviso that R⁷ is not F, Cl, C₁-C₄-alkyl, C₁-C₄-fluoroalkyl, C₁-C₄-fluoroalkoxy, phenyl which may carry 1, 2 or 3 substituents R⁵ or 4-phenoxy which may carry 1, 2 or 3 substituents R⁵, if R¹ is isopropyl, tert-butyl, 1-methylcyclopropyl, 1-chlorocyclopropyl, cyclohexyl or 4-(4-chlorophenyl).

Compounds IV are on the one side valuable intermediates in the preparation of compounds I and II (see above schemes), but on the other side show a remarkable fungicidal activity, too.

Particularly preferred compounds IV are compounds of formulae IV.1, wherein the combination of R⁷¹, R⁷², R⁷³, R⁷⁴ and R⁷⁵ corresponds in each case to one row in table A above and R¹ is tert-butyl, cyclopropyl, 1-methylcyclopropyl, 1-chlorocyclopropyl, cyclopropylmethyl or 1-cyclopropylethyl.

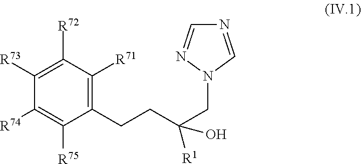

A further aspect of the invention relates to compounds of formula 7

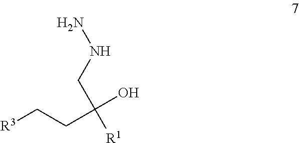

wherein R¹ and R³ have one of the general or, in particular, one of the preferred meanings given above for compounds I and II.

Compounds 7 are valuable intermediates in the preparation of compounds I and II (see above schemes).

A further aspect of the invention relates to compounds of formula 1

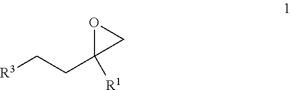

wherein R¹ and R³ have one of the general or, in particular, one of the preferred meanings given above for compounds I and II.

Compounds I are valuable intermediates in the preparation of compounds I and II (see above schemes).

The invention further refers to an agricultural composition comprising at least one compound of formula I, II and/or IV as defined above or an agriculturally acceptable salt thereof and a liquid or solid carrier. Suitable carriers, as well as auxiliaries and further active compounds which may also be contained in the composition of the invention are defined below.

The compounds I and II as well as IV and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I, II and IV and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, e.g. conifers; and on the plant propagation material, such as seeds, and the crop material of these plants.

Preferably, compounds I, II and IV and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I, II and IV and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

Plants that have been modified by breeding, mutagenesis or genetic engineering, e.g. have been rendered tolerant to applications of specific classes of herbicides, such as hydroxyphenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/026390, WO 97/41218, WO 98/002526, WO 98/02527, WO 04/106529, WO 05/20673, WO 03/014357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A 242 236, EP-A 242 246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox. Genetic engineering methods have been used to render cultivated plants, such as soybean, cotton, corn, beets and rape, tolerant to herbicides such as glyphosate and glufosinate, some of which are commercially available under the trade names RoundupReady® (glyphosate-tolerant, Monsanto, U.S.A.) and LibertyLink® (glufosinate-tolerant, Bayer CropScience, Germany).

Furthermore, plants are also covered that, by the use of recombinant DNA techniques, are capable to synthesize one or more insecticidal proteins, especially those known from the bacterial genus Bacillus, particularly from Bacillus thuringiensis, such as δ-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g., in the publications mentioned above. These insecticidal proteins contained in the genetically modified plants impart to the plants producing these proteins tolerance to harmful pests from all taxonomic groups of arthropods, especially to beetles (Coleoptera), two-winged insects (Diptera), and moths (Lepidoptera) and to nematodes (Nematoda). Genetically modified plants capable to synthesize one or more insecticidal proteins are, e.g., described in the publications mentioned above, and some of them are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e.g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that, by the use of recombinant DNA techniques, are capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e.g. potato cultivars, which express resistance genes acting against Phytophthora infestans derived from the Mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g., in the publications mentioned above.

Furthermore, plants are also covered that, by the use of recombinant DNA techniques, are capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that, by the use of recombinant DNA techniques, contain a modified amount of substances of content or new substances of content, specifically to improve human or animal nutrition, e.g. oil crops that produce health-promoting long-chain omega-3 fatty acids or unsaturated omega-9 fatty acids (e.g. Nexera® rape, DOW Agro Sciences, Canada).

Furthermore, plants are also covered that, by the use of recombinant DNA techniques, contain a modified amount of substances of content or new substances of content, specifically to improve raw material production, e.g. potatoes that produce increased amounts of amylopectin (e.g. Amflora® potato, BASF SE, Germany).

The compounds I, II and IV and compositions thereof, respectively, are particularly suitable for controlling the following plant diseases: Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternate), tomatoes (e.g. A. solani or A. alternate) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchil) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtuse; Elsinoe spp. on pome fruits (E. pyn), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani on soybeans and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grain-staining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsid), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or, rotbrenner', anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, and asparagus (e.g. P. asparagi); Pyrenophora (anamorph: Drechslera) triticirepentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

The compounds I, II and IV and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, coiling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The compounds I, II and IV and compositions thereof, respectively, may be used for improving the health of a plant. The invention also relates to a method for improving plant health by treating a plant, its propagation material and/or the locus where the plant is growing or is to grow with an effective amount of compounds I, II and/or IV and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor [e.g. improved plant growth and/or greener leaves (“greening effect”)], quality (e.g. improved content or composition of certain ingredients) and tolerance to abiotic and/or biotic stress. The above identified indicators for the health condition of a plant may be interdependent or may result from each other.

The compounds of formula I, II and IV can be present in different crystal modifications whose biological activity may differ. They are likewise subject matter of the present invention.

The compounds I, II and IV are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I, II and/or IV as such or a composition comprising at least one compound I, II and/or IV prophylactically either at or before planting or transplanting.

The invention also relates to agrochemical compositions comprising a solvent or solid carrier and at least one compound I, II and/or IV and to the use for controlling harmful fungi.

An agrochemical composition comprises a fungicidally effective amount of a compound I, II and/or IV. The term “effective amount” denotes an amount of the composition or of the compounds I, II and/or IV, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, II and IV and salts thereof can be converted into customary types of agrochemical compositions, e.g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e.g. SC, OD, FS, EC, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, pp. 8-57 et seq., WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and inorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalene-sulfonic acid (Nekal® types, BASF, Germany), and fatty acids, alkylsulfonates, alkyl-arylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearyl-phenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinyl-amines (Lupasol® types, BASF, Germany), polyvinylpyrrolidone and the copolymers thereof.

Examples for thickeners (i.e. compounds that impart a modified flowability to compositions, i.e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Bactericides may be added for preservation and stabilization of the composition. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned and the designations rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Examples for composition types are:

1. Composition Types for Dilution with Water

### i) Water-Soluble Concentrates (SL, LS)

10 parts by weight of a compound I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

### ii) Dispersible Concentrates (DC)

20 parts by weight of a compound I according to the invention are dissolved in 70 parts by weight of cyclohexanone with addition of 10 parts by weight of a dispersant, e.g. polyvinylpyrrolidone. Dilution with water gives a dispersion. The active substance content is 20% by weight.

iii) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

### iv) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

### v) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

### vi) Water-Dispersible Granules and Water-Soluble Granules (WG, SG)

50 parts by weight of a compound I according to the invention are ground finely with addition of 50 parts by weight of dispersants and wetting agents and prepared as water-dispersible or water-soluble granules by means of technical appliances (e.g. extrusion, spray tower, fluidized bed). Dilution with water gives a stable dispersion or solution of the active substance. The composition has an active substance content of 50% by weight.

vii) Water-Dispersible Powders and Water-Soluble Powders (WP, SP, SS, WS)

75 parts by weight of a compound I according to the invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

### 2. Composition Types to be Applied Undiluted

### ix) Dustable Powders (DP, DS)

5 parts by weight of a compound I according to the invention are ground finely and mixed intimately with 95 parts by weight of finely divided kaolin. This gives a dustable composition having an active substance content of 5% by weight.

### x) Granules (GR, FG, GG, MG)

0.5 parts by weight of a compound I according to the invention is ground finely and associated with 99.5 parts by weight of carriers. Current methods are extrusion, spray-drying or the fluidized bed. This gives granules to be applied undiluted having an active substance content of 0.5% by weight.

### xi) ULV Solutions (UL)

10 parts by weight of a compound I according to the invention are dissolved in 90 parts by weight of an organic solvent, e.g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typically, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are, e.g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

The compositions according to the invention can, in the use form as fungicides, also be present together with other active substances, e.g. with herbicides, insecticides, growth regulators, fungicides or else with fertilizers, as pre-mix or, if appropriate, not until immediately prior to use (tank mix).

Mixing the compounds I, II and/or IV or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:

### A) Strobilurins

azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenyl)-acrylic acid methyl ester, methyl (2-chloro-5-[1-(3-methylbenzyloxyimino)ethyl]benzyl)carbamate and 2-(2-(3-(2,6-dichlorophenyl)-1-methyl-allylideneaminooxymethyl)-phenyl)-2-methoxyimino-N-methyl-acetamide;
   B) carboxamides
carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methyl-thiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide and N-(2-(1,3,3-trimethyl-butyl)-phenyl)-1,3-dimethyl-5-fluoro-1H-pyrazole-4-carboxamide;
carboxylic morpholides: dimethomorph, flumorph, pyrimorph;
benzoic acid amides: flumetover, fluopicolide, fluopyram, zoxamide, N-(3-Ethyl-3,5,5-trimethyl-cyclohexyl)-3-formylamino-2-hydroxy-benzamide;
other carboxamides: carpropamid, dicyclomet, mandiproamid, oxytetracyclin, silthiofarm and N-(6-methoxy-pyridin-3-yl)cyclopropanecarboxylic acid amide;

### C) Azoles

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;
imidazoles: cyazofamid, imazalil, pefurazoate, prochloraz, triflumizol;
benzimidazoles: benomyl, carbendazim, fuberidazole, thiabendazole;
others: ethaboxam, etridiazole, hymexazole and 2-(4-chloro-phenyl)-N-[4-(3,4-di-methoxy-phenyl)-isoxazol-5-yl]-2-prop-2-ynyloxy-acetamide;

### D) Heterocyclic Compounds

pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fenpiclonil, fludioxonil;
morpholines: aldimorph, dodemorph, dodemorph-acetate, fenpropimorph, tridemorph;
piperidines: fenpropidin;
dicarboximides: fluoroimid, iprodione, procymidone, vinclozolin;
non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;
others: acibenzolar-S-methyl, amisulbrom, anilazin, blasticidin-S, captafol, captan, chinomethionat, dazomet, debacarb, diclomezine, difenzoquat, difenzoquatmethylsulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine and 5-ethyl-6-octyl-[1,2,4]triazolo[1,5-a]pyrimidine-7-ylamine;

### E) Carbamates

thio- and dithiocarbamates: ferbam, mancozeb, maneb, metam, methasulphocarb, metiram, propineb, thiram, zineb, ziram;
carbamates: benthiavalicarb, diethofencarb, iprovalicarb, propamocarb, propamocarb hydrochlorid, valiphenal and N-(1-(1-(4-cyano-phenyl)ethanesulfonyl)-but-2-yl) carbamic acid-(4-fluorophenyl) ester;

### F) Other Active Substances

guanidines: guanidine, dodine, dodine free base, guazatine, guazatine-acetate, iminoctadine, iminoctadine-triacetate, iminoctadine-tris(albesilate);
antibiotics: kasugamycin, kasugamycin hydrochloride-hydrate, streptomycin, polyoxine, validamycin A;
nitrophenyl derivates: binapacryl, dinobuton, dinocap, nitrthal-isopropyl, tecnazen,
organometal compounds: fentin salts, such as fentin-acetate, fentin chloride or fentin hydroxide;
sulfur-containing heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;
inorganic active substances: Bordeaux mixture, copper acetate, copper hydroxyide, copper oxychloride, basic copper sulfate, sulfur;
others: biphenyl, bronopol, cyflufenamid, cymoxanil, diphenylamin, metrafenone, mildiomycin, oxin-copper, prohexadione-calcium, spiroxamine, tolylfluanid, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl form amidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, acetic acid 6-tert-butyl-8-fluoro-2,3-dimethyl-quinolin-4-yl ester and methoxy-acetic acid 6-tert-butyl-8-fluoro-2,3-dimethyl-quinolin-4-yl ester.

### G) Growth Regulators

abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

### H) Herbicides

acetamides: acetochlor, alachlor, butachlor, dimethachlor, dimethenamid, flufenacet, mefenacet, metolachlor, metazachlor, napropamide, naproanilide, pethoxamid, pretilachlor, propachlor, thenylchlor;
amino acid derivatives: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
Bipyridyls: diquat, paraquat;
(thio)carbamates: asulam, butylate, carbetamide, desmedipham, dimepiperate, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bomoxynil, dichlobenil, ioxynil;
imidazolinones: imazamethabenz, imazamox, imazapic, imazapyr, imazaquin, imazethapyr;
phenoxy acetic acids: clomeprop, 2,4-dichlorophenoxyacetic acid (2,4-D), 2,4-DB, dichlorprop, MCPA, MCPA-thioethyl, MCPB, Mecoprop;
pyrazines: chloridazon, flufenpyr-ethyl, fluthiacet, norflurazon, pyridate;
pyridines: aminopyralid, clopyralid, diflufenican, dithiopyr, fluridone, fluoroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-Asulfonyl)-3-(4,6-dimethoxy-pyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, methabenzthiazuron, tebuthiuron;
other acetolactate synthase inhibitors: bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

### I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemetonmethyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, taufluvalinate, tefluthrin, tetramethrin, tralomethrin, transfluthrin, profluthrin, dimefluthrin;
insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
nicotinic receptor agonists/antagonists compounds: clothianidin, dinotefuran, imidacloprid, thiamethoxam, nitenpyram, acetamiprid, thiacloprid, 1-(2-chloro-thiazol-5-ylmethyl)-2-nitrimino-3,5-dimethyl-[1,3,5]triazinane;
GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
macrocyclic lactone insecticides: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III compounds: acequinocyl, fluacyprim, hydramethylnon;
Uncouplers: chlorfenapyr;
oxidative phosphorylation inhibitors: cyhexatin, diafenthiuron, fenbutatin oxide, propargite;
moulting disruptor compounds: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluoron, and pyrifluquinazon.

The present invention furthermore relates to agrochemical compositions comprising a mixture of at least one compound I, II and/or IV (component 1) and at least one further active substance useful for plant protection, e.g. selected from the groups A) to I) (component 2), in particular one further fungicide, e.g. one or more fungicide from the groups A) to F), as described above, and if desired one suitable solvent or solid carrier. Those mixtures are of particular interest, since many of them at the same application rate show higher efficiencies against harmful fungi. Furthermore, combating harmful fungi with a mixture of compounds I, II and/or IV and at least one fungicide from groups A) to F), as described above, is more efficient than combating those fungi with individual compounds I, II or IV or individual fungicides from groups A) to F). By applying compounds I, II and/or IV together with at least one active substance from groups A) to I) a synergistic effect can be obtained, i.e. more then simple addition of the individual effects is obtained (synergistic mixtures).

According to this invention, applying the compounds I, II and/or IV together with at least one further active substance is to be understood to denote that at least one compound of formula I, II and/or IV and at least one further active substance occur simultaneously at the site of action (i.e. the harmful fungi to be controlled or their habitats such as infected plants, plant propagation materials, particularly seeds, surfaces, materials or the soil as well as plants, plant propagation materials, particularly seeds, soil, surfaces, materials or rooms to be protected from fungal attack) in a fungicidally effective amount. This can be obtained by applying the compounds I, II and/or IV and at least one further active substance simultaneously, either jointly (e.g. as tank-mix) or sperately, or in succession, wherein the time interval between the individual applications is selected to ensure that the active substance applied first still occurs at the site of action in a sufficient amount at the time of application of the further active substance(s). The order of application is not essential for working of the present invention.

In binary mixtures, i.e. compositions according to the invention comprising one compound I, II or IV (component 1) and one further active substance (component 2), e.g. one active substance from groups A) to I), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e.g. two active substances from groups A) to I), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

The components can be used individually or already partially or completely mixed with one another to prepare the composition according to the invention. It is also possible for them to be packaged and used further as combination composition such as a kit of parts.

In one embodiment of the invention, the kits may include one or more, including all, components that may be used to prepare a subject agrochemical composition. E. g., kits may include one or more fungicide component(s) and/or an adjuvant component and/or an insecticide component and/or a growth regulator component and/or a herbicde. One or more of the components may already be combined together or pre-formulated. In those embodiments where more than two components are provided in a kit, the components may already be combined together and as such are packaged in a single container such as a vial, bottle, can, pouch, bag or canister. In other embodiments, two or more components of a kit may be packaged separately, i.e., not pre-formulated. As such, kits may include one or more separate containers such as vials, cans, bottles, pouches, bags or canisters, each container containing a separate component for an agrochemical composition. In both forms, a component of the kit may be applied separately from or together with the further components or as a component of a combination composition according to the invention for preparing the composition according to the invention.

The user applies the composition according to the invention usually from a predosage device, a knapsack sprayer, a spray tank or a spray plane. Here, the agrochemical composition is made up with water and/or buffer to the desired application concentration, it being possible, if appropriate, to add further auxiliaries, and the ready-to-use spray liquor or the agrochemical composition according to the invention is thus obtained. Usually, 50 to 500 liters of the ready-to-use spray liquor are applied per hectare of agricultural useful area, preferably 100 to 400 liters.

According to one embodiment, individual components of the composition according to the invention such as parts of a kit or parts of a binary or ternary mixture may be mixed by the user himself in a spray tank and further auxiliaries may be added, if appropriate (tank mix).

In a further embodiment, either individual components of the composition according to the invention or partially premixed components, e.g. components comprising compounds I, II and/or IV and/or active substances from the groups A) to I), may be mixed by the user in a spray tank and further auxiliaries and additives may be added, if appropriate (tank mix).

In a further embodiment, either individual components of the composition according to the invention or partially premixed components, e.g. components comprising compounds I, II and/or IV and/or active substances from the groups A) to I), can be applied jointly (e.g. after tankmix) or consecutively.

Preference is also given to mixtures comprising a compound I, II and/or IV (component 1) and at least one active substance selected from the strobilurines of group A) (component 2) and particularly selected from azoxystrobin, dimoxystrobin, fluoxastrobin, kresoxim-methyl, orysastrobin, picoxystrobin, pyraclostrobin and trifloxystrobin.

Preference is also given to mixtures comprising a compound I, II and/or IV (component 1) and at least one active substance selected from the carboxamides of group B) (component 2) and particularly selected from bixafen, boscalid, sedaxane, fenhexamid, metalaxyl, isopyrazam, mefenoxam, ofurace, dimethomorph, flumorph, fluopicolid (picobenzamid), zoxamide, carpropamid, mandipropamid and N-(3′,4′,5′-trifluorobiphenyl-2-yl)-3-difluoromethyl-1-methyl-1H-pyrazole-4-carboxamide.

Preference is given to mixtures comprising a compound of formula I, II and/or IV (component 1) and at least one active substance selected from the azoles of group C) (component 2) and particularly selected from cyproconazole, difenoconazole, epoxiconazole, fluquinconazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to mixtures comprising a compound I, II and/or IV (component 1) and at least one active substance selected from the heterocyclic compounds of group D) (component 2) and particularly selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, pro-quinazid, acibenzolar-S-methyl, captafol, folpet, fenoxanil, quinoxyfen and 5-ethyl-6-octyl-[1,2,4]triazolo[1,5-a]pyrimidine-7-ylamine.

Preference is also given to mixtures comprising a compound I, II and/or IV (component 1) and at least one active substance selected from the carbamates of group E) (component 2) and particularly selected from mancozeb, metiram, propineb, thiram, iprovalicarb, benthiavalicarb and propamocarb.

Preference is also given to mixtures comprising a compound I, II and/or IV (component 1) and at least one active substance selected from the fungicides given in group F) (component 2) and particularly selected from dithianon, fentin salts, such as fentin acetate, fosetyl, fosetyl-aluminium, H₃PO₃ and salts thereof, chlorthalonil, dichlofluanid, thiophanat-methyl, copper acetate, copper hydroxide, copper oxychloride, copper sulfate, sulfur, cymoxanil, metrafenone and spiroxamine.

Accordingly, the present invention furthermore relates to compositions comprising one compound I, II and/or IV (component 1) and one further active substance (component 2), which further active substance is selected from the column “Component 2” of the lines B-1 to B-346 of Table B.

A further embodiment relates to the compositions B-1 to B-346 listed in Table B, where a row of Table B corresponds in each case to a fungicidal composition comprising one of the in the present specification individualized compounds of formula I or II (component 1) and the respective further active substance from groups A) to I) (component 2) stated in the row in question. Preferably, the compositions described comprise the active substances in synergistically effective amounts.

TABLE B
Composition comprising one indiviualized compound I or II and one further
active substance from groups A) to I)

| Mixture | Component 1 | Component 2 |
|---|---|---|
| B-1 | one individualized compound I or II | Azoxystrobin |
| B-2 | one individualized compound I or II | Dimoxystrobin |
| B-3 | one individualized compound I or II | Enestroburin |
| B-4 | one individualized compound I or II | Fluoxastrobin |
| B-5 | one individualized compound I or II | Kresoxim-methyl |
| B-6 | one individualized compound I or II | Metominostrobin |
| B-7 | one individualized compound I or II | Orysastrobin |
| B-8 | one individualized compound I or II | Picoxystrobin |
| B-9 | one individualized compound I or II | Pyraclostrobin |
| B-10 | one individualized compound I or II | Pyribencarb |
| B-11 | one individualized compound I or II | Trifloxystrobin |
| B-12 | one individualized compound I or II | 2-(2-(6-(3-Chloro-2-methyl- |
| | | phenoxy)-5-fluoro-pyrimidin-4- |
| | | yloxy)-phenyl)-2-methoxyimino-N- |
| | | methyl-acetamide |
| B-13 | one individualized compound I or II | 2-(ortho-((2,5-Dimethylphenyl- |
| | | oxymethylen)phenyl)-3-methoxy- |
| | | acrylsauremethylester |
| B-14 | one individualized compound I or II | 3-Methoxy-2-(2-(N-(4-methoxy- |
| | | phenyl)-cyclopropanecarbox- |
| | | imidoylsulfanylmethyl)-phenyl)- |
| | | acrylic acid methyl ester |
| B-15 | one individualized compound I or II | 2-(2-(3-(2,6-dichlorophenyl)-1- |
| | | methyl-allylideneaminooxy- |
| | | methyl)-phenyl)-2-methoxyimino- |
| | | N-methyl-acetamide |
| B-16 | one individualized compound I or II | Benalaxyl |
| B-17 | one individualized compound I or II | Benalaxyl-M |
| B-18 | one individualized compound I or II | Benodanil |
| B-19 | one individualized compound I or II | Bixafen |
| B-20 | one individualized compound I or II | Boscalid |
| B-21 | one individualized compound I or II | Carboxin |
| B-22 | one individualized compound I or II | Fenfuram |
| B-23 | one individualized compound I or II | Fenhexamid |
| B-24 | one individualized compound I or II | Flutolanil |
| B-25 | one individualized compound I or II | Furametpyr |
| B-26 | one individualized compound I or II | Isopyrazam |
| B-27 | one individualized compound I or II | Isotianil |
| B-28 | one individualized compound I or II | Kiralaxyl |
| B-29 | one individualized compound I or II | Mepronil |
| B-30 | one individualized compound I or II | Metalaxyl |
| B-31 | one individualized compound I or II | Metalaxyl-M |
| B-32 | one individualized compound I or II | Ofurace |
| B-33 | one individualized compound I or II | Oxadixyl |
| B-34 | one individualized compound I or II | Oxycarboxin |
| B-35 | one individualized compound I or II | Penthiopyrad |
| B-36 | one individualized compound I or II | Sedaxane |
| B-37 | one individualized compound I or II | Tecloftalam |
| B-38 | one individualized compound I or II | Thifluzamide |
| B-39 | one individualized compound I or II | Tiadinil |
| B-40 | one individualized compound I or II | 2-Amino-4-methyl-thiazole-5- |
| | | carboxylic acid anilide |
| B-41 | one individualized compound I or II | 2-Chloro-N-(1,1,3-trimethyl-indan- |
| | | 4-yl)-nicotinamide |
| B-42 | one individualized compound I or II | N-(3′,4′,5′-trifluorobiphenyl-2-yl)-3- |
| | | difluoromethyl-1-methyl-1H- |
| | | pyrazole-4-carboxamide |
| B-43 | one individualized compound I or II | N-(4′-trifluoromethylthiobiphenyl- |
| | | 2-yl)-3-difluoromethyl-1-methyl- |
| | | 1H-pyrazole-4-carboxamide |
| B-44 | one individualized compound I or II | N-(2-(1,3-dimethyl-butyl)-phenyl)- |
| | | 1,3-dimethyl-5-fluoro-1H- |
| | | pyrazole-4-carboxamide |
| B-45 | one individualized compound I or II | N-(2-(1,3,3-trimethyl-butyl)- |
| | | phenyl)-1,3-dimethyl-5-fluoro-1H- |
| | | pyrazole-4-carboxamide |
| B-46 | one individualized compound I or II | Dimethomorph |
| B-47 | one individualized compound I or II | Flumorph |
| B-48 | one individualized compound I or II | Pyrimorph |
| B-49 | one individualized compound I or II | Flumetover |
| B-50 | one individualized compound I or II | Fluopicolide |
| B-51 | one individualized compound I or II | Fluopyram |
| B-52 | one individualized compound I or II | Zoxamide |
| B-53 | one individualized compound I or II | N-(3-Ethyl-3,5,5-trimethyl- |
| | | cyclohexyl)-3-formylamino-2- |
| | | hydroxy-benzamide |
| B-54 | one individualized compound I or II | Carpropamid |
| B-55 | one individualized compound I or II | Diclocymet |
| B-56 | one individualized compound I or II | Mandipropamid |
| B-57 | one individualized compound I or II | Oxytetracyclin |
| B-58 | one individualized compound I or II | Silthiofam |
| B-59 | one individualized compound I or II | N-(6-methoxy-pyridin-3-yl) cyclo- |
| | | propanecarboxylic acid amide |
| B-60 | one individualized compound I or II | Azaconazole |
| B-61 | one individualized compound I or II | Bitertanol |
| B-62 | one individualized compound I or II | Bromuconazole |
| B-63 | one individualized compound I or II | Cyproconazole |
| B-64 | one individualized compound I or II | Difenoconazole |
| B-65 | one individualized compound I or II | Diniconazole |
| B-66 | one individualized compound I or II | Diniconazole-M |
| B-67 | one individualized compound I or II | Epoxiconazole |
| B-68 | one individualized compound I or II | Fenbuconazole |
| B-69 | one individualized compound I or II | Fluquinconazole |
| B-70 | one individualized compound I or II | Flusilazole |
| B-71 | one individualized compound I or II | Flutriafol |
| B-72 | one individualized compound I or II | Hexaconazol |
| B-73 | one individualized compound I or II | Imibenconazole |
| B-74 | one individualized compound I or II | Ipconazole |
| B-75 | one individualized compound I or II | Metconazole |
| B-76 | one individualized compound I or II | Myclobutanil |
| B-77 | one individualized compound I or II | Oxpoconazol |
| B-78 | one individualized compound I or II | Paclobutrazol |
| B-79 | one individualized compound I or II | Penconazole |
| B-80 | one individualized compound I or II | Propiconazole |
| B-81 | one individualized compound I or II | Prothioconazole |
| B-82 | one individualized compound I or II | Simeconazole |
| B-83 | one individualized compound I or II | Tebuconazole |
| B-84 | one individualized compound I or II | Tetraconazole |
| B-85 | one individualized compound I or II | Triadimefon |
| B-86 | one individualized compound I or II | Triadimenol |
| B-87 | one individualized compound I or II | Triticonazole |
| B-88 | one individualized compound I or II | Uniconazole |
| B-89 | one individualized compound I or II | 1-(4-Chloro-phenyl)-2- |
| | | ([1,2,4]triazol-1-yl)-cycloheptanol |
| B-90 | one individualized compound I or II | Cyazofamid |
| B-91 | one individualized compound I or II | Imazalil |
| B-92 | one individualized compound I or II | Imazalil-sulfate |
| B-93 | one individualized compound I or II | Pefurazoate |
| B-94 | one individualized compound I or II | Prochloraz |
| B-95 | one individualized compound I or II | Triflumizole |
| B-96 | one individualized compound I or II | Benomyl |
| B-97 | one individualized compound I or II | Carbendazim |
| B-98 | one individualized compound I or II | Fuberidazole |
| B-99 | one individualized compound I or II | Thiabendazole |
| B-100 | one individualized compound I or II | Ethaboxam |
| B-101 | one individualized compound I or II | Etridiazole |
| B-102 | one individualized compound I or II | Hymexazole |
| B-103 | one individualized compound I or II | 2-(4-Chloro-phenyl)-N-[4-(3,4- |
| | | dimethoxy-phenyl)-isoxazol-5-yl]- |
| | | 2-prop-2-ynyloxy-acetamide |
| B-104 | one individualized compound I or II | Fluazinam |
| B-105 | one individualized compound I or II | Pyrifenox |
| B-106 | one individualized compound I or II | 3-[5-(4-Chloro-phenyl)-2,3- |
| | | dimethyl-isoxazolidin-3-yl]- |
| | | pyridine |
| B-107 | one individualized compound I or II | 3-[5-(4-Methyl-phenyl)-2,3-dimethyl- |
| | | isoxazolidin-3-yl]-pyridine |
| B-108 | one individualized compound I or II | 2,3,5,6-Tetrachloro-4- |
| | | methanesulfonyl-pyridine |
| B-109 | one individualized compound I or II | 3,4,5-Trichloro-pyridine-2,6- |
| | | dicarbonitrile |
| B-110 | one individualized compound I or II | N-(1-(5-Bromo-3-chloro-pyridin-2- |
| | | yl)-ethyl)-2,4-dichloro- |
| | | nicotinamide |
| B-111 | one individualized compound I or II | N-((5-Bromo-3-chloro-pyridin-2- |
| | | yl)-methyl)-2,4-dichloro- |
| | | nicotinamide |
| B-112 | one individualized compound I or II | Bupirimate |
| B-113 | one individualized compound I or II | Cyprodinil |
| B-114 | one individualized compound I or II | Diflumetorim |
| B-115 | one individualized compound I or II | Fenarimol |
| B-116 | one individualized compound I or II | Ferimzone |
| B-117 | one individualized compound I or II | Mepanipyrim |
| B-118 | one individualized compound I or II | Nitrapyrin |
| B-119 | one individualized compound I or II | Nuarimol |
| B-120 | one individualized compound I or II | Pyrimethanil |
| B-121 | one individualized compound I or II | Triforine |
| B-122 | one individualized compound I or II | Fenpiclonil |
| B-123 | one individualized compound I or II | Fludioxonil |
| B-124 | one individualized compound I or II | Aldimorph |
| B-125 | one individualized compound I or II | Dodemorph |
| B-126 | one individualized compound I or II | Dodemorph-acetate |
| B-127 | one individualized compound I or II | Fenpropimorph |
| B-128 | one individualized compound I or II | Tridemorph |
| B-129 | one individualized compound I or II | Fenpropidin |
| B-130 | one individualized compound I or II | Fluoroimid |
| B-131 | one individualized compound I or II | Iprodione |
| B-132 | one individualized compound I or II | Procymidone |
| B-133 | one individualized compound I or II | Vinclozolin |
| B-134 | one individualized compound I or II | Famoxadone |
| B-135 | one individualized compound I or II | Fenamidone |
| B-136 | one individualized compound I or II | Flutianil |
| B-137 | one individualized compound I or II | Octhilinone |
| B-138 | one individualized compound I or II | Probenazole |
| B-139 | one individualized compound I or II | 5-Amino-2-iso-propyl-4-ortho- |
| | | tolyl-2,3-dihydro-pyrazole-1- |
| | | carbothioic acid S-allyl ester |
| B-140 | one individualized compound I or II | Acibenzolar-S-methyl |
| B-141 | one individualized compound I or II | Amisulbrom |
| B-142 | one individualized compound I or II | Anilazin |
| B-143 | one individualized compound I or II | Blasticidin-S |
| B-144 | one individualized compound I or II | Captafol |
| B-145 | one individualized compound I or II | Captan |
| B-146 | one individualized compound I or II | Chinomethionat |
| B-147 | one individualized compound I or II | Dazomet |
| B-148 | one individualized compound I or II | Debacarb |
| B-149 | one individualized compound I or II | Diclomezine |
| B-150 | one individualized compound I or II | Difenzoquat, |
| B-151 | one individualized compound I or II | Difenzoquat-methylsulfate |
| B-152 | one individualized compound I or II | Fenoxanil |
| B-153 | one individualized compound I or II | Folpet |
| B-154 | one individualized compound I or II | Oxolinsaure |
| B-155 | one individualized compound I or II | Piperalin |
| B-156 | one individualized compound I or II | Proquinazid |
| B-157 | one individualized compound I or II | Pyroquilon |
| B-158 | one individualized compound I or II | Quinoxyfen |
| B-159 | one individualized compound I or II | Triazoxid |
| B-160 | one individualized compound I or II | Tricyclazole |
| B-161 | one individualized compound I or II | 2-Butoxy-6-iodo-3-propyl- |
| | | chromen-4-one |
| B-162 | one individualized compound I or II | 5-Chloro-1-(4,6-dimethoxy- |
| | | pyrimidin-2-yl)-2-methyl-1H- |
| | | benzoimidazole |
| B-163 | one individualized compound I or II | 5-Chloro-7-(4-methyl-piperidin-1- |
| | | yl)-6-(2,4,6-trifluoro-phenyl)- |
| | | [1,2,4]triazolo[1,5-a]pyrimidine |
| B-164 | one individualized compound I or II | 5-ethyl-6-octyl-[1,2,4]triazolo[1,5- |
| | | a]pyrimidine-7-ylamine |
| B-165 | one individualized compound I or II | Ferbam |
| B-166 | one individualized compound I or II | Mancozeb |
| B-167 | one individualized compound I or II | Maneb |
| B-168 | one individualized compound I or II | Metam |
| B-169 | one individualized compound I or II | Methasulphocarb |
| B-170 | one individualized compound I or II | Metiram |
| B-171 | one individualized compound I or II | Propineb |
| B-172 | one individualized compound I or II | Thiram |
| B-173 | one individualized compound I or II | Zineb |
| B-174 | one individualized compound I or II | Ziram |
| B-175 | one individualized compound I or II | Diethofencarb |
| B-176 | one individualized compound I or II | Benthiavalicarb |
| B-177 | one individualized compound I or II | Iprovalicarb |
| B-178 | one individualized compound I or II | Propamocarb |
| B-179 | one individualized compound I or II | Propamocarb hydrochlorid |
| B-180 | one individualized compound I or II | Valiphenal |
| B-181 | one individualized compound I or II | N-(1-(1-(4-cyanophenyl)ethane- |
| | | sulfonyl)-but-2-yl) carbamic acid- |
| | | (4-fluorophenyl) ester |
| B-182 | one individualized compound I or II | Dodine |
| B-183 | one individualized compound I or II | Dodine free base |
| B-184 | one individualized compound I or II | Guazatine |
| B-185 | one individualized compound I or II | Guazatine-acetate |
| B-186 | one individualized compound I or II | Iminoctadine |
| B-187 | one individualized compound I or II | Iminoctadine-triacetate |
| B-188 | one individualized compound I or II | Iminoctadine-tris(albesilate) |
| B-189 | one individualized compound I or II | Kasugamycin |
| B-190 | one individualized compound I or II | Kasugamycin-hydrochloride- |
| | | hydrate |
| B-191 | one individualized compound I or II | Polyoxine |
| B-192 | one individualized compound I or II | Streptomycin |
| B-193 | one individualized compound I or II | Validamycin A |
| B-194 | one individualized compound I or II | Binapacryl |
| B-195 | one individualized compound I or II | Dicloran |
| B-196 | one individualized compound I or II | Dinobuton |
| B-197 | one individualized compound I or II | Dinocap |
| B-198 | one individualized compound I or II | Nitrothal-isopropyl |
| B-199 | one individualized compound I or II | Tecnazen |
| B-200 | one individualized compound I or II | Fentin salts |
| B-201 | one individualized compound I or II | Dithianon |
| B-202 | one individualized compound I or II | Isoprothiolane |
| B-203 | one individualized compound I or II | Edifenphos |
| B-204 | one individualized compound I or II | Fosetyl, Fosetyl-aluminium |
| B-205 | one individualized compound I or II | Iprobenfos |
| B-206 | one individualized compound I or II | Phosphorous acid (H₃PO₃) and |
| | | derivatives |
| B-207 | one individualized compound I or II | Pyrazophos |
| B-208 | one individualized compound I or II | Tolclofos-methyl |
| B-209 | one individualized compound I or II | Chlorothalonil |
| B-210 | one individualized compound I or II | Dichlofluanid |
| B-211 | one individualized compound I or II | Dichlorophen |
| B-212 | one individualized compound I or II | Flusulfamide |
| B-213 | one individualized compound I or II | Hexachlorbenzene |
| B-214 | one individualized compound I or II | Pencycuron |
| B-215 | one individualized compound I or II | Pentachlorophenol and salts |
| B-216 | one individualized compound I or II | Phthalide |
| B-217 | one individualized compound I or II | Quintozene |
| B-218 | one individualized compound I or II | Thiophanate Methyl |
| B-219 | one individualized compound I or II | Tolylfluanid |
| B-220 | one individualized compound I or II | N-(4-chloro-2-nitro-phenyl)-N- |
| | | ethyl-4-methyl- |
| | | benzenesulfonamide |
| B-221 | one individualized compound I or II | Bordeaux mixture |
| B-222 | one individualized compound I or II | Copper acetate |
| B-223 | one individualized compound I or II | Copper hydroxide |
| B-224 | one individualized compound I or II | Copper oxychloride |
| B-225 | one individualized compound I or II | basic Copper sulfate |
| B-226 | one individualized compound I or II | Sulfur |
| B-227 | one individualized compound I or II | Biphenyl |
| B-228 | one individualized compound I or II | Bronopol |
| B-229 | one individualized compound I or II | Cyflufenamid |
| B-230 | one individualized compound I or II | Cymoxanil |
| B-231 | one individualized compound I or II | Diphenylamin |
| B-232 | one individualized compound I or II | Metrafenone |
| B-233 | one individualized compound I or II | Mildiomycin |
| B-234 | one individualized compound I or II | Oxin-copper |
| B-235 | one individualized compound I or II | Prohexadione calcium |
| B-236 | one individualized compound I or II | Spiroxamine |
| B-237 | one individualized compound I or II | Tolylfluanid |
| B-238 | one individualized compound I or II | N-(Cyclopropylmethoxyimino-(6- |
| | | difluoromethoxy-2,3-difluoro- |
| | | phenyl)-methyl)-2-phenyl |
| | | acetamide |
| B-239 | one individualized compound I or II | N′-(4-(4-chloro-3-trifluoromethyl- |
| | | phenoxy)-2,5-dimethyl-phenyl)-N- |
| | | ethyl-N-methyl formamidine |
| B-240 | one individualized compound I or II | N′-(4-(4-fluoro-3-trifluoromethyl- |
| | | phenoxy)-2,5-dimethyl-phenyl)-N- |
| | | ethyl-N-methyl formamidine |
| B-241 | one individualized compound I or II | N′-(2-methyl-5-trifluoromethyl-4- |
| | | (3-trimethylsilanyl-propoxy)- |
| | | phenyl)-N-ethyl-N-methyl formamidine |
| B-242 | one individualized compound I or II | N′-(5-difluoromethyl-2-methyl-4- |
| | | (3-trimethylsilanyl-propoxy)- |
| | | phenyl)-N-ethyl-N-methyl formamidine |
| B-243 | one individualized compound I or II | 2-{1-[2-(5-Methyl-3- |
| | | trifluoromethyl-pyrazole-1-yl)- |
| | | acetyl]-piperidin-4-yl}-thiazole-4- |
| | | carboxylic acid methyl-(1,2,3,4- |
| | | tetrahydro-naphthalen-1-yl)-amide |
| B-244 | one individualized compound I or II | 2-{1-[2-(5-Methyl-3-trifluoro- |
| | | methyl-pyrazole-1-yl)-acetyl]-piperidin- |
| | | 4-yl}-thiazole-4-carboxylic |
| | | acid methyl-(R)-1,2,3,4- |
| | | tetrahydro-naphthalen-1-yl-amide |
| B-245 | one individualized compound I or II | Acetic acid 6-tert-butyl-8-fluoro- |
| | | 2,3-dimethyl-quinolin-4-yl ester |
| B-246 | one individualized compound I or II | Methoxy-acetic acid 6-tert-butyl-8- |
| | | fluoro-2,3-dimethyl-quinolin-4-yl |
| | | ester |
| B-247 | one individualized compound I or II | Carbaryl |
| B-248 | one individualized compound I or II | Carbofuran |
| B-249 | one individualized compound I or II | Carbosulfan |
| B-250 | one individualized compound I or II | Methomylthiodicarb |
| B-251 | one individualized compound I or II | Bifenthrin |
| B-252 | one individualized compound I or II | Cyfluthrin |
| B-253 | one individualized compound I or II | Cypermethrin |
| B-254 | one individualized compound I or II | alpha-Cypermethrin |
| B-255 | one individualized compound I or II | zeta-Cypermethrin |
| B-256 | one individualized compound I or II | Deltamethrin |
| B-257 | one individualized compound I or II | Esfenvalerate |
| B-258 | one individualized compound I or II | Lambda-cyhalothrin |
| B-259 | one individualized compound I or II | Permethrin |
| B-260 | one individualized compound I or II | Tefluthrin |
| B-261 | one individualized compound I or II | Diflubenzuron |
| B-262 | one individualized compound I or II | Flufenoxuron |
| B-263 | one individualized compound I or II | Lufenuron |
| B-264 | one individualized compound I or II | Teflubenzuron |
| B-265 | one individualized compound I or II | Spirotetramate |
| B-266 | one individualized compound I or II | Clothianidin |
| B-267 | one individualized compound I or II | Dinotefuran |
| B-268 | one individualized compound I or II | Imidacloprid |
| B-269 | one individualized compound I or II | Thiamethoxam |
| B-270 | one individualized compound I or II | Acetamiprid |
| B-271 | one individualized compound I or II | Thiacloprid |
| B-272 | one individualized compound I or II | Endosulfan |
| B-273 | one individualized compound I or II | Fipronil |
| B-274 | one individualized compound I or II | Abamectin |
| B-275 | one individualized compound I or II | Emamectin |
| B-276 | one individualized compound I or II | Spinosad |
| B-277 | one individualized compound I or II | Spinetoram |
| B-278 | one individualized compound I or II | Hydramethylnon |
| B-279 | one individualized compound I or II | Chlorfenapyr |
| B-280 | one individualized compound I or II | Fenbutatin oxide |
| B-281 | one individualized compound I or II | Indoxacarb |
| B-282 | one individualized compound I or II | Metaflumizone |
| B-283 | one individualized compound I or II | Flonicamid |
| B-284 | one individualized compound I or II | Lubendiamide |
| B-285 | one individualized compound I or II | Chlorantraniliprole |
| B-286 | one individualized compound I or II | Cyazypyr (HGW86) |
| B-287 | one individualized compound I or II | Cyflumetofen |
| B-288 | one individualized compound I or II | Acetochlor |
| B-289 | one individualized compound I or II | Dimethenamid |
| B-290 | one individualized compound I or II | metolachlor |
| B-291 | one individualized compound I or II | Metazachlor |
| B-292 | one individualized compound I or II | Glyphosate |
| B-293 | one individualized compound I or II | Glufosinate |
| B-294 | one individualized compound I or II | Sulfosate |
| B-295 | one individualized compound I or II | Clodinafop |
| B-296 | one individualized compound I or II | Fenoxaprop |
| B-297 | one individualized compound I or II | Fluazifop |
| B-298 | one individualized compound I or II | Haloxyfop |
| B-299 | one individualized compound I or II | Paraquat |
| B-300 | one individualized compound I or II | Phenmedipham |
| B-301 | one individualized compound I or II | Clethodim |
| B-302 | one individualized compound I or II | Cycloxydim |
| B-303 | one individualized compound I or II | Profoxydim |
| B-304 | one individualized compound I or II | Sethoxydim |
| B-305 | one individualized compound I or II | Tepraloxydim |
| B-306 | one individualized compound I or II | Pendimethalin |
| B-307 | one individualized compound I or II | Prodiamine |
| B-308 | one individualized compound I or II | Trifluralin |
| B-309 | one individualized compound I or II | Acifluorfen |
| B-310 | one individualized compound I or II | Bromoxynil |
| B-311 | one individualized compound I or II | Imazamethabenz |
| B-312 | one individualized compound I or II | Imazamox |
| B-313 | one individualized compound I or II | Imazapic |
| B-314 | one individualized compound I or II | Imazapyr |
| B-315 | one individualized compound I or II | Imazaquin |
| B-316 | one individualized compound I or II | Imazethapyr |
| B-317 | one individualized compound I or II | 2,4-Dichlorophenoxyacetic acid |
| | | (2,4-D) |
| B-318 | one individualized compound I or II | Chloridazon |
| B-319 | one individualized compound I or II | Clopyralid |
| B-320 | one individualized compound I or II | Fluroxypyr |
| B-321 | one individualized compound I or II | Picloram |
| B-322 | one individualized compound I or II | Picolinafen |
| B-323 | one individualized compound I or II | Bensulfuron |
| B-324 | one individualized compound I or II | Chlorimuron-ethyl |
| B-325 | one individualized compound I or II | Cyclosulfamuron |
| B-326 | one individualized compound I or II | Iodosulfuron |
| B-327 | one individualized compound I or II | Mesosulfuron |
| B-328 | one individualized compound I or II | Metsulfuron-methyl |
| B-329 | one individualized compound I or II | Nicosulfuron |
| B-330 | one individualized compound I or II | Rimsulfuron |
| B-331 | one individualized compound I or II | Triflusulfuron |
| B-332 | one individualized compound I or II | Atrazine |
| B-333 | one individualized compound I or II | Hexazinone |
| B-334 | one individualized compound I or II | Diuron |
| B-335 | one individualized compound I or II | Florasulam |
| B-336 | one individualized compound I or II | Pyroxasulfone |
| B-337 | one individualized compound I or II | Bentazone |
| B-338 | one individualized compound I or II | Cinidon-ethlyl |
| B-339 | one individualized compound I or II | Cinmethylin |
| B-340 | one individualized compound I or II | Dicamba |
| B-341 | one individualized compound I or II | Diflufenzopyr |
| B-342 | one individualized compound I or II | Quinclorac |
| B-343 | one individualized compound I or II | Quinmerac |
| B-344 | one individualized compound I or II | Mesotrione |
| B-345 | one individualized compound I or II | Saflufenacil |
| B-346 | one individualized compound I or II | Topramezone |

The active substances referred to as component 2, their preparation and their activity against harmful fungi is known (cf.: http://www.alanwood.net/pesticides/); these sub-stances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The mixtures of active substances can be prepared as compositions comprising besides the active ingredients at least one inert ingredient by usual means, e.g. by the means given for the compositions of compounds I, II and/or IV.

Concerning usual ingredients of such compositions reference is made to the explanations given for the compositions containing compounds I, II and/or IV.

The mixtures of active substances according to the present invention are suitable as fungicides, as are the compounds of formula I, II ad IV. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, II and/or IV respectively.

The compounds I, II and IV and pharmaceutically acceptable salts thereof are also suitable for treating diseases in men and animals, especially as antimycotics, for treating cancer and for treating virus infections. The term “antimycotic”, as distinguished from the term “fungicide”, refers to a medicament for combating zoopathogenic or humanpathogenic fungi, i.e. for combating fungi in animals, especially in mammals (including humans) and birds.

Thus, a further aspect of the present invention relates to a medicament comprising at least one compound of the formulae I, II and/or IV and/or at least one pharmaceutically acceptable salt thereof and a pharmaceutically acceptable carrier.

Suitable pharmaceutically acceptable salts are especially physiologically tolerated salts of the compound I, in particular the acid addition salts with physiologically acceptable acids. Examples of suitable organic and inorganic acids are hydrochloric acid, hydrobromic acid, phosphoric acid, sulfuric acid, C₁-C₄-alkylsulfonic acids, such as methane-sulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Further suitable acids are described, for example, in Fortschritte der Arzneimittelforschung, Volume 10, pages 224 ff., Birkhauser Verlag, Basle and Stuttgart, 1966, the entire contents of which is expressly incorporated herein by way of reference.

Suitable carriers are, for example, solvents, carriers, excipients, binders and the like customarily used for pharmaceutical formulations, which are described below in an exemplary manner for individual types of administration.

A further aspect of the present invention relates to the use of compounds I, II and IV or of pharmaceutically acceptable salts thereof for preparing an antimycotic medicament; i.e. for preparing a medicament for the treatment and/or prophylaxis of infections with humanpathogenic and/or zoopathogenic fungi. Another aspect of the present invention relates to the use of compounds of formulae I, II and/or IV or of pharmaceutically acceptable salts thereof for preparing a medicament for the treatment of cancer. Another aspect of the present invention relates to the use of compounds of formulae I, II and/or IV or of pharmaceutically acceptable salts thereof for preparing a medicament for the treatment or prophylaxis of virus infections.

The compounds of formulae I, II and IV and/or their pharmaceutically acceptable salts are suitable for the treatment, inhibiton or control of growth and/or propagation of tumor cells and the disorders associated therewith. Accordingly, they are suitable for cancer therapy in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The compounds of formulae I, II and IV and/or their pharmaceutically acceptable salts are suitable for the therapy of cancer or cancerous disorders of the following organs: breast, lung, intestine, prostate, skin (melanoma), kidney, bladder, mouth, larynx, oesophagus, stomach, ovaries, pancreas, liver and brain or CNS.

The compounds of formulae I, II and IV and/or their pharmaceutically acceptable salts are suitable for the treatment of virus infections in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like. They are suitable for treating virus infections like retrovirus infections such as HIV and HTLV, influenza virus infection, rhinovirus infections, herpes and the like.

The compounds according to the invention can be administered in a customary manner, for example orally, intravenously, intramuscularly or subcutaneously. For oral administration, the active compound can be mixed, for example, with an inert diluent or with an edible carrier; it can be embedded into a hard or soft gelatin capsule, it can be compressed to tablets or it can be mixed directly with the food/feed. The active compound can be mixed with excipients and administered in the form of indigestible tablets, buccal tablets, pastilles, pills, capsules, suspensions, potions, syrups and the like. Such preparations should contain at least 0.1% of active compound. The composition of the preparation may, of course, vary. It usually comprises from 2 to 60% by weight of active compound, based on the total weight of the preparation in question (dosage unit). Preferred preparations of the compound I according to the invention comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like. Capsules may furthermore comprise a liquid carrier. Other substances which modify the properties of the dosage unit may also be used. For example, tablets, pills and capsules may be coated with schellack, sugar or mixtures thereof. In addition to the active compound, syrups or potions may also comprise sugar (or other sweeteners), methyl- or propylparaben as preservative, a colorant and/or a flavor. The components of the active compound preparations must, of course, be pharmaceutically pure and nontoxic at the quantities employed. Furthermore, the active compounds can be formulated as preparations with a controlled release of active compound, for example as delayed-release preparations.

The active compounds can also be administered parenterally or intraperitoneally. Solutions or suspensions of the active compounds or their salts can be prepared with water using suitable wetting agents, such as hydroxypropylcellulose. Dispersions can also be prepared using glycerol, liquid polyethylene glycols and mixtures thereof in oils. Frequently, these preparations furthermore comprise a preservative to prevent the growth of microorganisms.

Preparations intended for injections comprise sterile aqueous solutions and dispersions and also sterile powders for preparing sterile solutions and dispersions. The preparation has to be sufficiently liquid for injection. It has to be stable under the preparation and storage conditions and it has to be protected against contamination by microorganisms. The carrier may be a solvent or a dispersion medium, for example, water, ethanol, a polyol (for example glycerol, propylene glycol or liquid polyethylene glycol), a mixture thereof and/or a vegetable oil.

The invention is further illustrated by the following, non-limiting examples.

### I. SYNTHESIS EXAMPLES

### General Experimental Procedures

¹H and ¹³C NMR spectra were obtained on a Bruker AC 400 spectrometer at 400 MHz. Proton spectra were referenced to tetramethylsilane as an internal standard and the carbon spectra were referenced to CDCl₃ (purchased from Aldrich or Cambridge Isotope Laboratories, unless otherwise specified). Melting points were obtained on a Mel-Temp II apparatus and are uncorrected. ESI Mass spectra were obtained on a Agilent LCMSDVL Mass Spectrometer. HPLC analyses were obtained using an Eclipse XDB C18 Column utilizing PDA detection at 254 nm (unless otherwise specified) on a Agilent Prominence HPLC system. The following time program was utilized (flow rate of 1.0 mL per minute):

| | Percent A | Percent B |
|---|---|---|
| | (H₂O with | (CH₃CN with |
| Time | 0.1% TFA) | 0.1% TFA) |
| 0.00 | 95 | 5 |
| 10.00 | 5 | 95 |
| 12.00 | 0 | 100 |
| 15.00 | 95 | 5 |
| 20.00 | 95 | 5 |

### 1. Preparation of 1-(2-(2,4-dichlorophenethyl)-2-hydroxy-3,3-dimethylbutyl)-1H-1,2,4-triazole-5(4H)-thione (compound I.A.15)

### 1.1 1-(2,4-Dichlorophenyl)-4,4-dimethylpent-1-en-3-one

A solution of 2,4-dichlorobenzaldehyde (5.00 g, 28.5 mmol) and pinacolone (2.80 g, 28.5 mmol) in ethanol (25 mL) was added dropwise to a stirred and chilled (0° C.) solution of sodium hydroxide (4.50 g, 114 mmol) in water (25 mL). The mixture was stirred at this temperature for 1 h and then was warmed to room temperature, and stirring was continued for 15 h. The solvent was evaporated under reduced pressure. The residue was diluted with ethyl acetate (EtOAc) (100 mL) and washed with water (2×50 mL), brine (50 mL), dried over Na₂SO₄ and concentrated under reduced pressure. The crude product was purified by column chromatography (silica gel, eluent: 1% EtOAc in hexanes) to afford the titled ketone, yield: 6.0 g (80%).

¹H NMR (400 MHz, CDCl₃) δ 7.98 (d, J=15.6 Hz, 1H), 7.59 (d, J=8.4 Hz, 1H), 7.43 (s, 1H), 7.26 (d, J=8.0 Hz, 1H), 7.09 (d, J=15.6 Hz, 1H), 1.23 (s, 9H).

### 1.2 1-(2,4-Dichlorophenyl)-4,4-dimethylpentan-3-one

A solution of the ketone obtained in step 1.1 (6.0 g, 25.2 mmol) in EtOAc (30 mL) was added to a solution of Raney-nickel (3.0 g, excess) in EtOAc (30 mL) and the reaction mixture was stirred under hydrogen (1 atm) for 16 h. The reaction mixture was filtered through a Celite plug and concentrated under reduced pressure to afford 5.5 g of crude product. The crude material was used directly in the next reaction step without further purification.

¹H NMR (400 MHz, CDCl₃) δ 7.35 (s, 1H), 7.19-7.14 (m, 2H), 2.95 (t, J=7.4 Hz, 2H), 2.79 (t, J=7.4 Hz, 2H), 1.10 (s, 9H).

### 1.3 2-tert-Butyl-2-(2,4-dichlorophenethyl)-oxirane

A solution of dimethyl sulfide (4.22 g, 68.0 mmol) in acetonitrile (15 mL) was added to a stirred and cooled (0° C.) solution of dimethyl sulfate (7.67 g, 60.4 mmol) in acetonitrile (15 mL). The mixture was warmed to room temperature and stirred for 16 h. After this, the ketone obtained in step 1.2 (5.50 g, 20.1 mmol) in DMSO (15 mL) was added, followed by the addition of powdered KOH (5.60 g, 100 mmol, 5.0 equiv). The mixture was stirred at room temperature for an additional 16 h. Then the reaction mixture was diluted with water (50 mL) and extracted with EtOAc (3×100 mL). Upon separation, the combined organic layers were dried over Na₂SO₄ and concentrated under reduced pressure. The crude product was purified by column chromatography (silica gel, eluent: 1% EtOAc in hexanes) to afford the titled oxirane, yield: 4.10 g (70%).

¹H NMR (400 MHz, CDCl₃) δ 7.35 (s, 1H), 7.18-7.13 (m, 2H), 2.81 (d, J=4.0 Hz, 1H), 2.68 (d, J=4.0 Hz, 1H), 2.61-2.57 (m, 2H), 2.13-2.05 (m, 1H), 1.97-1.90 (m, 1H), 0.97 (s, 9H).

### 1.4 3-((1H-[1,2,4-]Triazol-1-yl)-methyl)-1-(2,4-dichlorophenyl)-4,4-dimethylpentan-3-ol

A solution of [1,2,4]-triazole (1.89 g, 27.4 mmol) in DMF (15 mL) was added to a stirred and chilled (0° C.) suspension of NaH (1.09 g, 27.4 mmol) in DMF (7 mL). After stirring at 0° C. for 1 h, the reaction mixture was warmed to room temperature and stirred for an additional 1 h. A solution of the oxirane obtained in step 1.3 (5.00 g, 18.3 mmol, 1.0 equiv) in DMF (8 mL) was added, and the reaction mixture was heated at 80° C. for 7 h. The mixture was then poured into cold water (60 mL) and extracted with EtOAc (3×100 mL). Upon separation, the combined organic layers were dried over Na₂SO₄ and concentrated under reduced pressure. The crude product was purified by column chromatography (silica gel, eluent: 40% EtOAc in hexanes) to afford the titled triazole; yield: 1.50 g (25%).

¹H NMR (400 MHz, CDCl₃) δ 8.23 (s, 1H), 8.02 (s, 1H), 7.30 (d, J=2.0 Hz, 1H), 7.13 (dd, J₁=2.0 Hz, J_{2=8.0} Hz, 1H), 6.98 (d, J=8.0 Hz, 1H), 4.40-4.32 (m, 2H), 3.23 (s, 1H), 2.67-2.60 (m, 1H), 1.95-1.59 (m, 3H), 1.04 (s, 9H).

### 1.5 1-(2-(2,4-Dichlorophenethyl)-2-hydroxy-3,3-dimethylbutyl)-1H-[1,2,4]-triazole-5(4H)-thione

Sulfur powder (469 mg, 14.6 mmol) was added to a solution of the triazole obtained in step 1.4 (500 mg, 1.46 mmol) in N-methylpyrrolidinone (10 mL) and heated to 180° C. for 24 h. The reaction mixture was cooled, quenched by brine (30 mL), and diluted with EtOAc (75 mL). The organic layer was separated and washed with brine (4×75 mL), then separated, dried over Na₂SO₄ and concentrated under reduced pressure. The crude product was purified by column chromatography (silica gel, eluent: 30% EtOAc in hexanes) to afford the titled thiotriazole as an off-white solid, yield: 300 mg (55%).

m.p. 137-138° C.;

¹H NMR (400 MHz, CDCl₃) δ 12.41 (bs, 1H), 7.89 (s, 1H), 7.28-7.12 (m, 3H), 4.48-4.47 (m, 2H), 3.74 (s, 1H), 2.82-2.75 (1H), 2.45-2.37 (m, 1H), 1.90-1.86 (m, 2H), 1.10 (s, 9H);

MS (ESI) m/z 374.3 [M]⁺.

The compounds of examples 2 to 4 (in case of example 4: except for the last step) were prepared analogously.

### 2. 1-(2-(2,4-Difluorophenethyl)-2-hydroxy-3,3-dimethylbutyl)-1H-1,2,4-triazole-5(4H)-thione (compound I.A.6)

Off-white solid; yield: 160 mg (48%);

m.p. 130-132° C.;

¹H NMR (400 MHz, CDCl₃) δ 12.10 (bs, 1H), 7.88 (s, 1H), 7.14-7.08 (m, 1H), 6.77-6.67 (m, 2H), 4.47 (s, 2H), 3.73 (s, 1H), 2.71-2.64 (s, 1H), 2.33-2.25 (m, 1H), 1.91-1.87 (m, 2H), 1.09 (s, 9H);

MS (ESI) m/z 342.1 [M+H]⁺.

### 3. 1-(2-(3,4-Dichlorophenethyl)-2-hydroxy-3,3-dimethylbutyl)-1H-1,2,4-triazole-5(4H)-thione (compound I.A.18)

Off-white solid; yield: 350 mg (64%);

m.p. 155-157° C.;

¹H NMR (400 MHz, CDCl₃) δ 12.45 (bs, 1H), 7.90 (s, 1H), 7.29-7.23 (m, 2H), 6.97 (d, J=8.0 Hz, 1H), 4.51-4.45 (m, 2H), 3.62 (s, 1H), 2.67-2.59 (m, 1H), 2.22-2.15 (m, 1H), 1.95-1.89 (m, 2H), 1.08 (s, 9H);

MS (ESI) m/z 374.0 [M]⁺.

### 4. 1-(2-(4-Fluorophenethyl)-2-hydroxy-3,3-dimethylbutyl)-1H-1,2,4-triazole-5(4H)-thione (compound I.A.4)

A solution of n-BuLi in hexane (1.6M, 3.22 mL, 5.15 mmol, 2.5 equiv) was added to a solution of 3-((1H-1,2,4-triazol-1-yl)-methyl)-1-(4-fluorophenyl)-4,4-dimethylpentan-3-ol (600 mg, 2.06 mmol, 1.0 equiv) in THF (15 mL) at −20° C. After stirring at −20° C. for 1 h, the reaction mixture was warmed to 0° C. and stirred for an additional 1 h. At this time, sulfur powder (198 mg, 3.09 mmol, 1.5 equiv) was added, and stirring continued for an additional 2 h at 0° C. The reaction mixture was diluted with EtOAc (30 mL) and washed with saturated NH₄Cl solution (25 mL) and brine (25 mL). The organic layer was separated, dried over Na₂SO₄ and concentrated under reduced pressure. The crude product was purified by silica gel column chromatography (eluent: 35% EtOAc/hexanes) to afford the titled thiotriazole as an off-white solid; yield: 170 mg (25%).

m.p. 134-135° C.;

¹H NMR (400 MHz, CDCl₃) δ 12.20 (br s, 1H), 7.87 (s, 1H), 7.11-7.08 (m, 2H), 6.94-6.90 (m, 2H), 4.48 (s, 2H), 3.63 (br s, 1H), 2.70-2.62 (m, 1H), 2.25-2.18 (m, 1H), 1.95-1.90 (m, 2H), 1.09 (s, 9H);

MS (ESI) m/z 324.1 [M+H]⁺.

### 5. Preparation of 1-(2-hydroxy-3,3-dimethyl-2-(4-methylphenethyl)butyl)-1H-1,2,4-triazole-5(4H)-thione (compound I.A.33)

### 5.1 4,4-Dimethyl-1-p-tolylpent-1-en-3-one

A solution of 4-methylbenzaldehyde (5.00 g, 41.6 mmol) and pinacolone (4.16 g, 41.6 mmol) in ethanol (30 mL) was added dropwise to a stirred and chilled (0° C.) solution of sodium hydroxide (6.65 g, 166 mmol) in water (30 mL). The mixture was stirred at this temperature for 1 h, warmed to room temperature, and stirring was continued for 15 h. The solvent was evaporated under reduced pressure. The residue was diluted with EtOAc (75 mL), and the organic layer was washed with water (2×50 mL), brine (50 mL), dried over Na₂SO₄ and concentrated under reduced pressure. The crude product was purified by column chromatography (silica gel, eluent: 1% EtOAc in hexanes) to afford the titled ketone, yield: 6.5 g (77%).

¹H NMR (400 MHz, CDCl₃) δ 7.67 (d, J=15.6, 1H), 7.48 (d, J=8.0, 2H), 7.20 (d, J=7.6, 2H), 7.10 (d, J=15.6, 1H), 2.38 (s, 3H), 1.23 (s, 9H).

### 5.2 2-tert-Butyl-2-(4-methylstyryl)-oxirane

A solution of dimethyl sulfide (6.76 g, 1.09 mmol) in acetonitrile (15 mL) was added to a stirred and chilled (0° C.) solution of dimethyl sulfate (12.3 g, 96.3 mmol) in acetonitrile (15 mL). The mixture was warmed to room temperature and stirred for 16 h. After this time, the ketone obtained in step 5.1 (6.50 g, 32.1 mmol) in DMSO (15 mL) was added, followed by the addition of powdered KOH (9.02 g, 160 mmol, 5.0 equiv). The mixture was stirred at room temperature for an additional 16 h. After this time, the reaction mixture was diluted with water (75 mL) and extracted with EtOAc (3×100 mL). Upon separation, the combined organic layers were dried over Na₂SO₄ and concentrated under reduced pressure to afford 6.2 g of crude product. The crude material was used directly in the next reaction step without further purification.

¹H NMR (400 MHz, CDCl₃) δ 7.29 (d, J=8.0 Hz, 2H), 7.19-7.12 (m, 2H), 6.64-6.52 (m, 2H), 2.98 (d, J=5.2, 1 H), 2.57 (d, J=5.6, 1 H), 2.34 (s, 3H), 1.03 (s, 9H).

### 5.3 3-((1H-1,2,4-Triazol-1-yl)methyl)-4,4-dimethyl-1-p-tolylpent-1-en-3-ol

A solution of [1,2,4]-triazole (1.67 g, 24.3 mmol) in DMF (10 mL) was added to a stirred and chilled (0° C.) suspension of NaH (972 mg, 24.3 mmol) in DMF (5 mL). After stirring at 0° C. for 1 h, the reaction mixture was warmed to room temperature and stirred for an additional 1 h. A solution of the oxirane obtained in step 5.2 (3.50 g, 16.2 mmol, 1.0 equiv) in DMF (5 mL) was added, and the reaction mixture was heated at 80° C. for 7 h. Then the mixture was poured into cold water (60 mL) and extracted with EtOAc (3×75 mL). Upon separation, the combined organic layers were dried over Na₂SO₄ and concentrated under reduced pressure. The crude product was purified by column chromatography (silica gel, eluent: 40% EtOAc in hexanes) to afford the titled triazole; yield: 2.80 g (60%).

¹H NMR (400 MHz, CDCl₃) δ 8.06 (s, 1H), 7.85 (s, 1H), 7.11-7.05 (m, 4H), 6.37 (d, J=16.0 Hz, 1H), 6.14 (d, J=16.0 Hz, 1H), 4.48-4.37 (m, 2H), 3.57 (s, 1H), 2.31 (s, 3H), 1.09 (s, 9H).

### 5.4. 3-((1H-1,2,4-Triazol-1-yl)methyl)-4,4-dimethyl-1-p-tolylpentan-3-ol

To a solution of the triazole obtained in step 5.3 (2.80 g, 9.82 mmol) in EtOH (20 mL) was added 10% Pd/C (500 mg) and the reaction mixture was stirred under hydrogen (40 psi) for 1 h. The reaction mixture was filtered through a celite plug and concentrated under reduced pressure. The crude product was washed with hexane (5×50 mL) to afford the titled compound, yield: 1.80 g (64%).

¹H NMR (400 MHz, CDCl₃) δ 8.21 (s, 1H), 8.01 (s, 1H), 7.05 (d, J=7.6, 2H), 6.93 (d, J=7.6, 2H), 4.36 (s, 2H), 3.08 (s, 1H), 2.46-2.40 (m, 1H), 1.92 (s, 3H), 1.91-1.69 (m, 3H), 1.03 (s, 9H).

### 5.5 1-(2-Hydroxy-3,3-dimethyl-2-(4-methylphenethyl)butyl)-1H-1,2,4-triazole-5(4H)-thione

Sulfur powder (557 mg, 17.4 mmol) was added to a solution of the triazole obtained in step 5.4 (500 mg, 1.74 mmol) in N-methylpyrrolidinone (10 mL) and heated to 180° C. for 24 h. The reaction mixture was cooled and quenched by the addition of brine (30 mL) and diluted with EtOAc (100 mL). The organic layer was washed with brine (4×75 mL), separated, dried over Na₂SO₄ and concentrated under reduced pressure. The crude product was purified by column chromatography (silica gel, eluent: 35% EtOAc in hexanes) to afford the titled thiotriazole as an off-white solid; yield: 350 mg (60%).

m.p. 137-138° C.;

¹H NMR (400 MHz, CDCl₃) δ 12.6 (bs, 1H), 7.85 (s, 1H), 7.05-7.03 (m, 4H), 4.53-4.43 (m, 2H), 3.66 (bs, 1H), 2.68-2.61 (m, 1H), 2.29 (s, 3H), 2.21-2.13 (m, 1H), 1.96-1.92 (m, 2H), 1.08 (s, 9H);

MS (ESI) m/z 320.1 [M+H]⁺.

The compound of example 6 was prepared analogously.

### 6. 1-(2-Hydroxy-3,3-dimethyl-2-(4-(trifluoromethyl)phenethyl)butyl)-1H-1,2,4-triazole-5(4H)-thione (compound I.A.41)

Off-white solid; yield: 700 mg (60%);

m.p. 139-140° C.;

¹H NMR (400 MHz, CDCl₃) δ 11.96 (bs, 1H), 7.87 (s, 1H), 7.49 (d, J=8.0 Hz, 2H), 7.27 (m, 2H), 4.53-4.44 (m, 2H), 3.68 (bs, 1H), 2.78-2.71 (m, 1H), 2.35-2.27 (m, 1H), 1.98-1.93 (m, 2H), 1.09 (s, 9H);

MS (ESI) m/z 374.0 [M+H]⁺.

### II. EXAMPLES OF THE ACTION AGAINST HARMFUL FUNGI

The fungicidal action of the compounds of the formulae I and II was demonstrated by the following experiments:

### A) Greenhouse Tests

The spray solutions were prepared in several steps:

The stock solution were prepared by adding a mixture of acetone and/or dimethylsulfoxide and the wetting agent/emulsifier Wettol, which is based on ethoxylated alkylphenoles, in a relation (volume) solvent-emulsifier of 99 to 1 to 25 mg of the compound to give a total of 10 ml. Water was then added to total volume of 100 ml. This stock solution was diluted with the described solvent-emulsifier-water mixture to the given concentration.

1. Control of Late Blight on Tomatoes Caused by Phytophthora infestans

Young seedlings of tomato plants were grown in pots. These plants were sprayed to run-off with an aqueous suspension, containing the concentration of active ingredient or their mixture mentioned in the table below. The next day, the treated plants were inoculated with an aqueous suspension of sporangia of Phytophthora infestans. After inoculation, the trial plants were immediately transferred to a humid chamber. After 6 days at 18 to 20° C. and a relative humidity close to 100% the extent of fungal attack on the leaves was visually assessed as % diseased leaf area. The plants which had been treated with an aqueous active compound preparation comprising 300 ppm of the active compound of example 1 showed an infection of 15%, whereas the untreated plants were 90% infected.

2. Preventative Control of Grey Mold (Botrytis cinerea) on Leaves of Green Pepper

Young seedlings of green pepper were grown in pots to the 2 to 3 leaf stage. These plants were sprayed to run-off with an aqueous suspension, containing the concentration of active ingredient or their mixture mentioned in the table below. The next day the treated plants were inoculated with a spore suspension of Botrytis cinerea in a 2% aqueous biomalt solution. Then the trial plants were immediately transferred to a dark, humid chamber. After 5 days at 22 to 24° C. and a relative humidity close to 100% the extent of fungal attack on the leaves was visually assessed as % diseased leaf area.

The plants which had been treated with an aqueous active compound preparation comprising 300 ppm of the active compound of example 4 showed an infection of 7%, whereas the untreated plants were 90% infected.

3. Preventative Control of Brown Rust on Wheat Caused by Puccinia recondita

The first two developed leaves of pot-grown wheat seedling were sprayed to run-off with an aqueous suspension, containing the concentration of active ingredient or their mixture as described below. The next day the plants were inoculated with spores of Puccinia recondita. To ensure the success the artificial inoculation, the plants were transferred to a humid chamber without light and a relative humidity of 95 to 99% and 20 to 24° C. for 24 h. Then the trial plants were cultivated for 6 days in a greenhouse chamber at 20-24° C. and a relative humidity between 65 and 70%. The extent of fungal attack on the leaves was visually assessed as % diseased leaf area. The plants which had been treated with an aqueous active compound preparation comprising 300 ppm of the active compounds of examples 3 and 6 showed an infection of at most 15%, whereas the untreated plants were 60% infected.

4. Preventative Control of Leaf Blotch on Wheat Caused by Septoria tritici

Leaves of pot-grown wheat seedling were sprayed to run-off with an aqueous suspension of the active compound or their mixture, prepared as described. The plants were allowed to air-dry. On the following day the plants were inoculated with an aqueous spore suspension of Septoria tritici. Then the trial plants were immediately transferred to a humid chamber at 18-22° C. and a relative humidity close to 100%. After 4 days the plants were transferred to a chamber with 18-22° C. and a relative humidity close to 70%. After 4 weeks the extent of fungal attack on the leaves was visually assessed as % diseased leaf area. The plants which had been treated with an aqueous active compound preparation comprising 300 ppm of the active compounds of examples 3 and 6 showed an infection of at most 10%, whereas the untreated plants were 90% infected.

1-22. (canceled)

23. A compound of formula I or II 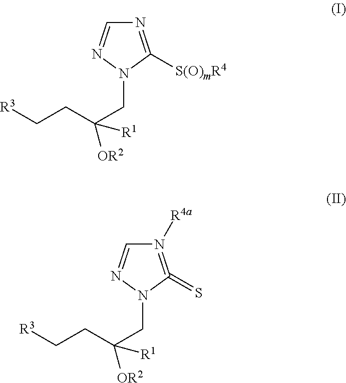
wherein
R¹ is selected from the group consisting of C₁-C₁₀-alkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, C₃-C₁₀-halocycloalkyl-C₁-C₄-alkyl, wherein the cycloalkyl moiety in the 4 last-mentioned radicals may carry 1, 2, 3 or 4 substituents R⁶, phenyl which may carry 1, 2, 3, 4 or 5 substituents R⁵, and a saturated, partially unsaturated or fully unsaturated 3-, 4-, 5-, 6- or 7-membered heterocyclic ring containing 1, 2 or 3 heteroatoms or heteroatom-containing groups selected from the group consisting of N, O, S, SO and SO₂ as ring members, wherein the heterocyclic ring may carry 1, 2 or 3 substituents R⁵;
R² is selected from the group consisting of hydrogen and a protective group;
R³ is selected from the group consisting of C₃-C₁₀-cycloalkyl, C₃-C₁₀-halocycloalkyl, wherein the cycloalkyl moiety in the 2 last-mentioned radicals may carry 1, 2, 3 or 4 substituents R⁶, C₃-C₁₀-cycloalkenyl, C₃-C₁₀-halocycloalkenyl, wherein the cycloalkenyl moiety in the 2 last-mentioned radicals may carry 1, 2, 3 or 4 substituents R⁶, aryl which may carry 1, 2 or 3 substituents R⁷, and a saturated, partially unsaturated or fully unsaturated 3-, 4-, 5-, 6- or 7-membered heterocyclic ring containing 1, 2 or 3 heteroatoms or heteroatom-containing groups selected from the group consisting of N, O, S, SO and SO₂ as ring members, wherein the heterocyclic ring may carry 1, 2 or 3 substituents R⁸;
R⁴ is selected from the group consisting of hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R⁹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R⁹; or, in case m is 0, may also be selected from the group consisting of —C(═O)R¹⁰, —C(═S)R¹⁰, —S(O)₂R¹⁰, —CN, —P(=Q)R¹¹R¹², M and a group of the formula III 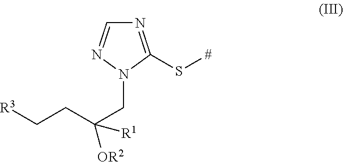
wherein
R¹, R² and R³ are as defined for formulae I and II; and
# is the attachment point to the remainder of the molecule;
R^{4a} is selected from the group consisting of hydrogen, C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R⁹, and a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R⁹, —C(═O)R¹⁰, —C(═S)R¹⁰, —S(O)₂R¹⁰, —CN, —P(=Q)R¹¹R¹² and M;
each R⁵ is independently selected from the group consisting of halogen, nitro, CN, C₁-C₄-alkyl, C₁-C₄-haloalkyl, C₁-C₄-alkoxy, C₁-C₄-haloalkoxy and NR¹³R¹⁴;
each R⁶ is independently selected from the group consisting of nitro, CN, C₁-C₄-alkyl, C₁-C₄-haloalkyl, C₁-C₄-alkoxy, C₁-C₄-haloalkoxy and NR¹³R¹⁴;
each R⁷ is independently selected from the group consisting of halogen, nitro, CN, C₁-C₄-alkyl, C₁-C₄-haloalkyl, C₂-C₄-alkenyl, C₂-C₄-haloalkenyl, C₁-C₄-alkox_{Y}, C₁-C₄-haloalkoxy, phenyl, phenoxy, wherein the phenyl moiety in the two last-mentioned phenyl radicals may carry 1, 2 or 3 substituents R⁵, and NR¹³R¹⁴;
each R⁸ is independently selected from the group consisting of halogen, nitro, CN, C₁-C₄-alkyl, C₁-C₄-haloalkyl, —C₄-alkenyl, C₂-C₄-haloalkenyl, C₁-C₄-alkoxy, C₁-C₄-haloalkoxy, phenyl, phenoxy, wherein the phenyl moiety in the two last-mentioned phenyl radicals may carry 1, 2 or 3 substituents R⁵, and NR¹³R¹⁴;
each R⁹ is independently selected from the group consisting of halogen, nitro, CN, C₁-C₄-alkyl, C₁-C₄-haloalkyl, C₁-C₄-alkoxy, C₁-C₄-haloalkoxy and NR¹³R¹⁴;
R¹⁰ is selected from the group consisting of hydrogen, C₁-C₁₀-alkyl, C₁-C₁₀-haloalkyl, C₁-C₁₀-alkoxy, C₁-C₁₀-haloalkoxy, C₁-C₁₀-aminoalkyl, C₃-C₁₀-cycloalkyl, C₃-C₁₀-halocycloalkyl, phenyl, phenyl-C₁-C₄-alkyl, wherein the phenyl moiety in the 2 last-mentioned radicals may carry 1, 2, 3, 4 or 5 substituents R⁹, a 5- or 6-membered saturated, partially unsaturated or aromatic heterocyclic ring containing 1, 2 or 3 heteroatoms selected from the group consisting of N, O and S as ring members, wherein the heterocyclic ring may carry 1, 2 or 3 substituents R⁹, and NR¹³R¹⁴;
R¹¹ and R¹², independently of each other, are selected from the group consisting of C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₀-cycloalkyl, C₃-C₁₀-halocycloalkyl, alkoxy, C₁-C₁₀-haloalkoxy, C₁-C₄-alkoxy-C₁-C₁₀-alkyl, C₁-C₄-alkoxy-C₁-C₁₀-alkoxy, C₁-C₁₀-alkylthio, C₁-C₁₀-haloalkylthio, C₂-C₁₀-alkenyloxy, C₂-C₁₀-alkenylthio, C₂-C₁₀-alkynyloxy, C₂-C₁₀, alkynylthio, C₃-C₁₀-cycloalkoxy, C₃-C₁₀-cycloalkylthio, phenyl, phenyl-C₁-C₄-alkyl, phenylthio, phenyl-C₁-C₄-alkoxy, and NR¹³R¹⁴;
each R¹³ is independently selected from the group consisting of hydrogen and C₁-C₈-alkyl;
each R¹⁴ is independently selected from the group consisting of hydrogen, C₁-C₈-alkyl, phenyl, and phenyl-C₁-C₄-alkyl;
or R¹³ and R¹⁴ together form a linear C₄- or C₅-alkylene bridge or a group —CH₂CH₂OCH₂CH₂— or —CH₂CH₂NR¹⁵CH₂CH₂—;
each R¹⁵ is independently selected from the group consisting of hydrogen and C₁-C₄-alkyl;
Q is O or S;
M is a metal cation equivalent or an ammonium cation of formula (NR^{a}R^{b}R^{c}R^{d})⁺, wherein R^{a}, R^{b}, R^{c} and R^{d}, independently of each other, are selected from the group consisting of hydrogen, C₁-C₁₀-alkyl, phenyl and benzyl, wherein the phenyl moiety in the 2 last-mentioned radicals may carry 1, 2 or 3 substituents independently selected from the group consisting of halogen, CN, nitro, C₁-C₄-alkyl, C₁-C₄-haloalkyl, C₁-C₄-alkoxy, C₁-C₄-haloalkoxy and NR¹³R¹⁴; and
m is 0, 1, 2 or 3;
with the proviso that R³ is not 4-chlorophenyl, 2-pyridyl or 2-furyl if R¹ is tert-butyl or optionally substituted cyclopropyl;
and/or the agriculturally acceptable salt thereof.

24. The compound of claim 23, wherein R¹ is selected from the group consisting of C₁-C₆-alkyl, C₁-C₄-haloalkyl, C₃-C₆-cycloalkyl, C₃-C₆-halocycloalkyl, C₃-C₆-cycloalkyl-C₁-C₂-alkyl, C₃-C₆-halocycloalkyl-C₁-C₂-alkyl, wherein the cycloalkyl moiety in the 4 last-mentioned radicals may carry 1 or 2 substituents selected from the group consisting of methyl, difluoromethyl and trifluoromethyl, phenyl which may carry 1, 2, 3, 4 or 5 substituents R⁵, and a 5- or 6-membered heteroaromatic ring containing 1, 2 or 3 heteroatoms selected from the group consisting of N, O and S as ring members, wherein the heteroaromatic ring may carry 1, 2 or 3 substituents R⁵.

25. The compound of claim 24, wherein R¹ is selected from the group consisting of tert-butyl, phenyl, cyclopropyl, 1-methylcyclopropyl, 1-chlorocyclopropyl and 1-cyclopropylethyl.

26. The compound of claim 23, wherein the protective group in the definition of R² is selected from the group consisting of C₁-C₆-alkyl, C₁-C₄-haloalkyl, C₂-C₆-alkenyl, C₂-C₄-haloalkenyl, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylaminocarbonyl, and di-(C₁-C₄-alkyl)-aminocarbonyl.

27. The compound of claim 23, wherein R² is hydrogen.

28. The compound of claim 23, wherein R³ is selected from the group consisting of phenyl, which may carry 1, 2 or 3 substituents R⁷, and a 5- or 6-membered heteroaromatic ring containing 1, 2 or 3 heteroatoms selected from the group consisting of N, O and S as ring members, wherein the heteroaromatic ring may carry 1, 2 or 3 substituents R⁸.

29. The compound of claim 28, wherein R³ is phenyl which may carry 1, 2 or 3 substituents R⁷.

30. The compound of claim 28, wherein R⁷ is selected from the group consisting of C₁-C₄-alkyl, C₁-C₂-haloalkyl, C₂-C₄-alkenyl, C₁-C₄-alkoxy and C₁-C₂-haloalkoxy.

31. The compound of claim 23, wherein R³ is selected from the group consisting of 2-chlorophenyl, 3-chlorophenyl, 2,4-dichlorophenyl, 3,4-dichlorophenyl and phenyl carrying 1, 2 or 3 fluorine substituents.

32. The compound of claim 23, wherein R³ is selected from the group consisting of 2-chloro-4-fluorophenyl, 2-fluoro-4-chlorophenyl, 3-chloro-4-fluorophenyl and 3-fluoro-4-chlorophenyl.

33. The compound of claim 23, wherein R¹⁰ is selected from the group consisting of C₁-C₄-alkyl, C₁-C₂-haloalkyl, C₁-C₄-alkoxy, C₁-C₂-haloalkoxy, phenyl, phenoxy and NR¹³R¹⁴, wherein R¹³ is hydrogen and R¹⁴ is selected from the group consisting of hydrogen, C₁-C₄-alkyl and phenyl or the two of R¹³ and R¹⁴ are C₁-C₄-alkyl.

34. The compound of claim 23, wherein R⁴ is selected from the group consisting of hydrogen, C₁-C₄-alkyl, —C(═O)R¹⁰, —S(O)₂R¹⁰, —CN, M and a group of the formula III.

35. The compound of claim 33, wherein R⁴ is selected from the group consisting of hydrogen, CN, —C(═O)CH₃, —C(═O)OCH₃ and methyl.

36. The compound of claim 23, wherein R^{4a} is selected from the group consisting of hydrogen, C₁-C₄-alkyl and —C(═O)R¹⁰.

37. The compound of claim 23, wherein m is 0.

38. An agricultural composition comprising at least one compound of claim 23, or an agriculturally acceptable salt thereof and a liquid or solid carrier.

39. A method for controlling harmful fungi, comprising treating the fungi, their habitat or the materials or plants to be protected against fungal attack, or the soil or propagation material with an effective amount of at least compound of claim 23.

40. A seed treated with at least compound of claim 23, in an amount of from 0.1 g to 10 kg per 100 kg of seeds.

41. A pharmaceutical composition comprising at least one compound of claim 23 or a pharmaceutically acceptable salt thereof and at least one pharmaceutically acceptable carrier.

42. A method for treating cancer or virus infections or for combating zoopathogenic or humanpathogenic fungi, which comprises treating an individual in need thereof with at least one compound of claim 23, or a pharmaceutically acceptable salt thereof.
